(19) United States
(12) Patent Application Publication
(10) Pub. No.: US 2024/0180847 A1
(43) Pub. Date: Jun. 6, 2024

(54) EXTRACELLULAR VESICLES LOADED WITH AT LEAST TWO DIFFERENT NUCLEIC ACIDS

(71) Applicant: Carmine Therapeutics Pte. Ltd., Singapore (SG)

(72) Inventors: Yumi Kawamura, Singapore (SG); Ronne Wee Yeh Yeo, Singapore (SG)

(73) Assignee: Carmine Therapeutics Pte. Ltd., Singapore (SG)

(21) Appl. No.: 18/284,989

(22) PCT Filed: Mar. 31, 2022
(86) PCT No.: PCT/SG2022/050187

§ 371 Date: Sep. 29, 2023

Related U.S. Application Data
(60) Provisional application No. 63/169,161, filed on Mar. 31, 2021.

(51) Int. Cl. A61K 9/51; A61K 31/713; A61P 37/06; C12N 9/22; C12N 15/113; C12N 15/88

(52) U.S. Cl. CPC A61K 9/5176; A61K 31/713; A61P 37/06; C12N 9/22; C12N 15/113; C12N 15/88

(57) ABSTRACT

Extracellular vesicles loaded with at least two different nucleic acids. An extracellular vesicle loaded with a cargo, wherein the cargo comprises at least two different nucleic acids and methods for preparing and using such extracellular vesicles.

### CROSS-REFERENCE TO RELATED APPLICATIONS

The present application claims the benefit of U.S. Provisional Application No. 63/169, 161 filed Mar. 31, 2021, the content of which is hereby incorporated herein by reference in its entirety.

### BACKGROUND

Nucleic acid therapeutics represent novel modalities that enable therapeutic intervention at the genetic level. These include, but are not limited to, short RNAs such as antisense oligonucleotides (ASOs), short interfering RNAS (siRNAs) and microRNAs (miRNAs), long RNAs such as messenger RNAs (mRNA), or even double-stranded DNA (dsDNA). These modalities are usually deemed disease modifying as they either inhibit or promote the production of the disease-related protein. With the discovery of genome editing tools such as CRISPR-Cas9, zinc finger nucleases (ZFNs), and transcription activator-like effector nucleases (TALENs), diseases can now be fundamentally corrected at the genome level. However, the clinical development of nucleic acids as next-generation drugs has been impeded mainly due to limitations in delivery, a consequence of their inability to penetrate cell membranes, immunogenicity and vulnerability to nucleases in the circulation.

Presently, most nucleic acid therapeutics involve a single target, as exemplified by approved drugs such as Patisiran (siRNA against hereditary transthyretin-mediated amyloidosis), Voretigene neparvovec (AAV gene therapy against Leber's congenital amaurosis), or the Pfizer-BioNTech COVID-19 mRNA vaccine. However, there are several therapeutic applications that will benefit from the delivery of two or more gene products. One such example is the concept of vectorized antibodies, or antibody gene therapy, where antibodies are produced endogenously from a patient's organ such as the liver, following a gene therapy treatment involving the delivery of two transgenes encoding the heavy and light chains of the antibody. Another example is genome editing which requires an efficient vehicle for the co-delivery of its multiple components. Specifically, gene insertion mediated by the CRISPR-Cas9 system requires a transcript for Cas9 and guide RNA, as well as DNA template for homology-directed repair. Prime editing involves the co-delivery of a transcript encoding the SpCas9 endonuclease fused to an engineered reverse transcriptase, as well as a prime editing guide RNA (pegRNA).

Two of the most advanced and widely explored delivery vehicles are namely adeno-associated viruses (AAVs) and lipid nanoparticles (LNPs). AAVs are small (20 nm), replication-defective viruses that are capable of infecting both dividing and quiescent cells. These viruses are actively employed for gene therapy applications as they are not known to cause disease and elicit a comparatively milder immune response as compared to other viruses at the same dose. However, they are limited by their small transgene capacity (4.5 kb) and cannot be redosed due to adaptive immune responses upon redosing. LNPs are the next class of delivery vehicles and their ability to deliver RNA to hepatocytes and dendritic cells has been clinically validated. However, LNPs are relatively unstable in circulation and systemic delivery of nucleic acids to tissues other than hepatocytes remains highly challenging.

Extracellular vesicles (EVs) are cell-derived lipid membrane-bound vesicles that mediate the transfer of biomolecules among cells. EVs are biocompatible, have a unique native tropism, and depending on their cellular origin, they pose little threats of toxicity or immunogenicity. We have previously demonstrated that red blood cell EVs (RBCEVs) can be loaded exogenously with a wide variety of payloads including ASOs, mRNA and DNA (for example, see PCT/SG2021/050020, the contents of which are incorporated entirely herein).

The present invention has been devised in light of the above considerations.

### SUMMARY

Provided herein is an extracellular vesicle loaded with a cargo, wherein the cargo comprises at least two different nucleic acid molecules. In some cases, the cargo comprises at least three different nucleic acid molecules.

The nucleic acid molecule may be selected from the group consisting of: a DNA plasmid, an RNA plasmid, a circular DNA, a linear double-stranded DNA, a DNA minicircle, a dumbbell-shaped DNA minimal vector, an RNA minicircle, a small interfering RNA (siRNA), a messenger RNA (mRNA), a guide RNA (gRNA), a prime editing guide RNA (peg RNA), a CRISPR RNA (crRNA), a trans-activating CRISPR RNA (tracrRNA), a circular RNA, a microRNA (miRNA), a primary miRNA (pri-miRNA), a precursor miRNA (pre-miRNA), a piwi-interacting RNA (piRNA), a transfer RNA (tRNA), a long noncoding RNA (IncRNA), an antisense oligonucleotide (ASO), a short hairpin RNA (shRNA), a small activating RNA (saRNA), a small nucleolar RNAs (snoRNA), a gapmer, a locked nucleic acid (LNA), a peptide nucleic acid (PNA), and an expression vector.

The at least two different nucleic acid molecules or at least three different nucleic acid molecules may have non-identical sequences. Additionally or alternatively, the at least two different nucleic acid molecules or at least three different nucleic acids may be different types of nucleic acid molecule. For example, the cargo may comprise at least two different molecules, wherein each of the at least two different molecules is independently selected from the group consisting of a small interfering RNA (SiRNA), a messenger RNA (mRNA), a guide RNA (gRNA), a prime editing guide RNA (peg RNA), a CRISPR RNA (crRNA), a trans-activating CRISPR RNA (tracrRNA), a circular RNA, a microRNA (miRNA), a primary miRNA (pri-miRNA), a precursor miRNA (pre-miRNA), a piwi-interacting RNA (piRNA), a transfer RNA (tRNA), a long noncoding RNA (IncRNA), an antisense oligonucleotide (ASO), a short hairpin RNA (shRNA), a small activating RNA (saRNA), a small nucleolar RNAs (snoRNA), a gapmer, a locked nucleic acid (LNA), a peptide nucleic acid (PNA), an expression vector, a DNA plasmid, a circular DNA, a linear double-stranded DNA, an RNA plasmid, a DNA minicircle, dumbbell-shaped DNA minimal vector, and an RNA minicircle. Preferably, the at least two different nucleic acid molecules or at least three different nucleic acid molecules comprise an mRNA or a DNA plasmid encoding a Cas enzyme and a gRNA. The gRNA is preferably a pegRNA or a sgRNA. In some cases, the cargo may comprise a DNA and an RNA, at least two non-identical molecules, at least two different mRNA, a plasmid and a gRNA, or an mRNA and a gRNA. In some cases, the at least two different nucleic acid molecules comprise a plasmid, such as a DNA plasmid, and an antisense oligonucleotide. In some cases, the at least two different nucleic acid molecules comprise a plasmid, such as a DNA plasmid, and an SiRNA.

In certain aspects, the extracellular vesicle comprises a component of the CRISPR/Cas gene editing system. In some aspects, the extracellular vesicle comprises at least two or at least three components of the CRISPR/Cas gene editing system. For example, the extracellular vesicle may comprise a gRNA such as a sgRNA and a nucleic acid molecule encoding a nuclease. The nuclease may be a Cas9 or a Cas12 nuclease. The extracellular vesicle may comprise a DNA repair template. As such, the extracellular vesicle may comprise one, two or three components selected from the group consisting of a gRNA, a nucleic acid encoding a nuclease, and a DNA repair template.

In another aspect, the extracellular vesicle comprises nucleic acid encoding an antigen-binding molecule or a fragment or an antigen-binding molecule. The antigen-binding molecule or fragment thereof may be an antibody, scFv, Fab, F(ab)₂, minibody or diabody. The extracellular vesicle may comprise a first nucleic acid encoding a first polypeptide of the antigen-binding molecule or fragment thereof, and a second nucleic acid encoding a further polypeptide of the antigen-binding molecule or fragment thereof.

In preferred aspects, the extracellular vesicle is an extracellular vesicle derived from a red blood cell.

Also described herein are compositions comprising extracellular vesicles as disclosed herein. In these compositions, at least one of the extracellular vesicles comprises a cargo, wherein the cargo comprises at least two different nucleic acids. In some cases, the composition comprises a plurality of extracellular vesicles. In such compositions, one, several, or substantially all of the extracellular vesicles in the composition may comprise cargo, wherein the cargo comprises at least two different nucleic acid molecules or at least three different nucleic acid molecules.

Also described herein are methods for preparing extracellular vesicles comprising a cargo, wherein the cargo comprises at least two different nucleic acid molecules. In some cases, the method comprises a) providing a mixture, the mixture comprising nucleic acid molecules to be loaded into an extracellular vesicle; and b) contacting the mixture with an extracellular vesicle under conditions sufficient for the extracellular vesicle to be loaded with the nucleic acid molecules, wherein the mixture of nucleic acid molecules comprises at least two different nucleic acid molecules. In some aspects, the mixture further comprises a transfection reagent. In others, the method further comprises a step of electroporating, after the mixture is contacted with the extracellular vesicle. In some methods, the mixture comprises the at least two different cargo molecules in a ratio of about 1:1. Preferably, the extracellular vesicle to be loaded is a red blood cell derived extracellular vesicle (RBCEV).

Some methods described herein involve a step of preparing the mixture comprising nucleic acid molecules to be loaded into an extracellular vesicle. This step may involve preparing a mixture of nucleic acid molecules, wherein at least one of the nucleic acid molecules is different to other nucleic acid molecules in the mixture, wherein transfection reagent is added to this mixture. Alternatively, this step may involve the preparation of two or more sub-mixtures, each sub-mixture comprising a nucleic acid to be loaded into an extracellular vesicle and a transfection reagent. The sub-mixtures are then combined to form the mixture.

The at least two different cargo molecules is each a nucleic acid molecule. The at least two different cargo molecules may both be plasmids. The at least two different cargo molecules may both be RNA molecules. The at least two different nucleic acid molecules may have non-identical sequences and/or the at least two different nucleic acid molecules may be different types of nucleic acid molecule (e.g., at least one DNA and at least one RNA, at least one plasmid and at least one oligonucleotide, at least one plasmid and at least one RNA, at least one circular and at least one non-circular nucleic acid, etc., as will be clear to those skilled in the art reading the present disclosure).

The nucleic acids of the mixture may be selected from the group consisting of: a small interfering RNA (SIRNA), a messenger RNA (mRNA), a guide RNA (gRNA), a prime editing guide RNA (peg RNA), a CRISPR RNA (crRNA), a trans-activating CRISPR RNA (tracrRNA), a circular RNA, a microRNA (miRNA), a primary miRNA (pri-miRNA), a precursor miRNA (pre-miRNA), a piwi-interacting RNA (piRNA), a transfer RNA (tRNA), a long noncoding RNA (IncRNA), an antisense oligonucleotide (ASO), a short hairpin RNA (shRNA), a small activating RNA (saRNA), a small nucleolar RNAs (snoRNA), a gapmer, a locked nucleic acid (LNA), a peptide nucleic acid (PNA), an expression vector, a DNA plasmid, an RNA plasmid, a DNA minicircle, dumbbell-shaped DNA minimal vector and an RNA minicircle. The cargo may comprise a DNA and an RNA, at least two non-identical molecules, at least two different mRNA, a plasmid and a gRNA, or an mRNA and a gRNA.

As is readily appreciated upon consideration of the disclosure provided herein, co-encapsulated nucleic acids may be of approximately the same size, or may be of different sizes. Indeed, in some embodiments, co-encapsulated nucleic acids may share gross common structural features (e.g., being a plasmid, including expression control elements, which may in some embodiments be the same, and including different expressed [aka “payload”] sequences), or may even be substantially identical but for specific sequence and/or other structural variations. Alternatively, as is taught herein (and confirmed by exemplification), co-loaded nucleic acids may be of very different sizes (e.g., one or more plasmids and one or more oligonucleotides).

Certain methods involve the loading of nucleic acid molecules, wherein the nucleic acid molecules comprise or encode components of a CRISPR/Cas gene editing system. In such methods, the mixture may comprise or encode components of a CRISPR/Cas gene editing system. The mixture may comprise a gRNA molecule and a nucleic acid molecule encoding a nuclease. The nuclease may be a Cas9 nuclease or a Cas12 nuclease.

Certain methods involve the loading of cargo molecules, wherein the cargo molecules comprise two or more nucleic acid molecules that encode an antigen binding molecule. In such methods, the mixture may comprise a first nucleic acid encoding a first polypeptide of the antigen-binding molecule, and a second nucleic acid encoding a second polypeptide of the antigen-binding molecule.

In another aspect, described herein is a method for delivering two or more nucleic acid molecules to a cell, wherein the method comprises contacting the cell with an extracellular vesicle according to the invention.

Also described herein is an extracellular vesicle for use in a method of treatment, a method of treatment, and the use of an extracellular vesicle in the manufacture of a medicament for the treatment of a disease or disorder. These aspects may involve the administration of an extracellular vesicle according to the invention to a subject with a genetic disorder, inflammatory disease, cancer, autoimmune disease, cardiovascular disease or a gastrointestinal disease. The subject may have cancer, the cancer optionally selected from leukemia, lymphoma, myeloma, breast cancer, lung cancer, liver cancer, colorectal cancer, nasopharyngeal cancer, kidney cancer or glioma. These aspects may involve the treatment of a disease in the patient by expression of a protein or peptide encoded by the nucleic acid. They may involve the treatment of disease by gene editing or gene therapy.

The invention includes the combination of the aspects and preferred features described except where such a combination is clearly impermissible or expressly avoided.

### BRIEF DESCRIPTION OF THE DRAWINGS

Embodiments and experiments illustrating the principles of the invention will now be discussed with reference to the accompanying figures in which:

FIG. 1. RBCEV loading of 2 plasmids. RBCEVs were loaded with plasmids encoding for copGFP (CMV-copGFP) or tdTomato (CMV-tdTomato) separately and co-transfected or loaded simultaneously (co-loaded) and transfected to 293T and HepG2 cells. 293T cells were transfected with 0.084 pmol DNA and HepG2 cells were transfected with 0.168 pmol DNA. Epi-fluorescent images of 293T cells (A) or HepG2 cells (B) taken 48 hours after transfection of RBCEVs and corresponding dot plots of cells analyzed by flow cytometry (PE vs. FITC). Colocalization analysis of 2-color channel images of 293T cells or HepG2 cells using the Pearson's correlation coefficient to measure the degree of correlation of colocalizing signals. Percentages of GFP and tdTomato positive 293T cells or HepG2 cells gated in the FITC channel and PE channel. Error bars represent mean±s.d., n=3, *p<0.05, **p<0.001.

FIG. 2. RBCEV loading of trastuzumab light chain (CAG-LC) and heavy chain (CAG-HC) plasmids. RBCEVs were co-loaded with LC and HC, loaded with LC and HC separately, or loaded with single bicistronic vector with IRES (CAG-LC-IRES-HC) or P2A (CAG-LC-P2A-HC) and transfected to 293T and HepG2 cells at equimolar DNA amount. 48 hours after transfection, the soluble trastuzumab levels in the cell culture supernatant were quantified by ELISA (A). DNA loading efficiency of plasmids calculated based on starting amount of plasmids added to the loading reaction and final amount of plasmids recovered after the loading reaction (B). Error bars represent mean±s.d., n=3, *p<0.05. Serum Trastuzumab expression from SCID mice injected with RBCEVs co-loaded with LC and HC (co-load), or RBCEVs loaded with LC and HC separately and mixed at a 1:1 ratio (co-transfect) at day 7, 14, 21, and 28 after injection (C). Error bars represent mean±s.d., n=5, *p<0.05.

FIG. 3. RBCEV loading of 3 plasmids. RBCEVs were loaded with 3 different plasmids each encoding for unique transgenes. Loaded RBCEVs were transfected to 293T cells at equimolar DNA amount. 48 hours after transfection, RNA was extracted and gene expression was measured by transcript levels determined by qRT-PCR. Expression was normalized to GAPDH. Error bars represent mean±s.d., n=3, *p<0.05, **p<0.001.

FIG. 4. RBCEV loading of DNA and mRNA. RBCEVs were loaded with different nucleic acid types. RBCEVs were loaded with tdTomato mRNA or DNA encoding for copGFP (CMV-copGFP) separately and co-transfected or loaded simultaneously (co-loaded) and transfected to 293T at equimolar DNA amount. Epi-fluorescent images of 293T cells (A) taken 48 hours after transfection of RBCEVs and corresponding dot plots of cells analyzed by flow cytometry (PE vs. FITC). Colocalization analysis of 2-color channel images of 293T cells (B) using the Pearson's correlation coefficient to measure the degree of correlation of colocalizing signals. Percentages of GFP and tdTomato positive 293T cells (C) gated in the FITC channel and PE channel. Error bars represent mean±s.d., n=3, *p<0.001.

FIG. 5. RBCEV loading of fluorescently-labelled DNA. DNA was labeled with a fluorophore (MFP488 or Cy5) using the Label IT Nucleic Acid Labeling Kit (Mirus Bio). RBCEVs were loaded with MFP488 or Cy5-labeled DNA, or simultaneously loaded with equal amounts of MFP488 and Cy5-labeled DNA. Loaded RBCEVs were analyzed by flow cytometry.

FIG. 6. RBCEVs co-loaded with double-stranded oligonucleotides can increase transgene expression in multiple cell lines. Huh-7 (A-C), HepG2 (D-F), and THP-1 cells (G-I) were transfected with RBCEVs loaded with DNA plasmid alone (EV-NP) or co-loaded with DNA plasmid and NF-κB decoy (ODN), scrambled (SCD), phosphorothioate-modified NF-κB decoy (ODN-PS), or phosphorothioate-modified scrambled (SCD-PS) oligonucleotides at increasing dosages from 12.5 to 100 pmol. 48 hours after transfection, cells were analyzed for gene expression (A, D, G), mean fluorescence intensity (B, E, H) and cell viability by flow cytometry (C, F, I).

FIG. 7. RBCEVs co-loaded with NF-κB decoy oligonucleotide can increase hFIX-HiBit expression in BL/6 mice. Luminescence of HiBiT-tagged FIX protein was measured from day 1 to day 49 after administration of RBCEVs loaded with DNA plasmid alone (0) or co-loaded with DNA plasmid and NF-κB decoy (ODN) or scrambled (SCD) oligonucleotides at 25 pmol (low dose; ODN-25 and SCD-25) or 100 pmol (high dose; ODN-100 and SCD-100).

FIG. 8. RBCEVs co-loaded with NF-κB decoy oligonucleotide can increase antibody expression in SCID mice. Trastuzumab was measured in the serum of mice from day 1 to day 49 after administration of RBCEVs loaded with DNA plasmid alone (0) or co-loaded with DNA plasmid and NF-κB decoy (ODN) oligonucleotides at 4 mg/kg and 6 mg/kg dose.

FIG. 9. RBCEVs co-loaded with DNA vector and NF-κB decoy oligonucleotides can reduce systemic IFNa and IFNb in vivo. BL/6 mice were injected with RBCEVs loaded with DNA plasmid alone (EV-NP) or co-loaded with DNA plasmid and NF-κB decoy (ODN) or scrambled (SCD) oligonucleotides at 100 pmol (ODN-100 and SCD-100) by intravenous tail-vein injection. Naked DNA plasmid was injected via hydrodynamic injection (HDI). Blood was drawn at different timepoints and quantified for mouse interferon alpha (IFNa; A) and interferon beta (IFNb; B) levels by ELISA.

FIG. 10. Comparison of oligonucleotide designs co-loaded in RBCEVs in vitro. HepG2 (A-C) and Huh7 (D-F) cells were transfected with RBCEVs co-loaded with a CMV-copGFP plasmid and an oligonucleotide of different design. GFP expression (A, D), mean fluorescence intensity (MFI; B, E) and cell viability using propidium iodide (PI) staining (C, F) were measured at 24 hour and 48 hour timepoints.

FIG. 11. Test of RBCEV loading efficiency with different bait oligonucleotide designs. RBCEVs were co-loaded with plasmid DNA and bait oligonucleotides from Table 2 or NF-κB decoy oligonucleotide. Plasmid and oligonucleotide loading efficiencies were measured with agarose gel electrophoresis of DNA extracted from loaded RBCEVs.

FIG. 12. RBCEVs co-loaded with a bait oligonucleotide can improve transgene expression in vitro. Huh7 cells were transfected with RBCEVs co-loaded with plasmid and bait oligonucleotide or NF-κB decoy oligonucleotide. FIX-Hibit protein expression (A) and EGFP expression (B) were measured at 24 hours after transfection.

FIG. 13. RBCEVs co-loaded with a double-stranded or a single-stranded oligonucleotide can increase transgene expression in vitro. Huh7 (A) and HepG2 (B) cells were transfected with RBCEVS co-loaded with a DNA plasmid that expresses hFIX-HiBit luciferase reporter construct and a double-stranded oligonucleotide (scrambled, NF-κB decoy) or a single-stranded oligonucleotide. Luminescence was measured 24 hours later with NANO-GLO® HiBIT assay.

FIG. 14. RBCEVs co-loaded with single-stranded oligonucleotides can modulate gene expression associate with immune response in vitro. THP-1 cells were transfected with RBCEVs co-loaded with a DNA plasmid that expresses hFIX-HiBit luciferase reporter construct and a double-stranded oligonucleotide (scrambled, NF-κB decoy) or a single-stranded oligonucleotide. Gene expression was measured 6 hours later by quantitative PCR (qPCR). Taqman target specific probes for IFNb1 (A), IL6 (B), CXCL10 (C) and CCL2 (D) were used for target genes. Taqman target specific probes for GAPDH was used for normalization of cDNA input.

### DETAILED DESCRIPTION OF CERTAIN EMBODIMENTS

Aspects and embodiments of the present invention will now be discussed with reference to the accompanying figures. Further aspects and embodiments will be apparent to those skilled in the art. All documents mentioned in this text are incorporated herein by reference.

### Extracellular Vesicles

The term “extracellular vesicle” (EV) as used herein refers to a small vesicle-like structure released from a cell into the extracellular environment. In particularly preferred aspects disclosed herein, the extracellular vesicles are derived from red blood cells (RBCEVs).

Extracellular vesicles (EVs) are substantially spherical fragments of plasma membrane or endosomal membrane between 50 and 1000 nm in diameter. Extracellular vesicles are released from various cell types under both pathological and physiological conditions. Extracellular vesicles have a membrane.

The membrane may be a double layer membrane (i.e. a lipid bilayer). The membrane may originate from the plasma membrane. Accordingly, the membrane of the extracellular vesicle may have a similar composition to the cell from which it is derived. In some aspects disclosed herein, the extracellular vesicles are substantially transparent.

The term extracellular vesicles encompasses exosomes, microvesicles, membrane microparticles, ectosomes, blebs and apoptotic bodies. Extracellular vesicles may be produced via outward budding and fission of cellular membrane. The production may be a natural process, or a chemically induced or enhanced process. In some aspects disclosed herein, the extracellular vesicle is a microvesicle produced via chemical induction.

Extracellular vesicles may be classified as exosomes, microvesicles or apoptotic bodies, based on their origin of formation. Microvesicles are a particularly preferred class of extracellular vesicle according to the invention disclosed herein. Preferably, the extracellular vesicles of the invention have been shed from the plasma membrane, and do not originate from the endosomal system. In certain aspects described herein, the extracellular vesicles are not exosomes. In some cases the extracellular vesicles are non-exosomal EVs.

In some aspects and embodiments of the present disclosure the extracellular vesicle is not an exosome. In some aspects and embodiments of the present disclosure the extracellular vesicle is not an ectosome. In some aspects and embodiments of the present disclosure the extracellular vesicle is not a bleb. In some aspects and embodiments of the present disclosure the extracellular vesicle is not an apoptotic body.

In some aspects and embodiments of the present disclosure the extracellular vesicle is a microvesicle or a membrane microparticle.

Extracellular vesicles disclosed herein may be derived from various cells, such as red blood cells, white blood cells, cancer cells, stem cells, dendritic cells, macrophages and the like. In a preferred embodiment, the extracellular vesicles are derived from a red blood cell, although extracellular vesicles from any source may be used, such as from cell lines. In preferred aspects described herein, the extracellular vesicles are derived from red blood cells.

Microvesicles or microparticles arise through direct outward budding and fission of the plasma membrane. Microvesicles are typically larger than exosomes, having diameters ranging from 100-500 nm. In some cases, a composition of microvesicles comprises microvesicles with diameters ranging from 50-1000 nm, from 50-750 nm, from 50-500 nm, from 50-300 nm, from 50-200 nm, from 50-150 nm, from 101-1000 nm, from 101-750 nm, from 101-500 nm, from 101-300 nm, from 100-300 nm, or from 100-200 nm. Preferably, the diameters are from 100-300 nm.

A population of microvesicles, for example as present in a composition, pharmaceutical composition, medicament or preparation, will comprise microvesicles with a range of different diameters, the median diameter of microvesicles within a microvesicle sample can range 50-1000 nm, from 50-750 nm, from 50-500 nm, from 50-300 nm, from 50-200 nm, from 50-150 nm, from 101-1000 nm, from 101-750 nm, from 101-500 nm, from 101-300 nm, from 100-300 nm, from 100-200 nm, or from 100-150 nm. Preferably, the median diameter is in one of the ranges: 50-300 nm, 50-200 nm, 50-150 nm, 100-300 nm, 100-200 nm, or 100-150 nm. The mean average diameter may be one of 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, optionally±1, 2, 3, 4, 5, 6, 7, 8, 9 or 10 nm.

The diameter of exosomes ranges from around 30 to around 100 nm. In some cases, a population of exosomes, as may be present in a composition, comprises exosomes with diameters ranging from 10-200 nm, from 10-150 nm, from 10-120 nm, from 10-100 nm, from 20-150 nm, from 20-120 nm, from 25-110 nm, from 25-100 nm, or from 30-100 nm. Preferably, the diameters are from 30-100 nm. A population of exosomes, for example as present in a composition, pharmaceutical composition, medicament or preparation, will comprise exosomes with a range of different diameters, the median diameter of exosomes within a sample can range ranging from 10-200 nm, from 10-150 nm, from 10-120 nm, from 10-100 nm, from 20-150 nm, from 20-120 nm, from 25-110 nm, from 25-100 nm, or from 30-100 nm. Preferably, the median diameter is between 30-100 nm. The mean average diameter may be one of 10 nm, 20 nm, 30 nm, 40 nm, 50 nm, 60 nm, 70 nm, 80 nm, 90 nm, 100 nm, 110 nm, or 120 nm, optionally±1, 2, 3, 4, 5, 6, 7, 8, 9 or 10 nm.

A population of extracellular vesicles may comprise one of at least 10, 100, 1000, 10⁴, 10⁵, 10⁶, 10⁷, 10⁸, 10⁹, 10¹⁰, 10¹¹, 10¹², 10¹³ or 10¹⁴ extracellular vesicles (optionally per ml of carrier).

Exosomes are observed in a variety of cultured cells including lymphocytes, dendritic cells, cytotoxic T cells, mast cells, neurons, oligodendrocytes, Schwann cells, and intestinal epithelial cells. Exosomes originate from the endosomal network that locates in within multivesicular bodies, large sacs in the cytoplasm. These sacs fuse to the plasma membrane, before being released into extracellular environment.

Apoptotic bodies or blebs are the largest extracellular vesicles, ranging from 1-5 μm. Nucleated cells undergoing apoptosis pass through several stages, beginning with condensation of the nuclear chromatin, membrane blebbing and finally release of EVs including apoptotic bodies.

Preferably, the extracellular vesicles are derived from human cells, or cells of human origin. The extracellular vesicles of the invention may have been induced from cells contacted with a vesicle inducing agent. The vesicle inducing agent may be calcium ionophore, lysophosphatidic acid (LPA), or phorbol-12-myristat-13-acetate (PMA).

In many aspects described herein, the cells are not modified. In particular, the cells from which the extracellular vesicles are derived do not comprise exogenous nucleic acid or proteins. In some cases, the cells are ex vivo, such as resulting from a blood draw. In some cases, the cells have not been modified, such as transduced, transfected, infected, or otherwise modified, but are substantially unchanged as compared to the cells in vivo. Where the cells are red blood cells, the cells may contain no DNA, or may contain substantially no DNA. The red blood cells may be DNA free. Accordingly, in preferred embodiments the extracellular vesicles are loaded with their nucleic acid cargo after the extracellular vesicles have been formed and isolated. Preferably, the extracellular vesicles do not contain nucleic acid, particularly DNA, that was present in the cells from which they are derived.

### Red Blood Cell Extracellular Vesicles (RBCEVs)

In certain aspects disclosed herein, the extracellular vesicles are derived from red blood cells (erythrocytes). Red blood cells are a preferred source of EVs for a number of reasons. Because red blood cells are enucleated, RBCEVs contain less nucleic acid than EVs from other sources. RBCEVs do not contain endogenous DNA. RBCEVs may contain miRNAs or other RNAs. RBCEVs are free from oncogenic substances such as oncogenic DNA or DNA mutations. RBCEVs are not exosomes because, as explained above, exosomes are derived from the endosomal network of the cell, including the endosomes and endoplasmic reticulum. Red blood cells lack most cellular organelles, and in particular do not have endosomes and an endoplasmic reticulum, and thus cannot produce exosomes.

In some cases, the EVs are non-exosomal EVs derived from red blood cells, e.g. human red blood cells.

In some cases, the RBCEVs are isolated from RBCs. A method for isolation and characterisation of RBCEVs is described in Usman et al. (Efficient RNA drug delivery using red blood cell extracellular vesicles. Nature Communications 9, 2359 (2018) doi:10.1038/s41467-018-04791-8), incorporated herein in its entirety by reference.

RBCEVs may comprise hemoglobin and/or stomatin and/or flotillin-2. They may be red in colour. Typically RBCEVs exhibit a domed (concave) surface, or “cup shape” under transmission electron microscopes. The RBCEV may be characterised by having cell surface CD235a.

RBCEVs according to the invention may be about 100 nm to about 300 nm in diameter. In some cases, a composition of RBCEVs comprises RBCEVs with diameters ranging from 50-1000 nm, from 50-750 nm, from 50-500 nm, from 50-300 nm, from 50-200 nm, from 50-150 nm, from 101-1000 nm, from 101-750 nm, from 101-500 nm, from 101-300 nm, from 100-300 nm, from 100-200 nm or from 100-150 nm. Preferably, the diameters are from 50-300 nm, from 50-200 nm, from 50-150 nm, 100-300 nm, from 100-200 nm, or from 100-150 nm.

A population of RBCEVs, e.g. as may be present in a composition, will comprise RBCEVs with a range of different diameters, the median diameter of RBCEVs within a RBCEV sample can range from 50-1000 nm, from 50-750 nm, from 50-500 nm, from 50-300 nm, from 50-200 nm, from 50-150 nm, from 101-1000 nm, from 101-750 nm, from 101-500 nm, from 101-300 nm, from 100-300 nm, from 100-200 nm or from 100-150 nm. Preferably, the median diameter is between 50-300 nm, from 50-200 nm, from 50-150 nm, 100-300 nm, from 100-200 nm, or from 100-150 nm. The mean average diameter may be one of 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, optionally±1, 2, 3, 4, 5, 6, 7, 8, 9 or 10 nm.

Preferably, the RBCEVs are derived from a human or animal blood sample or red blood cells derived from primary cells or immobilized red blood cell lines. The blood cells may be type matched to the patient to be treated, and thus the blood cells may be Group A, Group B, Group AB or Group O. Preferably the blood is Group O. The blood may be rhesus positive or rhesus negative. In some cases, the blood is Group O and/or rhesus negative, such as Type O−. The blood may have been determined to be free from disease or disorder, such as free from HIV, HBV, HCV, syphilis, sickle cell anemia, SARS-COV2, and/or malaria. However, any blood type may be used. In some cases, the RBCEVs are autologous and derived from a blood sample obtained from the patient to be treated. In some cases, the RBCEVs are allogenic and not derived from a blood sample obtained from the patient to be treated.

RBCEVs may be isolated from a sample of red blood cells. Protocols for obtaining EVs from red blood cells are known in the art, for example in Danesh et al. (2014) Blood. 2014 Jan 30; 123(5): 687-696. Methods useful for obtaining EVs may include the step of providing or obtaining a sample comprising red blood cells, inducing the red blood cells to produce extracellular vesicles, and isolating the extracellular vesicles. The sample may be a whole blood sample. Preferably, cells other than red blood cells have been removed from the sample, such that the cellular component of the sample is red blood cells.

The red blood cells in the sample may be concentrated, or partitioned from other components of a whole blood sample, such as white blood cells and plasma. Red blood cells may be concentrated by centrifugation. The sample may be subjected to leukocyte reduction.

The sample comprising red blood cells may comprise substantially only red blood cells. Extracellular vesicles may be induced from the red blood cells by contacting the red blood cells with a vesicle inducing agent. The vesicle inducing agent may be calcium ionophore, lysophosphatidic acid (LPA), or phorbol-12-myristat-13-acetate (PMA).

RBCEVs may be isolated by centrifugation (with or without ultracentrifugation), precipitation, filtration processes such as tangential flow filtration, or chromatography (e.g. see Usman et al., supra). In this way, RBCEVs may be separated from RBCs and other components of the mixture.

Extracellular vesicles may be obtained from red blood cells by a method comprising: obtaining a sample of red blood cells; contacting the red blood cells with a vesicle inducing agent; and isolating the induced extracellular vesicles.

The red blood cells may be separated from a whole blood sample containing white blood cells and plasma by low speed centrifugation and using leukodepletion filters. In some cases, the red blood cell sample contains no other cell types, such as white blood cells. In other words, the red blood cell sample consists substantially of red blood cells. The red blood cells may be diluted in buffer such as PBS prior to contacting with the vesicle inducing agent. The vesicle inducing agent may be calcium ionophore, lysophosphatidic acid (LPA) or phorbol-12-myristat-13-acetate (PMA). The vesicle inducing agent may be about 10 nM calcium ionophore. The red blood cells may be contacted with the vesicle inducing agent overnight, or for at least 1, at least 2, at least 3, at least 4, at least 5, at least 6, at least 7, at least 8, at least 9, at least 10, at least 11, at least 12 or more than 12 hours. The red blood cells may be contacted with the vesicle inducing agent at a plurality of time points The mixture may be subjected to low speed centrifugation to remove RBCs, cell debris, or other non-RBCEVs matter and/or passing the supernatant through an about 0.45 μm syringe filter. RBCEVs may be concentrated by ultracentrifugation, such as centrifugation at around 100,000×g. RBCEVs may be concentrated by centrifugation at 10,000×g, 15,000×g, 20,000×g, 25,000×g, 30,000×g, 40,000×g, 50,000×g, 60,000×g, 70,000×g, 80,000×g, 90,000×g or 100,000×g. In certain preferred methods described herein, the RBCEVs are concentrated at between 10,000×g and 50,000×g, or about 15,000×g. The RBCEVs may be concentrated by ultracentrifugation for at least 10 minutes, at least 20 minutes, at least 30 minutes, at least 40 minutes, at least 50 minutes or at least one hour. The concentrated RBCEVs may be suspended in cold PBS. They may be layered on a 60% sucrose cushion. The sucrose cushion may comprise frozen 60% sucrose. The RBCEVs layered on the sucrose cushion may be subject to ultracentrifugation at 100,000×g for at least one hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours or more. Preferably, the RBCEVs layered on the sucrose cushion may be subject to ultracentrifugation at 100,000×g for about 16 hours. The red layer above the sucrose cushion is then collected, thereby obtaining RBCEVs. The obtained RBCEVs may be subject to further processing, such as washing, tagging, and optionally loading.

### Surface Tagging

Extracellular vesicles may comprise a tag, preferably attached to, or inserted through, the vesicle membrane.

The extracellular vesicles may have, at their surface, a tag. The tag is preferably a protein or peptide sequence. The tag may be a peptide or protein. It may be a modified peptide or protein, such as a glycosylated or biotinylated protein or peptide. The tag may be covalently linked to the extracellular vesicle, such as covalently linked to a membrane protein in the extracellular vesicle. The tag may have been added to the extracellular vesicle after the extracellular vesicle had formed. The tag may be linked to the extracellular vesicle by a sequence that comprises or consists of a sequence that is, or that is derived from, a protein ligase recognition sequence. For example, the tag may be linked to the extracellular vesicle by a sequence that comprises 100% sequence identity to a protein ligase recognition sequence, or about 90%, about 80%, about 70%, about 60%, about 50% or about 40% sequence identity to a protein ligase recognition sequence. The amino acid sequence may comprise LPXT.

The tag may be presented on the external surface of the vesicle, and thus exposed to the extravesicular environment.

The tag may be an exogenous molecule. In other words, the tag is a molecule that is not present on the external surface of the vesicle in nature. In some cases, the tag is an exogenous molecule that is not present in the cell or red blood cell from which the extracellular vesicle is derived.

The tag may increase the stability, uptake efficiency and availability in the circulation of the extracellular vesicles.

In some cases, the tag acts to present the extracellular vesicles and extracellular vesicles containing cargoes in the circulation and organs in the body. The peptides and proteins can act as therapeutic molecules such as blocking/activating target cell function or presenting antigens for vaccination. They can also act as probes for biomarker detection such as diagnosis of toxins.

The tag may contain a functional domain and a protein ligase recognition sequence. The functional domain may be capable of binding to a target moiety, capable of detection, or capable of inducing a therapeutic effect. The functional domain may be capable of binding to a target molecule. Tags comprising such a functional domain may be referred to herein as binding molecules. A binding molecule is one that is capable of interacting specifically with a target molecule. Extracellular vesicles comprising a binding moiety may be particularly useful for delivering a cargo or a therapeutic agent to a cell that has the target molecule. Suitable binding molecules include antibodies and antigen binding fragments (sometimes known as antibody fragments), ligand molecules and receptor molecules. The binding molecule will bind to a target of interest. The target may be a molecule associated with, such as expressed on the surface of, a cell of interest. The ligand may form a complex with a nucleic acid on the target cell, such as a receptor molecule. The target may be a molecule associated with an immune cell, such as a cell surface marker. Suitable binding molecules include antibodies and antigen binding fragments.

Other suitable binding molecules include ligands and receptors that have affinity for a target molecule. The tag may be a ligand of a cell surface receptor. Examples include streptavidin and biotin, avidin and biotin, or ligands of other receptors, such as fibronectin and integrin. The small size of biotin results in little to no effect to the biological activity of bound molecules. As biotin and streptavidin, biotin and avidin, and fibronectin and integrin bind their pairs with high affinity and specificity, they are very useful as binding molecules. The Avidin-biotin complex is the strongest known non-covalent interaction (Kd=10⁻¹⁵ M) between a protein and ligand. Bond formation is rapid, and once formed, is unaffected by extremes of pH, temperature, organic solvents and other denaturing agents. The binding of biotin to streptavidin and is also strong, rapid to form and useful in biotechnology applications.

The functional domain may comprise or consist of a therapeutic agent. The therapeutic agent may be an enzyme. It may be an apoptotic inducer or inhibitor.

The functional domain may comprise an antigen or antibody recognition sequence. The tag may comprise one or more short peptides derived from one or more antigenic peptides. The peptide may be a fragment of an antigenic peptide. Suitable antigenic peptides are known to one of skill in the art.

The functional domain may comprise or consist of a detectable moiety. Detectable moieties include fluorescent labels, colorimetric labels, photochromic compounds, magnetic particles or other chemical labels. The detectable moiety may be biotin, a FLAG tag, or a His tag.

The tag may comprise a spacer or linker moiety. The spacer or linker may be arranged between the tag and the protein ligase recognition sequence. The spacer or linker may be linked to the N or C terminus of the tag. The spacer or linker may be arranged so as not to interfere or impede the function of the tag, such as the target binding activity by the tag. The spacer or linker may be a peptide sequence. In some case, the spacer or linker is a series of at least 1, at least 2, at least 3, at least 4, at least 5, at least 6, at least 7, at least 8, at least 9, at least 10 amino acids, at least 11 amino acids, at least 12 amino acids, at least 13 amino acids, at least 14 amino acids or at least 15 amino acids. The spacer or linker may be flexible. The spacer may comprise a plurality of glycine and/or serine amino acids.

Spacer and linker sequences are known to the skilled person, and are described, for example in Chen et al., Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues.

In some cases, the spacer or linker sequence comprises at least one glycine residue and/or at least one serine residue. In some embodiments the linker sequence consists of glycine and serine residues. In some cases, the spacer or linker sequence has a length of 1-2, 1-3, 1-4, 1-5 or 1-10 amino acids.

Inclusion of the spacer or linker may improve the efficiency of the protein ligase reaction between the extracellular vesicle and the tag moiety. The term “tag” as used herein may encompass a peptide comprising a tag, a spacer, and protein ligase recognition sequence.

Suitable protein ligase recognition sequences are known in the art. The protein ligase recognition sequence is recognised by the protein ligase used in the method of tagging the extracellular vesicles. For example, if the protein ligase used in the method is a sortase, then the protein ligase recognition sequence is a sortase binding site. In those cases, the sequence may be LPXTG (where X is any naturally occurring amino acid), preferably LPETG. Alternatively, where the enzyme is Asparaginyl endopeptidase 1 (AEP1), the protein ligase recognition sequence may be NGL. The protein ligase binding site may be arranged at the C terminus of the peptide or protein.

The tag may additionally comprise one or more further sequences to aid in purification or processing of the tag, during production of the tag itself, during the tagging method, or for subsequent purification. Any suitable sequence known in the art may be used. For example, the sequence may be an HA tag, a FLAG tag, a Myc tag, a His tag (such as a poly His tag, or a 6xHis tag).

The tag may be linked to substantially all of the extracellular vesicles in a population or composition. Compositions disclosed herein may comprise extracellular vesicles in which at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, or at least 97% of the extracellular vesicles comprise the tag. Preferably, at least 85%, at least 90%, at least 95%, at least 96% or at least 97% of the extracellular vesicles comprise the tag. In some cases, different extracellular vesicles within the composition comprise different tags. In some cases, the extracellular vesicles comprise the same, or substantially the same, tag.

Methods for incorporating a tag are described in PCT/SG2019/050481, WO 2014/183071 A2, WO 2014/183066 A2 and US 2014/0030697 A1, each incorporated herein by reference in its entirety.

### Cargo

Extracellular vesicles described herein are loaded with, or contain, a cargo. The present disclosure is particularly concerned with a cargo comprising a plurality of non-identical nucleic acids. A nucleic acid is non-identical with respect to another nucleic acid if it differs in at least one characteristic. Examples of such characteristics are described below. The terms “non-identical” and “different” are used interchangeably herein.

As demonstrated herein, extracellular vesicles can be loaded with a heterogenous cargo of nucleic acids. In other words, an extracellular vesicle may comprise at least one first nucleic acid and at least one second nucleic acid. In some cases, an extracellular vesicle may comprise at least one first nucleic acid, at least one second nucleic acid, and at least one third nucleic acid. Each of the first and second, or first, second and third nucleic acids is non-identical. In some cases, the extracellular vesicle may contain more than one copy of the first and second, or first, second and third nucleic acid. However, what is important is that at least one of nucleic acid molecules in the EV is different to at least one of the other nucleic acids in the EV or, in the case of an EV loaded with three different nucleic acids, that at least one of each of three different nucleic acids is present.

A nucleic acid is non-identical with respect to a further nucleic acid where it is a different class of nucleic acid. Classes of nucleic acid include ssDNA, dsDNA, ssRNA, dsRNA, small interfering RNA (siRNA), messenger RNA (mRNA), guide RNA (gRNA), CRISPR RNA (crRNA), trans-activating CRISPR RNA (tracrRNA), prime editing guide RNA (pegRNA), circular RNA, microRNA (miRNA), primary miRNA (pri-miRNA), precursor miRNA (pre-miRNA), piwi-interacting RNA (piRNA), transfer RNA (tRNA), long noncoding RNA (IncRNA), antisense oligonucleotide (ASO), short hairpin RNA (shRNA), small activating RNA (saRNA), small nucleolar RNAS (snoRNA), gapmer, locked nucleic acid (LNA), peptide nucleic acid (PNA), expression vector, circular DNA, linear double stranded DNA, DNA plasmid, RNA plasmid, DNA minicircle, a dumbbell-shaped DNA minimal vector and RNA minicircle.

A nucleic acid may be a DNA. A nucleic acid may be an RNA. A nucleic acid may be a ssDNA. A nucleic acid may be a dsDNA. A nucleic acid may be a ssRNA. A nucleic acid may be a dsRNA. A nucleic acid may be a small interfering RNA (SIRNA). A nucleic acid may be a messenger RNA (mRNA). A nucleic acid may be a guide RNA (gRNA). A nucleic acid may be a CRISPR RNA (crRNA). A nucleic acid may be a trans-activating CRISPR RNA (tracrRNA). A nucleic acid may be a prime editing guide RNA (pegRNA). A nucleic acid may be a circular RNA. A nucleic acid may be a microRNA (miRNA). A nucleic acid may be a primary miRNA (pri-miRNA). A nucleic acid may be a precursor miRNA (pre-miRNA). A nucleic acid may be a piwi-interacting RNA (piRNA). A nucleic acid may be a transfer RNA (tRNA). A nucleic acid may be a long noncoding RNA (IncRNA). A nucleic acid may be an antisense oligonucleotide (ASO). A nucleic acid may be a short hairpin RNA (shRNA). A nucleic acid may be a small activating RNA (saRNA). A nucleic acid may be a small nucleolar RNAs (snoRNA). A nucleic acid may be a gapmer. A nucleic acid may be a locked nucleic acid (LNA). A nucleic acid may be a peptide nucleic acid (PNA). A nucleic acid may be an expression vector. A nucleic acid may be a DNA plasmid. A nucleic acid may be an RNA plasmid. A nucleic acid may be a DNA minicircle. A nucleic acid may be dumbbell-shaped DNA minimal vector. A nucleic acid may be an RNA minicircle.

The cargo may comprise two or more nucleic acids, wherein two or more of the nucleic acids are different types of nucleic acid. The cargo may comprise 3 or more nucleic acids, wherein three or more of the nucleic acids are of different types. The cargo may comprise two or more nucleic acids of the same type, wherein two or more of the nucleic acids are not identical. The cargo may comprise a plurality of DNA molecules. The cargo may comprise a plurality of RNA molecules. The cargo may comprise a plurality of plasmids. The cargo may comprise a plurality of minicircles. The cargo may comprise a plurality of dumbbell-shaped DNA minimal vectors. The cargo may comprise a plurality of mRNA molecules. The cargo may comprise a plurality of expression vectors. The cargo may comprise a plurality of nucleic acids, wherein two or more of the nucleic acids is each independently selected from the group consisting of ssDNA, dsDNA, ssRNA, dsRNA, small interfering RNA (siRNA), messenger RNA (mRNA), guide RNA (gRNA), CRISPR RNA (crRNA), trans-activating CRISPR RNA (tracrRNA), prime editing guide RNA (pegRNA), circular RNA, microRNA (miRNA), primary miRNA (pri-miRNA), precursor miRNA (pre-miRNA), piwi-interacting RNA (piRNA), transfer RNA (tRNA), long noncoding RNA (IncRNA), antisense oligonucleotide (ASO), short hairpin RNA (shRNA), small activating RNA (saRNA), small nucleolar RNAS (snoRNA), gapmer, locked nucleic acid (LNA), peptide nucleic acid (PNA), expression vector, a circular DNA, a double stranded linear DNA, DNA plasmid, RNA plasmid, DNA minicircle and RNA minicircle. For example, the cargo may comprise a DNA and an RNA. The cargo may comprise a plasmid and a gRNA. The cargo may comprise a minicircle and a gRNA. The cargo may comprise an expression vector and a gRNA. The cargo may comprise an mRNA and a gRNA. The cargo may comprise dsDNA and a gRNA. The cargo may comprise a plasmid and a pegRNA. The cargo may comprise a minicircle and a pegRNA. The cargo may comprise an expression vector and a pegRNA. The cargo may comprise an mRNA and a pegRNA. The cargo may comprise dsDNA and a pegRNA. The cargo may comprise a plasmid and a minicircle. The cargo may comprise a plasmid and an expression vector. The cargo may comprise a plasmid and an mRNA. The cargo may comprise a plasmid and a dsDNA. The cargo may comprise a minicircle and an expression vector. The cargo may comprise a minicircle and mRNA. The cargo may comprise a minicircle and a dsDNA. The cargo may comprise an expression vector and an mRNA. The cargo may comprise an expression vector and a dsDNA. The cargo may comprise an mRNA and a dsDNA. The cargo may comprise a plasmid and an antisense oligonucleotide. The cargo may comprise a plasmid and an siRNA. The cargo may comprise a plasmid, a gRNA and a dsDNA. The cargo may comprise a minicircle, a gRNA and a plasmid. The cargo may comprise a minicircle, a gRNA and a dsDNA. The cargo may comprise an expression vector, a gRNA and a plasmid. The cargo may comprise an expression vector, a gRNA and a dsDNA. The cargo may comprise an mRNA, a gRNA and a plasmid. The cargo may comprise an mRNA, a gRNA and a dsDNA.

In some aspects described herein, the cargo comprises at least two non-identical nucleic acids. A nucleic acid is non-identical with respect to a further nucleic acid where it is a different size, length (i.e. has a different number of bases or base pairs), where it has a different sequence, or where it comprises modified bases at different positions in the sequence to the further nucleic acid. The nucleic acids are discrete molecules. The at least two nucleic acids or at least three nucleic acids are not contiguous or continuous nucleic acid sequences that combined form a larger nucleic acid. The cargo may comprise at least 2 nucleic acids having different lengths (i.e. different numbers of bases or base pairs). The cargo may comprise at least 2 nucleic acids having the same length. The cargo may comprise at least 2 nucleic acids, wherein one of the nucleic acids is a single stranded molecule and one of the nucleic acids is double stranded. In some cases, both nucleic acids are single stranded or both nucleic acids are double stranded, but differ in other ways such as size, length or sequence. A nucleic acid may be non-identical with respect to a further nucleic acid where it comprises a different nucleotide sequence. The cargo may comprise at least 2 nucleic acids having different sequences. The cargo may comprise at least 2 nucleic acids having the same sequence, but different in base modifications, type of nucleic acid, or double/single strandedness. A nucleic acid may be non-identical with respect to a further nucleic acid where it encodes a different product type (e.g. a peptide/polypeptide of interest, a, gRNA, an siRNA, or an ASO) or a product having a different sequence (e.g. an RNA having a different nucleotide sequence or a peptide or protein having a different amino acid sequence). The cargo may comprise at least 2 nucleic acids encoding different products. The cargo may comprise at least 2 nucleic acids encoding the same product but having non-identical sequences. The cargo may comprise at least 2 nucleic acids encoding products having different sequences. The cargo may comprise at least 2 nucleic acids encoding products having the same sequence, wherein the nucleic acids have different sequences. A nucleic acid is non-identical with respect to a further nucleic acid where it comprises one or more modified nucleotides or other modification which is not present, or which is present at a different position, in the further nucleic acid. The cargo may comprise at least 2 nucleic acids having different modifications. The cargo may comprise at least 2 nucleic acids having the same modification.

### Nucleic Acids

A nucleic acid cargo refers to a nucleic acid (e.g. oligonucleotide or polynucleotide) loaded into or onto an extracellular vesicle. A nucleic acid cargo normally refers to an oligonucleotide strand (which may be in any form, e.g. single stranded, double stranded, super-coiled or not super-coiled, chromosomal or non-chromosomal). The nucleic acid may be conjugated to, or complexed with, other molecules, e.g. carriers, stabilisers, histones, lipophilic agents.

Methods disclosed herein may be used for any nucleic acid cargo. Nucleic acid may be double or single stranded. The nucleic acid may be circular.

The nucleic acid cargo may be a minicircle. Minicircles are small (around 4 kbp) circular replicons. Minicircles usually comprise DNA, normally double stranded. Although minicircles occur naturally in some eukaryotic organelle genomes, minicircles preferred herein are synthetically derived. In some cases, the minicircle does not comprise an origin of replication, and thus does not replicate within the cell. Minicircles are known to those of ordinary skill in the art, e.g. see Gaspar et al., Minicircle DNA vectors for gene therapy: advances and applications. Expert Opin Biol Ther 2015 Mar; 15(3):353-79. doi: 10.1517/14712598.2015.996544. Epub 2014 Dec 24, incorporated by reference in its entirety herein. In some cases the minicircle comprises a reporter gene.

The nucleic acid may be a dumbbell-shaped DNA minimal vector. Dumbbell-shaped DNA minimal vectors are described in Yu et al (Nucleic Acids Research 2015: 43(18): e120), Jiang et al (Molecular Therapy 2016: 24(9): 1581-1591) and Zanta et al (PNAS 1999: 96: 91-96), each incorporated herein by reference in its entirety. A dumbbell-shaped DNA minimal vector comprises a DNA oligonucleotide that has a secondary structure comprising one or more hairpins.

In some cases, the nucleic acid cargo is a plasmid. A plasmid is normally able to replicate independently in a cell. The plasmid may comprise an origin of replication sequence.

In some cases the nucleic acid is not modified to contain a sequence that binds to a protein on the surface of the vesicle. For example, the cargo nucleic acid does not contain a trans activating response (TAR) element. In some cases, the extracellular vesicle is not modified to contain a modified surface protein, such as an exogenous ARRDC1 protein or sequence derived from an ARRDC1 protein.

The cargo is preferably exogenous. In other words, the nucleic acid is not present in the extracellular vesicles when they are newly generated, and/or in the cells from which the extracellular vesicles are derived. The cargo may be synthetic, having been designed and/or constructed in vitro or in silico.

The cargo may be a therapeutic oligonucleotide or a diagnostic oligonucleotide. The cargo may exert a therapeutic effect in a target cell after being delivered to that target cell. The nucleic acid may encode a gene of interest. For example, the cargo may encode a functional gene to replace an absent gene, repair a defective gene, or induce a therapeutic effect in a target tissue. In some cases, the cargo is a reporter gene or encodes a molecule that is readily detectable.

In some cases, the cargo may be a nucleic acid. The nucleic acid may be single stranded or double stranded. The cargo may be an RNA. The RNA may be a therapeutic RNA. The RNA may be a small interfering RNA (siRNA), a messenger RNA (mRNA), a guide RNA (gRNA), a prime editing guide RNA (pegRNA), a circular RNA, a microRNA (miRNA), a piwiRNA (piRNA), a transfer RNA (tRNA), or a long noncoding RNA (IncRNA) produced by chemical synthesis or in vitro transcription. In some cases, the cargo is an antisense oligonucleotide, for example, having a sequence that is complementary to an endogenous nucleic acid sequence such as a transcription factor, miRNA or other endogenous mRNA.

The cargo may be, or may encode, a molecule of interest. For example, the cargo may be an mRNA that encodes Cas9 or another nuclease. The cargo may encode one or more peptides/polypeptides of interest. The cargo may encode an antigen-binding molecule or fragment thereof.

In some cases, the cargo is a nucleic acid that is, or that encodes, an siRNA or antisense oligonucleotide (ASO). Such cargo may be useful in methods of gene silencing or downregulating gene expression. The siRNA or ASO may correspond to a sequence that is expressed in a target cell, e.g. an mRNA sequence. It may act to inhibit or enhance the expression of a particular gene or protein of interest. The nucleic acid may encode an siRNA or ASO corresponding to a miRNA expressed in a target cell.

The cargo may comprise or encode an mRNA. The mRNA may encode a transgene.

In the cell, an antisense nucleic acid may hybridize to the corresponding mRNA, forming a double-stranded molecule. The antisense nucleic acids may interfere with the translation of the mRNA, since the cell will not translate an mRNA that is double-stranded. The use of antisense methods to inhibit the in vitro translation of genes is well known in the art (see e.g. Marcus-Sakura, Anal. Biochem. 1988, 172:289). Further, antisense molecules which bind directly to the DNA may be used. Antisense nucleic acids may be single or double stranded nucleic acids. Non-limiting examples of antisense nucleic acids include small interfering RNA (siRNA; including their derivatives or pre-cursors, such as nucleotide analogs), short hairpin RNAs (shRNA), micro RNAs (miRNA), saRNAs (small activating RNAs), small nucleolar RNAs (snoRNA) or certain of their derivatives or pre-cursors, long non-coding RNA (IncRNA), or single stranded molecules such as chimeric ASOs or gapmers. Antisense nucleic acid molecules may stimulate RNA interference (RNAi) or other cellular degradation mechanisms such as RNase degradation.

In some preferred embodiments, the cargo comprises or encodes an ASO that targets, e.g. hybridises to, a micro RNA. In some cases the ASO inhibits the function of the micro RNA and prevents the miRNA from post-transcriptionally regulating gene expression. In some cases the ASO functions to upregulate expression of one or more genes that are usually downregulated by a miRNA. Thus, an antisense nucleic acid cargo may interfere with transcription of target genes, interfere with translation of target mRNA and/or promote degradation of target mRNA. In some cases, an antisense nucleic acid is capable of inducing a reduction in expression of the target gene.

A “siRNA,” “small interfering RNA,” “small RNA,” or “RNAi” as provided herein, refers to a nucleic acid that forms a double stranded RNA, which double stranded RNA has the ability to reduce or inhibit expression of a gene or target gene when expressed in the same cell as the gene or target gene. The complementary portions of the nucleic acid that hybridize to form the double stranded molecule typically have substantial or complete identity. In one embodiment, a siRNA or RNAi is a nucleic acid that has substantial or complete identity to a target gene and forms a double stranded siRNA. In embodiments, the SiRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In some embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

RNAi and siRNA are described in, for example, Dana et al., Int J Biomed Sci. 2017; 13(2): 48-57, herein incorporated by reference in its entirety. An antisense nucleic acid molecule may contain double-stranded RNA (dsRNA) or partially double-stranded RNA that is complementary to a target nucleic acid sequence. A double-stranded RNA molecule is formed by the complementary pairing between a first RNA portion and a second RNA portion within the molecule. The length of an RNA sequence (i.e. one portion) is generally less than 30 nucleotides in length (e.g. 29, 28, 27, 26, 25, 24, 23, 22, 21, 20, 19, 18, 17, 16, 15, 14, 13, 12, 11, 10 or fewer nucleotides). In some embodiments, the length of an RNA sequence is 18 to 24 nucleotides in length. In some siRNA molecules, the complementary first and second portions of the RNA molecule form the “stem” of a hairpin structure. The two portions can be joined by a linking sequence, which may form the “loop” in the hairpin structure. The linking sequence may vary in length and may be, for example, 5, 6, 7, 8, 9, 10, 11, 12, or 13 nucleotides in length. Suitable linking sequences are known in the art.

Suitable siRNA molecules for use in the methods of the present invention may be designed by schemes known in the art, see for example Elbashire et al., Nature, 2001 411:494-8; Amarzguioui et al., Biochem. Biophys. Res. Commun. 2004 316(4):1050-8; and Reynolds et al., Nat. Biotech. 2004, 22(3):326-30. Details for making siRNA molecules can be found in the websites of several commercial vendors such as Ambion, Dharmacon, GenScript, Invitrogen and OligoEngine. The sequence of any potential siRNA candidate generally can be checked for any possible matches to other nucleic acid sequences or polymorphisms of nucleic acid sequence using the BLAST alignment program (see the National Library of Medicine internet website). Typically, a number of siRNAs are generated and screened to obtain an effective drug candidate, see, U.S. Pat. No. 7,078,196. siRNAs can be expressed from a vector and/or produced chemically or synthetically. Synthetic RNAi can be obtained from commercial sources, for example, Invitrogen (Carlsbad, Calif.). RNAi vectors can also be obtained from commercial sources, for example, Invitrogen.

The nucleic acid molecule may be, comprise, or encode a miRNA. The term “miRNA” is used in accordance with its plain ordinary meaning and refers to a small non-coding RNA molecule capable of post-transcriptionally regulating gene expression. In one embodiment, a miRNA is a nucleic acid that has substantial or complete identity to a target gene. In some embodiments, the miRNA inhibits gene expression by interacting with a complementary cellular mRNA thereby interfering with the expression of the complementary mRNA. Typically, the miRNA is at least about 15-50 nucleotides in length (e.g., each complementary sequence of the miRNA is 15-50 nucleotides in length, and the miRNA is about 15-50 base pairs in length). In some cases, the nucleic acid is synthetic or recombinant. The miRNA may be miR-29a. In some cases the nucleic acid is a miRNA stem-loop.

Nucleic acids useful in the methods of the invention include antisense oligonucleotides, mRNA, or siRNAs that target oncogenic miRNAs (also known as oncomiRs) or transcription factors. The cargo may be a ribozyme or aptamer. In some cases, the nucleic acid is a plasmid.

In certain aspects described herein, the cargo is an antisense oligonucleotide (ASO). The antisense oligonucleotide may be complementary to a miRNA or mRNA. The antisense oligonucleotide comprises at least a portion which is complementary in sequence to a target mRNA sequence. The antisense oligonucleotide may bind to, and thereby inhibit, the target sequence. For example, the antisense oligonucleotide may inhibit the translation process of the target sequence. The miRNA may be a miRNA associated with cancer (Oncomir). The miRNA may be miR-125b.

### Gene Editing

In some aspects, the cargo is one or more components of a gene editing system. In some aspects, the cargo is two or more components of a gene editing system. For example, a CRISPR/Cas gene editing system. For example, the cargo may include a nucleic acid which recognises a particular target sequence. The cargo may comprise a gRNA. The cargo may comprise a pegRNA. Such gRNAs and pegRNAs may be useful in CRISPR/Cas gene editing. The cargo may comprise a Cas mRNA or a plasmid encoding Cas. The cargo may comprise a gRNA, and a Cas mRNA or a plasmid encoding Cas. The cargo may comprise a pegRNA and a Cas mRNA or a plasmid encoding Cas. The Cas nuclease may be a Cas9 or a Cas12 nuclease.

Other gene editing molecules may be used as cargo, such as zinc finger nucleases (ZFNs) or Transcription activator-like effector nucleases (TALENs). The cargo may comprise a sequence engineered to target a particular nucleic acid sequence in a target cell. The gene editing molecule may specifically target a miRNA. For example, the gene editing molecule may be a gRNA that targets miR-125b.

In some embodiments the methods employ target gene editing using site-specific nucleases (SSNs). Gene editing using SSNs is reviewed e.g. in Eid and Mahfouz, Exp Mol Med. 2016 Oct; 48(10): e265, which is hereby incorporated by reference in its entirety. Enzymes capable of creating site-specific double strand breaks (DSBs) can be engineered to introduce DSBs to target nucleic acid sequence(s) of interest. DSBs may be repaired by either error-prone non-homologous end-joining (NHEJ), in which the two ends of the break are rejoined, often with insertion or deletion of nucleotides. Alternatively DSBs may be repaired by homology-directed repair (HDR), in which a DNA template with ends homologous to the break site is supplied and introduced at the site of the DSB.

SSNs capable of being engineered to generate target nucleic acid sequence-specific DSBs include ZFNs, TALENs and clustered regularly interspaced palindromic repeats/CRISPR-associated-9 (CRISPR/Cas9) systems.

ZFN systems are reviewed e.g. in Umov et al., Nat Rev Genet. (2010) 11(9):636-46, which is hereby incorporated by reference in its entirety. ZFNs comprise a programmable Zinc Finger DNA-binding domain and a DNA-cleaving domain (e.g. a Fokl endonuclease domain). The DNA-binding domain may be identified by screening a Zinc Finger array capable of binding to the target nucleic acid sequence. ZFNs work in pairs as the endonuclease (e.g. Fokl) functions as a dimer. A ZFN system comprises two monomers with unique DNA recognition sites in the target genome with proper orientation (i.e. on opposite DNA strands) and spacing to allow the endonuclease to function.

In some aspects the EV is loaded with a ZFN system related cargo. In these aspects, the EV is useful in a method of gene editing.

The cargo may be a nucleic acid encoding components of a ZFN gene-editing system. A ZFN gene editing system may comprise a ZFN pair having two polypeptide monomers. The monomers may be encoded by the same nucleic acid molecule. The monomers may be encoded by separate nucleic acid molecules. The nucleic acid encoding the monomer(s) may be DNA. The nucleic acid encoding the monomer(s) may be a plasmid. The nucleic acid encoding the monomer(s) may be an expression vector. The nucleic acid encoding the monomer(s) may be an mRNA. The nucleic acid encoding the monomer(s) may be a minicircle. The nucleic acid encoding the monomer(s) may be a dumbbell-shaped DNA minimal vector.

In some aspects described herein, the cargo comprises a first nucleic acid molecule that encodes a first monomer of a ZFN pair and a further nucleic acid molecule that encodes a second monomer of a ZFN pair. The nucleic acids may comprise an expression cassette such that the ZFN monomers are expressed within a target cell. The expressed ZFN monomers may then bind to their respective DNA recognition sites to allow dimerization of the endonuclease. The endonuclease can then function to introduce a double strand break into the DNA.

TALEN systems are reviewed e.g. in Mahfouz et al., Plant Biotechnol J. (2014) 12(8): 1006-14, which is hereby incorporated by reference in its entirety. TALENs comprise a programmable DNA-binding TALE domain and a DNA-cleaving domain (e.g. a Fokl endonuclease domain). TALEs comprise repeat domains consisting of repeats of 33-39 amino acids, which are identical except for two residues at positions 12 and 13 of each repeat which are repeat variable di-residues (RVDs). Each RVD determines binding of the repeat to a nucleotide in the target DNA sequence according to the following relationship: “HD” binds to C, “NI” binds to A, “NG” binds to T and “NN” or “NK” binds to G (Moscou and Bogdanove, Science (2009) 326(5959):1501.).

TALENs work in pairs as the endonuclease (e.g. Fokl) functions as a dimer. A TALEN system comprises two monomers with unique DNA recognition sites in the target genome with proper orientation (i.e. on opposite DNA strands) and spacing to allow the endonuclease to function.

In some aspects the EV is loaded with a TALEN system related cargo. In these aspects, the EV is useful in a method of gene editing.

The cargo may be a nucleic acid encoding components of a TALEN gene editing system. A TALEN gene editing system may comprise a TALEN pair having two polypeptide monomers. The monomers may be encoded by the same nucleic acid molecule. The monomers may be encoded by separate nucleic acid molecules. The nucleic acid encoding the monomer(s) may be DNA. The nucleic acid encoding the monomer(s) may be a plasmid. The nucleic acid encoding the monomer(s) may be an expression vector. The nucleic acid encoding the monomer(s) may be an mRNA. The nucleic acid encoding the monomer(s) may be a minicircle. The nucleic acid encoding the monomer(s) may be a dumbbell-shaped DNA minimal vector.

In some aspects described herein, the cargo comprises a first nucleic acid molecule that encodes a first monomer of a TALEN pair and a further nucleic acid molecule that encodes a second monomer of a TALEN pair. The nucleic acids may comprise an expression cassette such that the TALEN monomers are expressed within a target cell. The expressed TALEN monomers may then bind to their respective DNA recognition sites to allow dimerization of the endonuclease. The endonuclease can then function to introduce a double strand break into the DNA.

CRISPR is an abbreviation of Clustered Regularly Interspaced Short Palindromic Repeats. The term was first used at a time when the origin and function of these sequences were not known and they were assumed to be prokaryotic in origin. CRISPR are segments of DNA containing short, repetitive base sequences in a palindromic repeat (the sequence of nucleotides is the same in both directions). Each repetition is followed by short segments of spacer DNA from previous integration of foreign DNA from a virus or plasmid. Small clusters of CAS (CRISPR-associated) genes are located next to CRISPR sequences. RNA harboring the spacer sequence helps Cas (CRISPR-associated) proteins recognize and cut foreign pathogenic DNA. Other RNA-guided Cas proteins cut foreign RNA. A simple version of the CRISPR/Cas system, CRISPR/Cas9, has been modified to edit genomes. By delivering the Cas9 nuclease and a synthetic guide RNA (gRNA) into a cell, the cell's genome can be cut at a desired location, allowing existing genes to be removed and/or new ones added. CRISPR-Cas systems fall into two classes. Class 1 systems use a complex of multiple Cas proteins to degrade foreign nucleic acids. Class 2 systems use a single large Cas protein for the same purpose. Class 1 is divided into types I, III, and IV; class 2 is divided into types II, V, and VI. CRISPR genome editing uses a type II CRISPR system.

In some aspects, the EV is loaded with a CRISPR related cargo. In other words, the EV is useful in a method involving gene editing, such as therapeutic gene editing. In some cases, the EV is useful for in vitro gene editing. In some cases, the EV is useful for in vivo gene editing.

The cargo may comprise a guide RNA. The guide RNA may comprise a CRISPR RNA (crRNA) and a trans-activating CRISPR RNA (tracrRNA). The crRNA contains a guide RNA that locates the correct section of host DNA along with a region that binds to tracrRNA forming an active complex. The tracrRNA binds to crRNA and forms the active complex. The gRNA combines both the tracrRNA and a crRNA, thereby encoding an active complex. The gRNA may comprise multiple crRNAs and tracrRNAs. The gRNA may be designed to bind to a sequence or gene of interest. The gRNA may target a gene for cleavage. Optionally, an optional section of DNA repair template is included. The repair template may be utilized in either non-homologous end joining (NHEJ) or homology directed repair (HDR).

The cargo may be a nuclease, such as a Cas9 nuclease. The nuclease is a protein whose active form is able to modify DNA. Nuclease variants are capable of single strand nicking, double strand break, DNA binding or other different functions. The nuclease recognises a DNA site, allowing for site specific DNA editing. The nuclease may be modified. For example, the nuclease may be fused to a reverse transcriptase. Such a nuclease may be useful in a prime editing system. In another example the nuclease may be catalytically inactive. Such a nuclease may be fused to a transcription factor and may be useful in systems to regulate transcription.

The gRNA and nuclease may be encoded on a plasmid. In other words, the EV cargo may comprise a plasmid that encodes both the gRNA and the nuclease. The gRNA and nuclease may be encoded on separate plasmids. In other words, the EV cargo may comprise a first plasmid that encodes the gRNA and a further plasmid that encodes the nuclease. In some cases, an EV contains the gRNA and another EV contains or encodes the nuclease. In some cases, an EV contains a plasmid encoding the gRNA, and a plasmid encoding the nuclease. Thus, in some aspects, a composition is provided comprising EVs, wherein a portion of the EVs comprise or encode the nuclease such as Cas9, and a portion of the EVs comprise or encode the gRNA. In some cases, a composition containing EVs that comprise or encode the gRNA and a composition containing EVs that encode or contain the nuclease are co-administered. In some cases, the composition comprises EVs wherein the EVs contain an oligonucleotide that encodes both a gRNA and a nuclease.

The cargo may further comprise a DNA repair template. The DNA repair template may be a linear dsDNA. The DNA repair template may be a plasmid. The DNA repair template may be present on the same plasmid as that encoding the gRNA and/or nuclease. The DNA repair template may be present on a separate plasmid as that encoding the gRNA and/or nuclease. In some cases an EV cargo may comprise a plasmid that encodes both the gRNA and the nuclease and a separate plasmid or linear dsDNA comprising the DNA repair template. In some cases an EV cargo may comprise a first plasmid that encodes the gRNA, a second plasmid that encodes the nuclease and a further plasmid or dsDNA comprising the DNA repair template.

CRISPR/Cas9 and related systems e.g. CRISPR/Cpf1, CRISPR/C2c1, CRISPR/C2c2 and CRISPR/C2c3 are reviewed e.g. in Nakade et al., Bioengineered (2017) 8(3):265-273, which is hereby incorporated by reference in its entirety. These systems comprise an endonuclease (e.g. Cas9, Cpf1 etc.) and the single-guide RNA (sgRNA) molecule. The sgRNA can be engineered to target endonuclease activity to nucleic acid sequences of interest.

### Modified RNA

In some cases, the nucleic acid cargo comprises one or more modified nucleotides or other modifications. Chemical modifications may include chemical substitution at a sugar position, a phosphate position, and/or a base position of the nucleic acid including, for example., incorporation of a modified nucleotide, incorporation of a capping moiety (e.g. 3′ capping), conjugation to a high molecular weight, non-immunogenic compound (e.g. polyethylene glycol (PEG)), conjugation to a lipophilic compound, substitutions in the phosphate backbone. For example, the nucleic acid may comprise one or more 2′-position sugar modifications, such as 2′-amino (2′-NH), 2′-fluoro (2′-F), and 2′-O-methyl (2′-OMe). Base modifications may include 5-position pyrimidine modifications, 8-position purine modifications, modifications at exocyclic amines, substitution of 4-thiouridine, substitution of 5-bromo- or 5-iodo-uracil, backbone modifications, methylations, unusual base-pairing combinations such as the isobases isocytidine and isoguanidine. Modifications can also include 3′ and 5′ modifications, such as capping. Other modifications can include substitution of one or more of the naturally occurring nucleotides with an analog, internucleotide modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present in a sugar may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, or organic capping group moieties of from about 1 to about 20 polyethylene glycol (PEG) polymers or other hydrophilic or hydrophobic biological or synthetic polymers. Nucleic acids may be of variant types, such as locked nucleic acid (LNA), peptide nucleic acid (PNA), or gapmer.

### DNA

A nucleic acid cargo may comprise DNA molecules.

The cargo may comprise an expression vector or expression cassette sequence. Suitable expression vectors and expression cassettes are known in the art. Expression vectors useful in the methods described herein comprise elements that facilitate the expression of one or more nucleic acid sequences in a target cell. Expression vectors useful in the present disclosure may comprise a transgene or other nucleic acid sequence.

An expression vector refers to an oligonucleotide molecule used as a vehicle to transfer foreign genetic material into a cell for expression in/by that cell. Such vectors may include a promoter sequence operably linked to the nucleotide sequence encoding the gene sequence to be expressed. A vector may also include a termination codon and expression enhancers. Any suitable promoters, enhancers and termination codons known in the art may be used.

In this specification the term “operably linked” may include the situation where a selected nucleotide sequence and regulatory nucleotide sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleotide sequence if the regulatory sequence is capable of effecting transcription of the nucleotide sequence. Where appropriate, the resulting transcript may then be translated into a desired protein, peptide or polypeptide.

The cargo may comprise a plurality of expression vectors encoding for different peptides or proteins. The different peptides or proteins may be interrelated, such as subunits or components of the same molecule, or molecules that have an interlinked operation, such as components of the same biological pathways, or exhibit a ligand:receptor binding relationship.

Cargo may comprise a first expression vector encoding a first protein of a protein complex and a further expression vector encoding a further protein of the protein complex. The further protein may be non-identical to the first protein. The cargo may comprise a first expression vector encoding a first domain of a protein and a further expression vector encoding a further domain of a protein. The cargo may comprise a first expression vector encoding a first segment of a protein and a further expression vector encoding a further segment of a protein. For example, the expression vectors may encode different segments of a split protein.

### Vectorised Antibodies

In some aspects, the cargo is one or more components of a vectorized antibody, or antibody gene therapy, system. For example, the cargo may include a nucleic acid which encodes an antigen-binding molecule or fragment thereof.

An antigen-binding molecule refers to a molecule which is capable of binding to a target antigen. Types of antigen-binding molecules include monoclonal antibodies, polyclonal antibodies, monospecific antibodies, multispecific antibodies (e.g., bispecific antibodies), and antibody fragments (e.g. Fv, scFv, Fab, scFab, F(ab′)₂, Fab₂, diabodies, triabodies, scFv-Fc, minibodies, single domain antibodies (e.g. VhH), etc.), as long as they display binding to the relevant target molecule(s). Any of the antibodies or antigen binding fragments disclosed herein may be useful in a vectorised antibody system.

Antibody gene therapy seeks to administer nucleic acids encoding an antibody of interest to the subject. The subject's own cells will then produce and secrete the encoded antibody.

In some aspects, the EV is loaded with a vectorised antibody related cargo. In these aspects, the EV may useful in a method of antibody gene therapy.

The cargo may be a nucleic acid encoding the antigen binding-molecule or a fragment thereof. The nucleic acid encoding the antigen binding-molecule or a fragment thereof may be DNA. The nucleic acid encoding the antigen binding-molecule or a fragment thereof may be a plasmid. The nucleic acid encoding the antigen binding-molecule or a fragment thereof may be an expression vector. The nucleic acid encoding the antigen binding-molecule or a fragment thereof may be an mRNA. The nucleic acid encoding the antigen binding-molecule or a fragment thereof may be a minicircle. The nucleic acid encoding the antigen binding-molecule or a fragment thereof may be a dumbbell-shaped DNA minimal vector

An antigen-binding molecule may be, or may comprise, an antigen-binding polypeptide, or an antigen-binding polypeptide complex. An antigen-binding molecule may comprise more than one polypeptide which together form an antigen-binding domain. The polypeptides may associate covalently or non-covalently. For example, one polypeptide may comprise a heavy chain of an antibody and a further polypeptide may comprise a light chain of an antibody. In another example, one polypeptide may comprise a heavy chain variable region of an antibody and a further polypeptide may comprise a light chain variable region of an antibody. The polypeptides may be encoded by separate nucleic acid molecules.

In some aspects described herein, the cargo comprises a first nucleic acid molecule that encodes a heavy chain of an antibody and a further nucleic acid molecule that encodes the light chain of an antibody. The nucleic acids may comprise an expression cassette such that the heavy chain and the light chain are expressed within a target cell. The expressed proteins may then associate within the cell to form an antibody.

### Antibodies

Antibodies and antigen binding fragments of antibodies are useful in several contexts relating to the present invention. They may be useful as a Tag. They may be useful as cargo. In some cases, they may be encoded by the cargo. In these aspects, a variety of antibodies and antigen binding fragments are relevant.

The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799).

The antibody or antigen binding fragment may be humanised. Methods of humanising antibodies are known in the art, and generally involve the fusing of variable domains of rodent origin to constant domains of human origin such that the resultant antibody retains the antigenic specificity of the rodent parented antibody (Morrison et al (1984) Proc. Natl. Acad. Sd. USA 81, 6851-6855).

Monoclonal antibodies (mAbs) are useful in the methods of the invention and are a homogenous population of antibodies specifically targeting a single epitope on an antigen. Suitable monoclonal antibodies can be prepared using methods well known in the art (e.g. see Köhler, G.; Milstein, C. (1975). “Continuous cultures of fused cells secreting antibody of predefined specificity”. Nature 256 (5517): 495; Siegel DL (2002). “Recombinant monoclonal antibody technology”. Schmitz U, Versmold A, Kaufmann P, Frank HG (2000); “Phage display: a molecular tool for the generation of antibodies—a review”. Placenta. 21 Suppl A: S106-12. Helen E. Chadd and Steven M. Chamow; “Therapeutic antibody expression technology,” Current Opinion in Biotechnology 12, no. 2 (April 1, 2001): 188-194; McCafferty, J.; Griffiths, A.; Winter, G.; Chiswell, D. (1990). “Phage antibodies: filamentous phage displaying antibody variable domains”. Nature 348 (6301): 552-554; “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimaeric antibodies are discussed by Neuberger et al (1988, 8th International Biotechnology Symposium Part 2, 792-799)).

Polyclonal antibodies are useful in the methods of the invention. Monospecific polyclonal antibodies are preferred. Suitable polyclonal antibodies can be prepared using methods well known in the art.

Fragments, such as Fab and Fab2 fragments may be used as can genetically engineered antibodies and antibody fragments. The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855). Antibodies or antigen binding fragments useful in the extracellular vesicles disclosed herein will recognise and/or bind to, a target molecule.

That antigenic specificity is conferred by variable domains and is independent of the constant domains is known from experiments involving the bacterial expression of antibody fragments, all containing one or more variable domains. These molecules include Fab-like molecules (Better et al. (1988) Science 240, 1041); Fv molecules (Skerra et al. (1988) Science 240, 1038); single-chain Fv (ScFv) molecules where the VH and VL partner domains are linked via a flexible oligopeptide (Bird et al. (1988) Science 242, 423; Huston et al. (1988) Proc. Natl. Acad. Sd. USA 85, 5879) and single domain antibodies (dAbs) comprising isolated V domains (Ward et al. (1989) Nature 341, 544). A general review of the techniques involved in the synthesis of antibody fragments which retain their specific binding sites is to be found in Winter & Milstein (1991) Nature 349, 293-299. Antibodies and fragments useful herein may be human or humanized, murine, camelid, chimeric, or from any other suitable source.

By “ScFv molecules” we mean molecules wherein the VH and VL partner domains are covalently linked, e.g. directly, by a peptide or by a flexible oligopeptide. Fab, Fv, ScFv and sdAb antibody fragments can all be expressed in and secreted from E. coli, thus allowing the facile production of large amounts of the said fragments.

Whole antibodies, and F(ab′)2 fragments are “bivalent”. By “bivalent” we mean that the said antibodies and F(ab′)2 fragments have two antigen combining sites. In contrast, Fab, Fv, ScFv and sdAb fragments are monovalent, having only one antigen combining site. Monovalent antibody fragments are particularly useful as tags, because of their small size.

A preferred binding molecule may be a sdAb. By “sdAb” we mean single domain antibody consisting of one, two or more single monomeric variable antibody domains. sdAb molecules are sometimes referred to as dAb.

In some cases, the binding molecule is a single chain antibody, or scAb. A scAb consists of covalently linked VH and VL partner domains (e.g. directly, by a peptide, or by a flexible oligopeptide) and optionally a light chain constant domain.

An antibody may be selected from 3F8, 8H9, Abagovomab, Abciximab (ReoPro), Abituzumab, Abrezekimab, Abrilumab, Actoxumab, Adalimumab (Humira), Adecatumumab, Aducanumab, Afasevikumab, Afelimomab, Alacizumab pegol, Alemtuzumab (Lemtrada), Alirocumab (Praluent), Altumomab pentetate (Hybri-ceaker), Amatuximab, Amivantamab, Anatumomab mafenatox, Andecaliximab, Anetumab ravtansine, Anifrolumab, Ansuvimab (Ebanga), Anrukinzumab (=IMA-638), Apolizumab, Aprutumab ixadotin, Arcitumomab (CEA-Scan), Ascrinvacumab, Aselizumab, Atezolizumab (Tecentriq), Atidortoxumab, Atinumab, Atoltivimab, Atoltivimab/maftivimab/odesivimab (Inmazeb), Atorolimumab, Avelumab (Bavencio), Azintuxizumab vedotin, Balstilimab, Bamlanivimab, Bapineuzumab, Basiliximab (Simulect), Bavituximab, BCD-100, Bectumomab (LymphoScan), Begelomab, Belantamab mafodotin (Blenrep), Belimumab (Benlysta), Bemarituzumab, Benralizumab (Fasenra), Berlimatoxumab, Bermekimab (Xilonix), Bersanlimab, Bertilimumab, Besilesomab (Scintimun), Bevacizumab (Avastin), Bezlotoxumab (Zinplava), Biciromab (FibriScint), Bimagrumab, Bimekizumab, Birtamimab, Bivatuzumab, Bleselumab, Blinatumomab (Blincyto), Blontuvetmab (Blontress), Blosozumab, Bococizumab, Brazikumab, Brentuximab vedotin (Adcentris), Briakinumab, Brodalumab (Siliq), Brolucizumab (Beovu), Brontictuzumab, Burosumab (Crysvita), Cabiralizumab, Camidanlumab tesirine, Camrelizumab, Canakinumab (Ilaris), Cantuzumab mertansine, Cantuzumab ravtansine, Caplacizumab (Cablivi), Casirivimab, Capromab (Prostascint), Carlumab, Carotuximab, Catumaxomab (Removab), cBR96-doxorubicin immunoconjugate, Cedelizumab, Cemiplimab (Libtayo), Cergutuzumab amunaleukin, Certolizumab pegol (Cimzia), Cetrelimab, Cetuximab (Erbitux), Cibisatamab, Cirmtuzumab, Citatuzumab bogatox, Cixutumumab, Clazakizumab, Clenoliximab, Clivatuzumab tetraxetan (hPAM4-Cide), Codrituzumab, Cofetuzumab pelidotin, Coltuximab ravtansine, Conatumumab, Concizumab, Cosfroviximab (ZMapp), Crenezumab, Crizanlizumab (Adakveo), Crotedumab, CR6261, Cusatuzumab, Dacetuzumab, Daclizumab (Zenapax), Dalotuzumab, Dapirolizumab pegol, Daratumumab (Darzalex), Dectrekumab, Demcizumab, Denintuzumab mafodotin, Denosumab (Prolia), Depatuxizumab mafodotin, Derlotuximab biotin, Detumomab, Dezamizumab, Dinutuximab (Unituxin), Dinutuximab beta (Qarziba), Diridavumab, Domagrozumab, Dorlimomab aritox, Dostarlimab, Drozitumab, DS-8201, Duligotuzumab, Dupilumab (Dupixent), Durvalumab (Imfinzi), Dusigitumab, Duvortuxizumab, Ecromeximab, Eculizumab (Soliris), Edobacomab, Edrecolomab (Panorex), Efalizumab (Raptiva), Efungumab (Mycograb), Eldelumab, Elezanumab, Elgemtumab, Elotuzumab (Empliciti), Elsilimomab, Emactuzumab, Emapalumab (Gamifant), Emibetuzumab, Emicizumab (Hemlibra), Enapotamab vedotin, Enavatuzumab, Enfortumab vedotin (Padcev), Enlimomab pegol, Enoblituzumab, Enokizumab, Enoticumab, Ensituximab, Epcoritamab, Epitumomab cituxetan, Epratuzumab, Eptinezumab (Vyepti), Erenumab (Aimovig), Erlizumab, Ertumaxomab (Rexomun), Etaracizumab (Abegrin), Etesevimab, Etigilimab, Etrolizumab, Evinacumab (Evkeeza), Evolocumab (Repatha), Exbivirumab, Fanolesomab (NeutroSpec), Faralimomab, Faricimab, Farletuzumab, Fasinumab, FBTA05 (Lymphomun), Felvizumab, Fezakinumab, Fibatuzumab, Ficlatuzumab, Figitumumab, Firivumab, Flanvotumab, Fletikumab, Flotetuzumab, Fontolizumab (HuZAF), Foralumab, Foravirumab, Fremanezumab (Ajovy), Fresolimumab, Frovocimab, Frunevetmab, Fulranumab, Futuximab, Galcanezumab (Emgality), Galiximab, Gancotamab, Ganitumab, Gantenerumab, Gatipotuzumab, Gavilimomab, Gedivumab, Gemtuzumab ozogamicin (Mylotarg), Gevokizumab, Gilvetmab, Gimsilumab, Girentuximab (Rencarex), Glembatumumab vedotin, Golimumab (Simponi), Gomiliximab, Gosuranemab, Guselkumab (Tremfya), Ianalumab, Ibalizumab (Trogarzo), IBI308, Ibritumomab tiuxetan (Zevalin), Icrucumab, Idarucizumab (Praxbind), Ifabotuzumab, Igovomab (Indimacis-125), Iladatuzumab vedotin, IMAB362, Imalumab, Imaprelimab, Imciromab (Myoscint), Imdevimab, Imgatuzumab, Inclacumab, Indatuximab ravtansine, Indusatumab vedotin, Inebilizumab (Uplizna), Infliximab (Remicade), Intetumumab, Inolimomab, Inotuzumab ozogamicin (Besponsa), Ipilimumab (Yervoy), Iomab-B, Iratumumab, Isatuximab (Sarclisa), Iscalimab, Istiratumab, Itolizumab (Alzumab), Ixekizumab (Taltz), Keliximab, Labetuzumab (CEA-Cide), Lacnotuzumab, Ladiratuzumab vedotin, Lampalizumab, Lanadelumab (Takhzyro), Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab (Cytopoint), Lorvotuzumab mertansine, Lucatumumab, Lulizumab pegol, Lumiliximab, Lumretuzumab, Lupartumab, Lupartumab amadotin, Lutikizumab, Maftivimab, Mapatumumab, Margetuximab (Margenza), Marstacimab, Maslimomab, Mavrilimumab, Matuzumab, Mepolizumab (Bosatria), Metelimumab, Milatuzumab, Minretumomab, Mirikizumab, Mirvetuximab soravtansine, Mitumomab, Modotuximab, Mogamulizumab (Poteligeo), Monalizumab, Morolimumab, Mosunetuzumab, Motavizumab (Numax), Moxetumomab pasudotox (Lumoxiti), Muromonab-CD3 (Orthoclone OKT3), Nacolomab tafenatox, Namilumab, Naptumomab estafenatox, Naratuximab emtansine, Narnatumab, Narsoplimab, Natalizumab (Tysabri), Navicixizumab, Navivumab, Naxitamab (Danyelza), Nebacumab, Necitumumab (Portrazza), Nemolizumab, NEOD001, Nerelimomab, Nesvacumab, Netakimab (Efleira), Nimotuzumab (BioMab-EGFR, Theracim, Theraloc), Nirsevimab, Nivolumab (Opdivo), Nofetumomab merpentan (Verluma), Obiltoxaximab (Anthim), Obinutuzumab (Gazyva), Ocaratuzumab, Ocrelizumab (Ocrevus), Odesivimab, Odulimomab, Ofatumumab (Arzerra, Kesimpta), Olaratumab (Lartruvo), Oleclumab, Olendalizumab, Olokizumab, Omalizumab (Xolair), Omburtamab, OMS721, Onartuzumab, Ontuxizumab, Onvatilimab, Opicinumab, Oportuzumab monatox (Vicinium), Oregovomab (OvaRex), Orticumab, Otelixizumab, Otilimab, Otlertuzumab, Oxelumab, Ozanezumab, Ozoralizumab, Pagibaximab, Palivizumab (Synagis, Abbosynagis), Pamrevlumab, Panitumumab (Vectibix), Pankomab, Panobacumab, Parsatuzumab, Pascolizumab, Pasotuxizumab, Pateclizumab, Patritumab, PDR001, Pembrolizumab (Keytruda), Pemtumomab (Theragyn), Perakizumab, Pertuzumab (Perjeta), Pexelizumab, Pidilizumab, Pinatuzumab vedotin, Pintumomab, Placulumab, Prezalumab, Plozalizumab, Pogalizumab, Polatuzumab vedotin (Polivy), Ponezumab, Porgaviximab, Prasinezumab, Prezalizumab, Priliximab, Pritoxaximab, Pritumumab, PRO 140, Quilizumab, Racotumomab (Vaxira), Radretumab, Rafivirumab, Ralpancizumab, Ramucirumab (Cyramza), Ranevetmab, Ranibizumab (Lucentis), Raxibacumab, Ravagalimab, Ravulizumab (Ultomiris), Refanezumab, Regavirumab, Regdanvimab, REGN-EB3, Relatlimab, Remtolumab, Reslizumab (Cinqair), Retifanlimab, Rilotumumab, Rinucumab, Risankizumab (Skyrizi), Rituximab (MabThera, Rituxan), Rivabazumab pegol, Robatumumab, Rmab (RabiShield), Roledumab, Romilkimab, Romosozumab (Evenity), Rontalizumab, Rosmantuzumab, Rovalpituzumab tesirine, Rovelizumab (LeukArrest), Rozanolixizumab, Ruplizumab (Antova), SA237, Sacituzumab govitecan (Trodelvy), Samalizumab, Samrotamab vedotin, Sarilumab (Kevzara), Satralizumab (Enspryng), Satumomab pendetide, Secukinumab (Cosentyx), Selicrelumab, Seribantumab, Setoxaximab, Setrusumab, Sevirumab, Sibrotuzumab, SGN-CD19A, SHP647, Sifalimumab, Siltuximab (Sylvant), Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab (LeukoScan), Suptavumab, Sutimlimab, Suvizumab, Suvratoxumab, Tabalumab, Tacatuzumab tetraxetan (AFP-Cide), Tadocizumab, Tafasitamab (Monjuvi), Talacotuzumab, Talizumab, Talquetamab, Tamtuvetmab (Tactress), Tanezumab, Taplitumomab paptox, Tarextumab, Tavolimab, Teclistamab, Tefibazumab (Aurexis), Telimomab aritox, Telisotuzumab, Telisotuzumab vedotin, Tenatumomab, Teneliximab, Teplizumab, Tepoditamab, Teprotumumab (Tepezza), Tesidolumab, Tetulomab, Tezepelumab, TGN1412, Tibulizumab, Tildrakizumab (llumya), Tigatuzumab, Timigutuzumab, Timolumab, Tiragolumab, Tiragotumab, Tislelizumab, Tisotumab vedotin, TNX-650, Tocilizumab (Actemra, RoActemra), Tomuzotuximab, Toralizumab, Toripalimab (Tuoyi), Tosatoxumab, Tositumomab (Bexxar), Tovetumab, Tralokinumab, Trastuzumab (Herceptin), [fam]-trastuzumab deruxtecan (Enhertu), Trastuzumab duocarmazine (Kadcyla), Trastuzumab emtansine (Kadcyla), TRBS07 (Ektomab), Tregalizumab, Tremelimumab, Trevogrumab, Tucotuzumab celmoleukin, Tuvirumab, Ublituximab, Ulocuplumab, Urelumab, Urtoxazumab, Ustekinumab (Stelara), Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab (Entyvio), Veltuzumab, Vepalimomab, Vesencumab, Visilizumab (Nuvion), Vobarilizumab, Volociximab, Vonlerolizumab, Vopratelimab, Vorsetuzumab mafodotin, Votumumab (HumaSPECT), Vunakizumab, Xentuzumab, XMAB-5574, Zalutumumab, Zanolimumab, Zatuximab, Zenocutuzumab, Ziralimumab, Zolbetuximab (=IMAB362, Claudiximab), and Zolimomab aritox. An antigen-binding molecule may be a derivative of any of the abovementioned antibodies.

Where the antibody is encoded by the cargo, the nucleotide sequence encoding the antibody may be modified. The amino acid sequence of the antibody may be modified. For example, the nucleotide and/or amino acid sequence may comprise modifications which enable in vivo expression or improve in vivo expression of the antibody.

### Amount and Size Loaded

Extracellular vesicle(s) according to the present disclosure may comprise at least two nucleic acids, wherein each of the nucleic acids is different. The extracellular vesicle(s) may contain more than two nucleic acids. In such cases, at least two of the nucleic acids is different, but there may be additional nucleic acids that are identical to one of the at least two different nucleic acids. What is important in such cases is that the cargo is not completely homologous, or not 100% identical, but instead contains at least one molecule that is different or not identical, to other molecules of the cargo.

The number of the nucleic acid(s) per vesicle may be an average number, preferably mean average, across a population of EVs, e.g. as present in a composition. The number of copies of nucleic acid per vesicle may be determined by dividing the total number of copies of the loaded nucleic acid cargo by the total number of EVs. In other words, Copies per EV=Number of loaded copies of nucleic acid/Total number of EV particles. The number of copies of nucleic acid may be determined by qPCR. The number of EVs may be determined by nanoparticle tracking analysis (NTA, e.g. as described in Wang et al., ARMMs as a versatile platform for intracellular delivery of macromolecules. Nature Communications 2018 9-960). Nanoparticle tracking analysis (NTA) is a method for visualizing and analyzing particles in liquids. The technique is used in conjunction with an ultramicroscope and a laser illumination unit that together allow small particles in liquid suspension to be visualized moving under Brownian motion. The light scattered by the particles is captured using a CCD or EMCCD camera over multiple frames. Computer software is then used to track the motion of each particle from frame to frame.

As used herein and unless indicated otherwise, the term “average” refers to the mathematical mean. This may refer to the total amount of nucleic acid determined in a sample, divided by the total number of vesicles in that sample.

Compositions described herein comprise at least one extracellular vesicle that is loaded with at least 2 different nucleic acids, or at least 3 different nucleic acids. These extracellular vesicles may comprise at least 1, at least 2, at least 3, at least 3.5, at least 4, at least 5 or more copies of the nucleic acid per vesicle. The extracellular vesicle(s) may comprise (e.g. be loaded with) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more copies of each of the different nucleic acids per vesicle. The extracellular vesicle(s) may comprise (e.g. be loaded with) about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nucleic acids per vesicle, wherein at least one of the nucleic acids is different, or not-identical to, other nucleic acids of the cargo. In some cases, the extracellular vesicle(s) may comprise approximately equal numbers of each nucleic acid (i.e. about 50% of each nucleic acid, or about 48%, or about 46%, or about 44%, or about 42%, or about 40%, or about 38%, or about 36%, or about 34%, or about 32% or about 30% of each nucleic acid). In some cases, the extracellular vesicle(s) may comprise different numbers of each nucleic acid (i.e. about 2% of the nucleic acids in the extracellular vesicle(s) are different to the other nucleic acids of the cargo, or about 4%, or about 6%, or about 8%, or about 10%, or about 12%, or about 14%, or about 16%, or about 18%, or about 20%, or about 22%, or about 24%, or about 25%, or about 26%, or about 28% or about 30%).

Although it may be desirable for the cargo to be loaded into substantially all of the extracellular vesicles in a composition, compositions disclosed herein may comprise extracellular vesicles in which one of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% of the extracellular vesicles comprise cargo. Preferably, at least one of 50%, 55%, 60%, 65%, 70%, 75%, 80%, 85%, 90%, 95%, 96%, 97%, 98%, 99%, or 100% of the extracellular vesicles comprise cargo. Preferably, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% of the extracellular vesicles comprise cargo, wherein the cargo includes at least two different nucleic acids. In some cases, different extracellular vesicles within the composition contain different cargo. For example, some extracellular vesicles in the composition may comprise cargo wherein the cargo of the extracellular vesicle is substantially homogenous, or contains only one biomarker. In some compositions, other extracellular vesicles in the composition comprise a cargo comprising at least two different nucleic acids. In some cases, the extracellular vesicles contain the same, or substantially the same, cargo, wherein the cargo comprises at least two different nucleic acids.

The size of a nucleic acid may be defined in terms of its length in bases (for single stranded nucleic acids) or base pairs (for double stranded nucleic acids). In this specification, where the single or double stranded nature of the nucleic acid cargo is not indicated a length given in bases (e.g. in kb (kilobases) is also a disclosure of the same length in base pairs (e.g. in kbp). As such a length of 1 kb (1000 bases) is also a disclosure of 1 kbp (1000 base pairs). The term “bases” is used interchangeably with the term “nucleotides”. The nucleic acid cargo can be single stranded or double stranded. It can be linear or circular.

Where a nucleic acid of the cargo is single stranded, each nucleic acid may have a length of one of at least 250, 500, 750, 1000, 1250, 1500, 1750, 2000, 2250, 2500, 2750, 3000, 3250, 3500, 3750, 4000, 4250, 4500, 4750, 5000, 5250, 5500, 5750, 6000, 6250, 6500, 6750, 7000, 7250, 7500, 7750, 8000, 8250, 8500, 8750, 9000, 9250, 9500, 9750, 10000, 10250, 10500, 10750 or 11000 bases. Optionally, wherein a nucleic acid of the cargo is single stranded DNA (ssDNA) it may have a maximum length of one of 4000, 4250, 4500, 4750, 5000, 5250, 5500, 5750, 6000, 6250, 6500, 6750, 7000, 7250, 7500, 7750, 8000, 8250, 8500, 8750, 9000, 9250, 9500, 9750, 10000, 10250, 10500, 10750 or 11000 bases. In preferred embodiments a single stranded nucleic acid of the cargo may have a minimum length of one of 2000, 2250, 2500, 2750, 3000, 3250, 3500, 3750, 4000, 4250, 4500, 4750, 5000 or more than 5000 bases.

Where a nucleic acid of the cargo is single stranded it may have a length of one of 250-750, 500-1000, 1000-1500, 1500-2000, 2000-2500, 2500-3000, 3000-3500, 3500-4000, 4000-4500, 4500-5000, 5000-5500, 5500-6000, 1000-2000, 2000-3000, 3000-4000, 4000-5000, 5000-6000, 6000-7000, 7000-8000, 8000-9000, 9000-10000, 10000-11000, 250-1000, 1000-3000, 1000-4000, 1000-5000, 1000-6000, 1000-7000, 1000-8000, 1000-9000, 1000-10000, 1000-11000, 2000-4000, 2000-5000, 2000-6000, 2000-7000, 2000-8000, 2000-9000, 2000-10000, 2000-11000, 3000-5000, 3000-6000, 3000-7000, 3000-8000, 3000-9000, 3000-10000, 3000-11000, 4000-6000, 4000-7000, 4000-8000, 4000-9000, 4000-10000, 4000-11000, 5000-7000, 5000-8000, 5000-9000, 5000-10000, 5000-11000, 6000-8000, 6000-9000, 6000-10000, 6000-11000, 7000-9000, 7000-10000, or 7000-11000, bases.

In some embodiments where a nucleic acid of the cargo is single stranded it may have a length of up to one of 2000, 3000, 4000, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, or 40000 bases. A single stranded nucleic acid of the cargo may have a length of one of 5000-10000, 5000-15000, 5000-20000, 5000-25000, 5000-30000, 5000-35000, 5000-40000, 10000-15000, 10000-20000, 10000-25000, 10000-30000, 10000-35000, 10000-40000, 15000-20000, 15000-25000, 15000-30000, 15000-35000, 15000-40000, 20000-25000, 20000-30000, 20000-35000, 20000-40000, 25000-30000, 25000-35000, 25000-40000, 30000-35000, 30000-40000, or 35000-40000 bases.

Where a nucleic acid of the cargo is double stranded it may have a length of one of at least 250, 500, 750, 1000, 1250, 1500, 1750, 2000, 2250, 2500, 2750, 3000, 3250, 3500, 3750, 4000, 4250, 4500, 4750, 5000, 5250, 5500, 5750, 6000, 6250, 6500, 6750, 7000, 7250, 7500, 7750, 8000, 8250, 8500, 8750, 9000, 9250, 9500, 9750, 10000, 10250, 10500, 10750 or 11000 base pairs. Optionally, where a nucleic acid of the cargo is double stranded it may have a maximum length of one of 4000, 4250, 4500, 4750, 5000, 5250, 5500, 5750, 6000, 6250, 6500, 6750, 7000, 7250, 7500, 7750, 8000, 8250, 8500, 8750, 9000, 9250, 9500, 9750, 10000, 10250, 10500, 10750 or 11000 base pairs. In preferred embodiments a double stranded nucleic acid of the cargo may have a minimum length of one of 2000, 2250, 2500, 2750, 3000, 3250, 3500, 3750, 4000, 4250, 4500, 4750, 5000 or more than 5000 base pairs.

Where a nucleic acid cargo is double stranded it may have a length of one of 250-750, 500-1000, 1000-1500, 1500-2000, 2000-2500, 2500-3000, 3000-3500, 3500-4000, 4000-4500, 4500-5000, 5000-5500, 5500-6000, 1000-2000, 2000-3000, 3000-4000, 4000-5000, 5000-6000, 6000-7000, 7000-8000, 8000-9000, 9000-10000, 10000-11000, 250-1000, 1000-3000, 1000-4000, 1000-5000, 1000-6000, 1000-7000, 1000-8000, 1000-9000, 1000-10000, 1000-11000, 2000-4000, 2000-5000, 2000-6000, 2000-7000, 2000-8000, 2000-9000, 2000-10000, 2000-11000, 3000-5000, 3000-6000, 3000-7000, 3000-8000, 3000-9000, 3000-10000, 3000-11000, 4000-6000, 4000-7000, 4000-8000, 4000-9000, 4000-10000, 4000-11000, 5000-7000, 5000-8000, 5000-9000, 5000-10000, 5000-11000, 6000-8000, 6000-9000, 6000-10000, 6000-11000, 7000-9000, 7000-10000, or 7000-11000, base pairs.

In some embodiments where a nucleic acid of the cargo is double stranded it may have a length of up to one of 2000, 3000, 4000, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, or 40000 base pairs. A double stranded nucleic acid of the cargo may have a length of one of 5000-10000, 5000-15000, 5000-20000, 5000-25000, 5000-30000, 5000-35000, 5000-40000, 10000-15000, 10000-20000, 10000-25000, 10000-30000, 10000-35000, 10000-40000, 15000-20000, 15000-25000, 15000-30000, 15000-35000, 15000-40000, 20000-25000, 20000-30000, 20000-35000, 20000-40000, 25000-30000, 25000-35000, 25000-40000, 30000-35000, 30000-40000, or 35000-40000 base pairs.

A nucleic acid of the cargo, e.g. RNA, may have a length of one of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases. A nucleic acid of the cargo may have a length of one of at least 50, 55, 60, 65, 70, 75, 80, 85, 90, 95 or 100 bases. A nucleic acid of the cargo may have a length of one of at least 100, 110, 120, 130, 140, 150, 160, 170, 180, 190, 200, 210, 220, 230, 240 or 250 bases.

Where a nucleic acid of the cargo is single stranded, e.g. single stranded RNA, it may have a length of one of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases; at least 50, 55, 60, 65, 70, 75, 80, 85, 90, 95 or 100 bases; or at least 100, 110, 120, 130, 140, 150, 160, 170, 180, 190, 200, 210, 220, 230, 240 or 250 bases.

Where a nucleic acid of the cargo is double stranded, e.g. double stranded RNA such as siRNA, it may have a length of one of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs. A nucleic acid of the cargo may have a length of one of at least 50, 55, 60, 65, 70, 75, 80, 85, 90, 95 or 100 base pairs. A nucleic acid of the cargo may have a length of one of at least 100, 110, 120, 130, 140, 150, 160, 170, 180, 190, 200, 210, 220, 230, 240 or 250 base pairs.

Each nucleic acid of the cargo may be between about 0.5 kb and about 4 kb, between about 0.5 kb and about 3 kb, between about 0.5 kb and about 2.5 kb, between about 1 kb and about 3 kb, between about 1.5 kb and about 2.5 kb, or about 2 kb. Each nucleic acid cargo may be at least 0.5 kb, at least 1.0 kb, at least 1.5 kb, at least 2.0 kb, at least 2.5 kb, at least 3 kb, at least 4 kb, at least 5 kb, at least 6 kb, at least 7 kb, at least 8 kb, at least 9 kb, at least 10 kb, at least 11 kb, at least 12 kb, at least 13 kb, at least 14 kb, at least 15 kb, at least 16 kb, at least 17 kb, at least 18 kb, at least 19 kb, at least 20 kb, at least 21 kb, at least 22 kb, at least 23 kb, at least 24 kb, at least 25 kb, at least 26 kb, at least 27 kb, at least 28 kb, at least 29 kb, at least 30 kb, at least 31 kb, at least 32 kb, at least 33 kb, at least 34 kb, at least 35 kb, at least 36 kb, at least 37 kb, at least 38 kb, at least 39 kb, at least 40 kb, at least 41 kb, at least 42 kb, at least 43 kb, at least 44 kb, at least 45 kb, at least 46 kb, at least 47 kb, at least 48 kb, at least 49 kb, at least 50 kb or more. In some preferred embodiments each nucleic acid of the cargo is at least 2 kb.

In some cases, the total nucleic acid cargo may be between about 0.5 kb and about 4 kb, between about 0.5 kb and about 3 kb, between about 0.5 kb and about 2.5 kb, between about 1 kb and about 3 kb, between about 1.5 kb and about 2.5 kb, or about 2 kb. Each nucleic acid cargo may be at least 0.5 kb, at least 1.0 kb, at least 1.5 kb, at least 2.0 kb, at least 2.5 kb, at least 3 kb, at least 4 kb, at least 5 kb, at least 6 kb, at least 7 kb, at least 8 kb, at least 9 kb, at least 10 kb, at least 11 kb, at least 12 kb or more. In other words, that the cargo comprises multiple nucleic acids, and the combined length of these nucleic acids in each vesicle is, on average, between about 0.5 kb and about 4 kb, between about 0.5 kb and about 3 kb, between about 0.5 kb and about 2.5 kb, between about 1 kb and about 3 kb, between about 1.5 kb and about 2.5 kb, or about 2 kb. Each nucleic acid cargo may be at least 0.5 kb, at least 1.0 kb, at least 1.5 kb, at least 2.0 kb, at least 2.5 kb, at least 3 kb, at least 4 kb, at least 5 kb, at least 6 kb, at least 7 kb, at least 8 kb, at least 9 kb, at least 10 kb, at least 11 kb, at least 12 kb, at least 13 kb, at least 14 kb, at least 15 kb, at least 16 kb, at least 17 kb, at least 18 kb, at least 19 kb, at least 20 kb, at least 21 kb, at least 22 kb, at least 23 kb, at least 24 kb, at least 25 kb, at least 26 kb, at least 27 kb, at least 28 kb, at least 29 kb, at least 30 kb, at least 31 kb, at least 32 kb, at least 33 kb, at least 34 kb, at least 35 kb, at least 36 kb, at least 37 kb, at least 38 kb, at least 39 kb, at least 40 kb, at least 41 kb, at least 42 kb, at least 43 kb, at least 44 kb, at least 45 kb, at least 46 kb, at least 47 kb, at least 48 kb, at least 49 kb, at least 50 kb or more.

The cargo may comprise at least 2 non-identical nucleic acids. The combined length of a first nucleic acid of the cargo and a further non-identical nucleic acid of the cargo may be 0.5 kb, at least 1.0 kb, at least 1.5 kb, at least 2.0 kb, at least 2.5 kb, at least 3 kb, at least 4 kb, at least 5 kb, at least 6 kb, at least 7 kb, at least 8 kb, at least 9 kb, at least 10 kb, at least 11 kb, at least 12 kb, at least 13 kb, at least 14 kb, at least 15 kb, at least 16 kb, at least 17 kb, at least 18 kb, at least 19 kb, at least 20 kb, at least 21 kb, at least 22 kb, at least 23 kb, at least 24 kb, at least 25 kb, at least 26 kb, at least 27 kb, at least 28 kb, at least 29 kb, at least 30 kb, at least 31 kb, at least 32 kb, at least 33 kb, at least 34 kb, at least 35 kb, at least 36 kb, at least 37 kb, at least 38 kb, at least 39 kb, at least 40 kb, at least 41 kb, at least 42 kb, at least 43 kb, at least 44 kb, at least 45 kb, at least 46 kb, at least 47 kb, at least 48 kb, at least 49 kb, at least 50 kb or more. The combined length of a first nucleic acid of the cargo and a further non-identical nucleic acid of the cargo may be between about 0.5 kb and about 4 kb, between about 0.5 kb and about 3 kb, between about 0.5 kb and about 2.5 kb, between about 1 kb and about 3 kb, between about 1.5 kb and about 2.5 kb, or about 2 kb.

We have defined the size of a nucleic acid in terms of its length in bases or base pairs. However, the size of a nucleic acid may also be influenced by the structure of the nucleic acid. For example, a supercoiled DNA plasmid would have a more compact structure than a linear DNA having the same number of base pairs.

In some cases, the nucleic acids of the cargo are homogeneous (i.e. each nucleic acid in a composition of EVs is similar or substantially identical). In some cases, the nucleic acids of the cargo are heterogeneous (i.e. the nucleic acids in a composition of EVs are not similar or substantially identical to each other).

Suitable small molecules include cytotoxic reagents and kinase inhibitors. The small molecule may comprise a fluorescent probe and/or a metal. For example, the cargo may comprise a superparamagnetic particle such as an iron oxide particle. The cargo may be an ultra-small superparamagnetic iron oxide particle such as an iron oxide nanoparticle.

In some cases, the cargo is a detectable moiety such as a fluorescent dextran. The cargo may be radioactively labelled.

### Method of Loading Extracellular Vesicles

In this specification, loading of an extracellular vesicle with a cargo refers to associating the extracellular vesicle and cargo in stable or semi-stable form such that the extracellular vesicle is useful as a carrier of the cargo, e.g. allowing its delivery to cells. Cargo molecules may be loaded in at least two ways. One is for the cargo to be present in the lumen of the extracellular vesicle (lumenal loading). Another is for the cargo to be attached to, adhered to, inserted through, or complexed with the external surface, e.g. membrane, of the extracellular vesicle (external surface loading). Cargo molecules loaded onto the external surface of the extracellular vesicle may usually be removed by contacting the vesicle with a nuclease, e.g. a DNase or RNase.

Methods are disclosed herein for loading an extracellular vesicle with two or more non-identical nucleic acids. The method may comprise a step of contacting an extracellular vesicle with a mixture comprising a plurality of non-identical nucleic acids to be loaded. The method may involve a step of contacting an extracellular vesicle with a first nucleic acid to be loaded, followed by a step of contacting the extracellular vesicle with a second or further nucleic acid to be loaded which is non-identical to the first nucleic acid.

### Transfection Reagents

In some cases, where the nucleic acid to be loaded is a nucleic acid, extracellular vesicle(s), nucleic acid and transfection reagent are brought together under suitable conditions and for sufficient time to allow loading to occur.

Loading methods may include contacting a nucleic acid to be loaded with a transfection reagent. Suitable transfection reagents include cationic reagents such as cationic lipid reagents. Several transfection reagents are known in the art, including Lipofectamine™ 3000™ (ThermoFisher), Turbofect™ (ThermoFisher), Lipofectamine™ MessengerMAX™ (ThermoFisher), Exofect™ (System Biosciences), and Linear Polyethylenimine Hydrochlorides, e.g. having an average molecular weight of 25,000 Da or 40,000 Da, such as PEIMax™ (Polysciences, Inc.) and jetPEI® (Polyplus transfection).

Some methods disclosed herein involve a step of preparing the nucleic acid to be loaded. The nucleic acid to be loaded may comprise a plurality of non-identical nucleic acids. In the preparing step, the nucleic acid that is to be loaded into the extracellular vesicle is contacted with the transfection reagent under conditions suitable for the formation of a complex between the transfection reagent and the nucleic acid. The nucleic acid and the transfection reagent are contacted for sufficient time for complex formation to occur. Preferably, the nucleic acid and transfection reagent form a complex, such as a DNA:PEIMax complex. Preparation of the nucleic acid for loading may comprise further steps, such as concentration or dilution of the nucleic acid, or the addition of buffers or other reagents or media, such as Opti-MEM reduced serum media (Gibco). The nucleic acid and the transfection reagent may be contacted for at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 16 minutes, at least 17 minutes, at least 18 minutes, at least 19 minutes, at least 20 minutes or more than 20 minutes. Preparation of the nucleic acid for loading may comprise the further step of combining a nucleic acid:transfection reagent complex with a further nucleic acid:transfection reagent complex wherein the nucleic acids are non-identical.

Methods disclosed herein may involve a step of loading the extracellular vesicles with the nucleic acid:transfection reagent complexes. Prepared nucleic acid:transfection reagent complexes are contacted with the extracellular vesicle that is to be loaded. In preferred methods, the extracellular vesicles are added to prepared nucleic acid:transfection reagent complexes. In other words, contacting with the extracellular vesicle is performed subsequently to the contacting of the nucleic acid to be loaded with the transfection reagent. Normally, the nucleic acid:transfection reagent complexes are contacted with a composition comprising a plurality of extracellular vesicles. The nucleic acid:transfection reagent complexes and extracellular vesicle may be incubated for sufficient time and under appropriate conditions to allow the extracellular vesicle to be loaded with one or more of the nucleic acid:transfection reagent complexes. The complexes may be internalised into the extracellular vesicle, or otherwise loaded onto the extracellular vesicle, such as onto the surface of the extracellular vesicle. Preferably, the complexes are internalised into the extracellular vesicle.

Following the loading step, the extracellular vesicles may be isolated, washed and/or concentrated. In preferred methods, a washing step follows the loading step. Following the loading step, the mixture may be washed with PBS. Preferably, washing comprises centrifuging the mixture to pellet the extracellular vesicles, resuspending the pellet in an appropriate buffer (such as PBS). The washing step may be repeated 1, 2, 3, 4, 5, 6 or more times.

The step of loading the extracellular vesicles with nucleic acid:transfection reagent complexes may be repeated. In other words, following a step of loading extracellular vesicles with nucleic acid:transfection reagent complexes, the extracellular vesicles may be optionally washed and contacted with further nucleic acid: transfection reagent complexes. The further nucleic acid:transfection reagent complexes may comprise a nucleic acid which is non-identical to a nucleic acid loaded in the previous loading step. In such methods, the extracellular vesicles to be loaded with nucleic acid:transfection reagent complexes may be loaded extracellular vesicles, and thus may already contain nucleic acid cargo. Alternatively, the extracellular vesicles may have been subject to a loading step, but have not been loaded with cargo, or have been loaded with a low level of cargo. Where a second or further loading step is required, the extracellular vesicles may be incubated with the further nucleic acid:transfection reagent complexes under the same or different conditions, and for the same or different time, as used in the preceding loading step. Following the second or further loading step, a further washing step may be used.

Preferably, the method involves incubating extracellular vesicles with nucleic acid:transfection reagent complexes, and does not involve incubating cells with nucleic acid:transfection reagent complexes and subsequently inducing the formation of extracellular vesicles from such cells.

In some cases, methods suitable for loading cargo into the extracellular vesicles may require a temporary or semi-permanent increase in the permeability of the membrane of the extracellular vesicle. Suitable methods are described in PCT/SG2018/050596 and include, for example, electroporation, sonication, ultrasound, lipofection or hypotonic dialysis. In methods disclosed herein, extracellular vesicles are contacted with a cargo to form a mixture, and the mixture is treated to increase the permeability of the membrane of the extracellular vesicles. The mixture may be chilled prior to treatment. It may further involve one or more buffers, such as PBS.

The step of loading the extracellular vesicles may be repeated. In other words, following a first treatment step, the extracellular vesicles may optionally be washed and contacted with a further cargo to form a mixture. The mixture is then treated to increase the permeability of the membrane of the extracellular vesicles. The further cargo may comprise a plurality of non-identical nucleic acids. Alternatively, the further cargo may comprise a population of nucleic acids which are non-identical to the nucleic acids of a prior loading step.

Cargo may be loaded into the extracellular vesicles by electroporation. Electroporation, or electropermeabilization, is a microbiology technique in which an electrical field is applied to cells in order to increase the permeability of the cell membrane, allowing chemicals, drugs, or DNA to be introduced into the cell. In other words, the extracellular vesicles may be induced or forced to encapsulate the cargo by electroporation. Electroporation works by passing thousands of volts across a distance of one to two millimeters of suspended cells in an electroporation cuvette (1.0-1.5 KV, 250-750V/cm). Generally, electroporation is a multi-step process, with several distinct phase. First, a short electrical pulse is applied. Typical parameters would be 300-400 mV for <1 ms across the membrane. Upon application of this potential the membrane charges like a capacitor through the migration of ions from the surrounding solution. Once the critical field is achieved there is a rapid localized rearrangement in lipid morphology. The resulting structure is believed to be a “pre-pore” since it is not electrically conductive but leads rapidly to the creation of a conductive pore. Evidence for the existence of such pre-pores comes mostly from the “flickering” of pores, which suggests a transition between conductive and insulating states. It has been suggested that these pre-pores are small (˜3 Å) hydrophobic defects. If this theory is correct, then the transition to a conductive state could be explained by a rearrangement at the pore edge, in which the lipid heads fold over to create a hydrophilic interface. Finally, these conductive pores can either heal, resealing the bilayer or expand, eventually rupturing it. The resultant fate depends on whether the critical defect size was exceeded which in turn depends on the applied field, local mechanical stress and bilayer edge energy. The success of in vivo electroporation depends greatly on voltage, repetition, pulses, and duration. Methods disclosed herein may involve subjecting extracellular vesicles to electroporation at between about 25 and 300 V, or between about 50 and 250V.

Alternatively, cargo may be loaded into the extracellular vesicles by sonication. Sonication is the act of applying sound energy to agitate particles in a sample, for various purposes such as the extraction of multiple compounds from plants, microalgae and seaweeds. Ultrasonic frequencies (>20 kHz) are usually used, leading to the process also being known as ultrasonication or ultra-sonication. Sonication may be applied using an ultrasonic bath or an ultrasonic probe, colloquially known as a sonicator.

In another method, cargo is loaded with ultrasound. Ultrasound has been shown to disrupt cell membranes, and thereby load cells with molecules. Sound waves with frequencies from 20 kHz up to several gigahertz may be applied to the extracellular vesicles.

In yet another method, cargo may be loaded into extracellular vesicles by lipofection. Lipofection (or liposome transfection) is a technique used to inject genetic material into a cell by means of liposomes, which are vesicles that can easily merge with the cell membrane since they are both made of a phospholipid bilayer.

### Mixture to be Loaded

Methods described herein may involve the preparation or provision of a mixture of nucleic acids to be loaded. The mixture is a mixture of nucleic acids. The mixture may additionally comprise transfection reagent. The mixture may comprise nucleic acid:transfection reagent complexes. The mixture may contain nucleic acids present in particular ratios. By providing the nucleic acids in the mixture in particular ratios, the likelihood that an extracellular vesicle will be loaded with nucleic acids, wherein at least one of the nucleic acids is different to the other nucleic acids is increased.

In some cases, the mixture is prepared from two or more sub-mixtures, where each sub-mixtures comprises one of the nucleic acids to be loaded and a transfection reagent. The sub-mixtures are then combined to form the mixture. For example, two or more sub-mixtures, each comprising a nucleic acid to be loaded and a transfection reagent may be combined to form the mixture. In these examples, each of the sub-mixtures comprises nucleic acid:transfection reagent complexes.

In the mixture, the first nucleic acid to be loaded and the further biomolecule may be present in equal molar amounts, i.e. at an equimolar ratio. The first nucleic acid to be loaded and the further nucleic acid to be loaded may be present in different molar amounts, i.e. not at an equimolar ratio. The ratio may refer to the amount of a first nucleic acid in relation to a further nucleic acid present in a mixture, allowing for simultaneous contact with extracellular vesicles. Alternatively, the ratio may refer to the amount of a first nucleic acid in relation to a further nucleic acid, wherein the first nucleic acid and further nucleic acid are to be contacted with extracellular vesicles in separate steps.

The first nucleic acid to be loaded and the further nucleic acid to be loaded may be present at a ratio of about 100:1, 75:1, 50:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:50, 1:75, 1:100, 1:150, 1:200, 1:250, 1:300, 1:400, 1:500. Optionally, wherein the first nucleic acid to be loaded and the further nucleic acid are present at a ratio of 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25. Preferably, the ratio is about 1:1. The ratio refers to the ratio of molar amounts of each of the first and further nucleic acid.

The first nucleic acid to be loaded and the further nucleic acid to be loaded may be present at a ratio of between 100:1-1:100, 75:1-1:75, 50:1-1:50, 25:1-1:25, 20:1-1:20, 15:1-1:15, 10:1-1:10, 9:1-1:9, 8:1-1:8, 7:1-1:7, 6:1-1:6, 5:1-1:5, 4:1-1:4, 3:1-1:3, 2:1-1:2, or about 1:1. Preferably, the first nucleic acid to be loaded and the further nucleic acid to be loaded are present in a ratio of between 1:3-3:1, 1:2-2:1 or about 1:1. The ratio refers to the ratio of molar amounts of each of the first and further nucleic acids.

Where three different nucleic acids are to be loaded into the extracellular vesicle, the first, second and third biomarkers may be present in the mixture in approximately equimolar ratios, that is, to say 1:1:1. The first, second and third nucleic acids to be loaded may be present in a ratio of about 1:1:2, 1:1:3, 1:1:4, 1:1:5, 1:1:6, 1:1:7, 1:1:8, 1:1:9, 1:1:10, 1:2:1, 1:3:1, 1:4:1, 1:5:1, 1:6:1, 1:7:1, 1:8:1, 1:9:1, 1:10:1, 2:1:1, 3:1:1, 4:1:1, 5:1:1, 6:1:1, 7:1:1, 8:1:1, 9:1:1, 10:1:1, 1:2:2, 1:3:3. 1:4:4, 1:5:5, 1:6:6, 1:7:7. 1:8:8: 1:9:9, 1:10:10, 1:2:3, 1:2:4, 1:3:6, 1:4:8, 1:5:10, 2:4:6, 2:8:4 or other ratio.

In some embodiments the nucleic acids to be loaded are nucleic acids. The first nucleic to be loaded and the further nucleic acid to be loaded may be present in equal molar amounts, i.e. at an equimolar ratio. The first nucleic acid to be loaded and the further nucleic acid to be loaded may be present in different molar amounts, i.e. not at an equimolar ratio.

The relative lengths of the nucleic acids to be loaded may influence the ratio. For example, larger nucleic acids may have a lower efficiency of loading. Therefore, where a first nucleic acid to be loaded is significantly larger than a further nucleic acid to be loaded, the larger nucleic acid may be present in higher amounts. The relative structures of the nucleic acids to be loaded may influence the ratio. For example, a DNA plasmid may have a more compact structure than a linear DNA of the same length. Nucleic acids having a more compact structure may have a higher loading efficiency. Therefore, a nucleic acid having a more compact structure may be present in lower amounts. Similarly, the ratio may be adjusted where one of the nucleic acids to be loaded is single stranded and a further nucleic acid to be loaded is double stranded. In such cases, it may be appropriate to adjust the ratio to compensate for the number of strands. For example the proportion of the single stranded component may be increased as compared to the proportion of the double stranded component. In some cases, the proportion of the single stranded component may be doubled as compared to the proportion of the double stranded component. Thus, the ratio may be adjusted from 1:1 to 2:1 where the first nucleic acid is a single stranded nucleic acid, and the further nucleic acid is a double stranded nucleic acid.

The nucleic acids may be present at a ratio of 400:1, 300:1, 250:1, 200:1, 150:1, 100:1, 75:1, 50:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:50, 1:75, 1:100, 1:150, 1:200, 1:250, 1:300, 1:400, 1:500. Optionally, wherein the first nucleic acid to be loaded and the further nucleic acid are present at a ratio of 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25. In some cases, the first nucleic acid to be loaded and the further nucleic acid to be loaded may be present at a ratio of between 100:1-1:100, 75:1-1:75, 50:1-1:50, 25:1-1:25, 20:1-1:20, 15:1-1:15, 10:1-1:10, 9:1-1:9, 8:1-1:8, 7:1-1:7, 6:1-1:6, 5:1-1:5, 4:1-1:4, 3:1-1:3, 2:1-1:2, or about 1:1. Preferably, the first nucleic acid to be loaded and the further nucleic acid to be loaded are present in a ratio of between 1:3-3:1, 1:2-2:1 or about 1:1. The ratio refers to the ratio of molar amounts of each of the first and further nucleic acids.

Where three different nucleic acids are to be loaded, these may be present in an approximately equimolar ratio (i.e. 1:1:1), or about 1:1:2, 1:1:3, 1:1:4, 1:1:5, 1:1:6, 1:1:7, 1:1:8, 1:1:9, 1:1:10, 1:2:1, 1:3:1, 1:4:1, 1:5:1, 1:6:1, 1:7:1, 1:8:1, 1:9:1, 1:10:1, 2:1:1, 3:1:1, 4:1:1, 5:1:1, 6:1:1, 7:1:1, 8:1:1, 9:1:1, 10:1:1, 1:2:2, 1:3:3. 1:4:4, 1:5:5, 1:6:6, 1:7:7. 1:8:8: 1:9:9, 1:10:10, 1:2:3, 1:2:4, 1:3:6, 1:4:8, 1:5:10, 2:4:6, 2:8:4 or other ratio.

In some embodiments where the nucleic acids to be loaded are of similar size the nucleic acids may be present at an equimolar ratio. In some embodiments where the nucleic acids to be loaded are plasmids the plasmids may be present at an equimolar ratio.

Extracellular vesicles may be loaded by a combination of lumenal and external surface loading, and such extracellular vesicles may effectively deliver cargo nucleic acids to target cells.

Optionally, in some embodiments, reference to loading may be only to lumenal loading. Optionally, in some other embodiments, reference to loading may be only to external surface loading.

In some embodiments, loading of cargo into extracellular vesicles described herein does not comprise viral delivery methods, e.g. the loading methods do not involve a viral vector such as an adenoviral, adeno-associated, lentiviral, or retroviral vector.

The method may involve a step of removing nucleic acid cargo not contained within the lumen of the extracellular vesicle. Such a step may comprise contacting the loaded extracellular vesicle with DNAse. The loaded extracellular vesicle may be contacted with heparin prior to contact with DNAse, in order to dissociate nucleic acid or nucleic acid:transfection reagent complexes.

### Compositions

Disclosed herein are compositions comprising extracellular vesicles.

The compositions may comprise between 10⁶ to 10¹⁵ particles per ml. The compositions may comprise at least 10⁵ particles per ml, at least 10⁶ particles per ml, at least at least 10⁷ particles per ml, at least 10⁸ particles per ml, at least 10⁹ particles per ml, at least 10¹⁰ particles per ml, at least 10¹¹ particles per ml, at least 10¹² particles per ml, at least 10¹³ particles per ml, at least 10¹⁴ particles per ml, of at least 10¹⁵ particles per ml.

The composition may comprise extracellular vesicles have substantially homologous dimensions. For example, the extracellular vesicles may have diameters ranging from 100-500 nm. In some cases, a composition of microvesicles comprises microvesicles with diameters ranging from 50-1000 nm, from 101-1000 nm, from 101-750 nm, from 101-500 nm, or from 100-300 nm, or from 101-300 nm. Preferably, the diameters are from 100-300 nm. In some compositions, the mean diameter of the microvesicles is 100-300 nm, preferably 150-250 nm, preferably about 200 nm.

The extracellular vesicles contain a cargo. Although it is desirable in such compositions for the cargo to be encapsulated into substantially all of the extracellular vesicles in a composition, compositions disclosed herein may comprise extracellular vesicles in which at least 30%, at least 35%, at least 40%, at least 45%, at least 50%, at least 55%, at least 60%, at least 65%, at least 70%, at least 75%, at least 80%, at least 85%, at least 90%, at least 95%, or at least 97% of the extracellular vesicles contain the cargo. Preferably, at least 85%, at least 90%, at least 95%, or at least 97% of the extracellular vesicles contain the cargo. In some cases, different extracellular vesicles within the composition contain different cargo. In some cases, the extracellular vesicles contain the same, or substantially the same, cargo.

The composition may be a pharmaceutical composition. The composition may comprise one or more extracellular vesicle, and optionally a pharmaceutically acceptable carrier. Pharmaceutical compositions may be formulated for administration by a particular route of administration. For example, the pharmaceutical composition may be formulated for intravenous, intratumoral, intraperitoneal, intradermal, subcutaneous, intranasal or other administration route.

Compositions may comprise a buffer solution. Compositions may comprise a preservative compound. Compositions may comprise a pharmaceutically acceptable carrier.

### Delivering Two or More Nucleic Acids

The EVs described herein may be used in methods of delivering two or more nucleic acids to a cell. In other words, the EVs described herein may be used as a delivery system for nucleic acids of the cargo. The cargo of an EV is delivered to the target cell. Therefore, when an EV is loaded with a cargo comprising at least two different nucleic acids said nucleic acids will be delivered simultaneously to the target cell. This ensures that the target cell receives a full complement of each of the different nucleic acids of the cargo. The EV may be loaded with a cargo as described above.

Delivering two or more nucleic acids to a target cell may be useful where a system comprises multiple components, each of which needs to be present in a single cell for the system to function, e.g. a CRISPR gene editing system. In this case two or more of a gRNA, a nucleic acid molecule encoding a nuclease, and a DNA repair template, can be delivered to the target cell simultaneously. The nuclease may then be expressed by the target cell and may function in concert with the gRNA, and optionally the DNA repair template, to edit a gene of the target cell.

The method of delivering two or more nucleic acids to a target cell may be useful where the nucleic acids are nucleic acids which encode for different peptides or proteins which are interrelated such as subunits or components of the same molecule, or molecules that have an interlinked operation, such as components of the same biological pathways, or exhibit a ligand: receptor binding relationship. As shown herein, EVs loaded with cargo encoding different component peptides on separate nucleic acids affords superior expression as compared to encoding the peptides on a single nucleic acid (e.g. a bicistronic vector), as well as delivering those cargo separately in different EVs. In some cases the target cell will produce a peptide(s) encoded by a nucleic acid of the cargo. In some cases the target cell will produce and secrete the peptide(s) encoded by a nucleic acid of the cargo. In some cases the target cell will produce different polypeptide components of a molecule, which assemble within the target cell to form the complete molecule.

The method is also useful where it is important to understand which target cells have taken up a cargo. In such cases the cargo may comprise a reporter gene or encodes a molecule that is readily detectable. In some cases the cargo may comprise a reporter gene or detectable molecule and at least one nucleic acid of interest (e.g. a plasmid, an ASO, an siRNA or components of a CRISPR gene editing system). The reporter gene or detectable molecule will be delivered to the target cell alongside the nucleic acid(s) of interest and enables the detection of cells which have taken up the cargo and therefore contain the nucleic acid(s) of interest.

In some cases the method of delivering two or more nucleic acids to a cell is performed in vitro or ex vivo. In some cases the method of delivering two or more nucleic acids to a cell is performed in vivo. In some cases the target cell has been isolated from a subject, e.g. a human subject. In some cases the target cell is present in a subject, e.g. a human subject. In some cases the subject has cancer. In some cases the subject has a monogenic disease. In some cases, the subject has polygenic disease.

In some cases the target cell is a cancer cell or tumour cell. In some cases the target cell is an immune cell. In some cases the target cell comprises a genetic mutation. In some cases the target cell comprises a faulty gene. In some cases, the target cell comprises a loss of function mutation. In some cases, the target cell comprises a gain of function mutation. In some cases the target cell has a mutation leading to overexpression or underexpression of a protein.

Methods of delivering two or more nucleic acids to a cell comprise a step of contacting a target cell with an EV, wherein the EV is loaded with a cargo comprising at least two different nucleic acids. The target cell and the EV are contacted for a sufficient time, and under conditions suitable for the target cell to take up the EV.

The EV may be incubated with the target cell. The terms “incubating”/“incubation”/“incubate” are used herein to refer to placing the target cell(s) and EV(s) loaded with a cargo together at a suitable temperature and for a suitable time such that the EV(s) are taken up, i.e. assimilated, incorporated or taken in, by the target cell(s). These terms are also used herein to refer to bringing the target cell(s) and loaded EV(s) into sufficient contact that the target cell(s) take up, i.e. assimilate, incorporate or take in, the EV(s) and/or the cargo e.g. exogenous nucleic acid, e.g. during or after incubation. Incubation may produce the target cell(s) described herein that comprise or contain at least one EV and/or cargo. The target cell(s) may be produced during and/or after incubation. Incubation may involve culturing the target cells, or populations thereof, in vitro/ex vivo in cell culture medium comprising the cargo-loaded EVs. Incubation may be performed at a temperature close to body temperature of a mammal, e.g. at one or more of at least 35.0° C., at least 35.5° C., at least 36.0° C., at least 36.1° C., at least 36.2° C., at least 36.3° C., at least 36.4° C., at least 36.5° C., at least 36.6° C., at least 36.7° C., at least 36.8° C., at least 36.9° C., at least 37.0° C., at least 37.1° C., at least 37.2° C., at least 37.3° C., at least 37.4° C., and/or at least 37.5° C. In some cases the incubation is performed at two or more temperatures, e.g. as above. In some cases the incubation is performed at a single temperature. In some cases the incubation is performed at human body temperature. In some cases, incubation is performed at at least 37.0° C. In some cases, incubation is performed at 37.0 ° C. Incubation may be repeated on the same cells.

Incubation may comprise controlling the CO₂ level of the cell culture. Incubation comprising controlled CO₂ can control the pH of the incubated mixture. In some cases the CO₂ level of the incubating mixture is maintained at or close to the CO₂ level of blood, e.g. mammalian blood. In some cases incubation is performed at one or more of at least 4.0%, at least 4.1%, at least 4.2%, at least 4.3%, at least 4.4%, at least 4.5%, at least 4.6%, at least 4.7%, at least 4.8%, at least 4.9%, at least 5.0%, at least 5.1%, at least 5.2%, at least 5.3%, at least 5.4%, at least 5.5%, at least 5.6%, at least 5.7%, at least 5.8%, at least 5.9% and/or at least 6.0% CO₂. In some cases incubation is performed at one or more of at least 30 mmHg, at least 31 mmHg, at least 32 mmHg, at least 33 mmHg, at least 34 mmHg, at least 35 mmHg, at least 36 mmHg, at least 37 mmHg, at least 38 mmHg, at least 39 mmHg, at least 40 mmHg, at least 41 mmHg, at least 42 mmHg, at least 43 mmHg, at least 44 mmHg, and/or at least 45 mmHg CO₂.

In some cases incubation is performed at at least 5% CO₂. In some cases incubation is performed at about 5% CO₂. In some cases incubation is performed at 5% CO₂. In some cases incubation is performed at at least 38 mmHg CO₂. In some cases incubation is performed at about 38 mmHg CO₂. In some cases incubation is performed at 38 mmHg CO₂. In some cases incubation is performed in a humidified environment, e.g. in a humidified incubator.

Incubation may be performed for a length of time such that the EVs are taken up by the target cells. Incubation may be performed, e.g. at a combination of temperature and CO₂ level e.g. as above, for one of 12, 24, 36, 48, 60 or 72 hours. In some cases incubation is performed for at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, or at least 72 hours.

In some cases incubation is performed for at least 36 or at least 48 hours. In some cases incubation is performed for 48 hours. The methods described herein may comprise one or more steps of washing the target cells after incubation, e.g. to remove any non-assimilated EVs. Washing may be performed using PBS and centrifugation, e.g. at 4° C.

The methods described herein may comprise an incubation step comprising any combination of temperature, CO₂ level, and/or time, e.g. as described above. In some cases, incubation is performed at 37° C.at 5% CO₂ for 48 hours.

Incubation may be performed in any suitable medium, e.g. a cell culture medium.

In some cases incubation comprises agitating the mixture for some or all of the incubation time.

The method may include a step of loading the EV with a nucleic acid cargo, e.g. as described herein. This step may be performed prior to incubating the EV with the target cell. This step may be performed separately to incubating the EV with the target cell.

In some cases the method does not include a step of loading the EV with a nucleic acid cargo, e.g. the target cells are incubated with an EV that has been pre-loaded with a nucleic acid cargo.

In some cases the methods of delivering/transfecting a target cell(s) with exogenous nucleic acid described herein do not comprise contacting the target cell(s) with transfection reagents (although the EVs themselves may be/have been loaded with nucleic acid cargo using e.g. transfection reagents).

### Methods of Treatment and Uses of Extracellular Vesicles

Extracellular vesicles disclosed herein are useful in methods of treatment. As demonstrated herein, cells transfected with RBCEVs co-loaded with two or more nucleic acids (co-loading) resulted in a much higher level of expression from all cargoes, as compared to cells transfected with RBCEVs loaded individually with the respective cargoes and mixed equally thereafter (co-transfecting) which showed a higher level of expression heterogeneity. The effects of co-loading on co-expression from both transgenes was more pronounced than what can be achieved through the use of bicistronic vectors. Thus, extracellular vesicles loaded with cargo, wherein the cargo comprises at least two different nucleic acids, according to the invention are particularly useful in methods of treatment.

Extracellular vesicles are useful in methods of treatment that are known to benefit from the administration of multiple nucleic acid components, such as methods of treatment by gene editing (which require the administration of multiple components of a gene editing system) or vectorised antibody (which require the administration of multiple components of an antibody or antigen binding fragment thereof). Some methods involve the suppression of an endogenous nucleic acid and simultaneous expression of an exogenous nucleic acid. For example, such methods may involve the suppression of a “faulty” endogenous nucleic acid such as a gain-of-function mutation, and expression of an exogenous functional copy of that nucleic acid. The treatment may involve the administration of nucleic acids to target two or more endogenous nucleic acids, such as in the treatment of a polygenic disease.

In particular, the methods are useful for treating a subject suffering from a disorder associated with a target gene, the method comprising the step of administering an effective amount of a modified extracellular vesicle to said subject, wherein the modified extracellular vesicle comprises a binding molecule on its surface and encapsulates a non-endogenous substance for interacting with the target gene in a target cell. The non-endogenous substance may be a nucleic acid for said treatment.

The extracellular vesicles disclosed herein are particularly useful for the treatment of a genetic disorder, inflammatory disease, cancer, autoimmune disorder, cardiovascular disease or a gastrointestinal disease. In some cases, the disorder is a genetic disorder selected from thalassemia, sickle cell anemia, or genetic metabolic disorder. In some cases, the extracellular vesicles are useful for treating a disorder of the liver, bone marrow, lung, spleen, brain, pancreas, stomach or intestine.

In certain aspects, the extracellular vesicles are useful for the treatment of cancer. Extracellular vesicles disclosed herein may be useful for inhibiting the growth or proliferation of cancerous cells. The cancer may be a liquid or blood cancer, such as leukemia, lymphoma or myeloma. In other cases, the cancer is a solid cancer, such as breast cancer, lung cancer, liver cancer, colorectal cancer, nasopharyngeal cancer, kidney cancer or glioma. In some cases, the cancer is located in the liver, bone marrow, lung, spleen, brain, pancreas, stomach or intestine.

The target cell depends on the disorder to be treated. For example, the target cell may be a breast cancer cell, a colorectal cancer cell, a lung cancer cell, a kidney cancer cell or the like. The cargo may comprise a nucleic acid for inhibiting or enhancing the expression of the target gene, or performing gene editing to silence the particular gene.

Extracellular vesicles and compositions described herein may be administered, or formulated for administration, by a number of routes, including but not limited to systemic, intratumoral, intraperitoneal, parenteral, intravenous, intra-arterial, intradermal, subcutaneous, intramuscular, oral and nasal. Preferably, the extracellular vesicles are administered by a route selected from intratumoral, intraperitoneal or intravenous. The medicaments and compositions may be formulated in fluid or solid form. Fluid formulations may be formulated for administration by injection to a selected region of the human or animal body.

The extracellular vesicle may comprise a therapeutic cargo. The therapeutic cargo may be a non-endogenous substance for interacting with a target gene in a target cell. The therapeutic cargo may be a vectorised antigen-binding molecule.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Extracellular vesicles may be administered alone or in combination with other treatments, either simultaneously or sequentially dependent upon the condition to be treated.

Extracellular vesicles loaded with a cargo as described herein may be used to deliver that cargo to a target cell. In some cases, the method is an in vitro method. In particularly preferred in vitro methods the cargo is a labelling molecule or a plasmid.

The subject to be treated may be any animal or human. The subject is preferably mammalian, more preferably human. The subject may be a non-human mammal, but is more preferably human. The subject may be male or female. The subject may be a patient. Therapeutic uses may be in humans or animals (veterinary use).

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1

RBCEVs can be simultaneously loaded with two or more nucleic acid cargoes, and these co-loaded EVs are very efficient in delivering their payload to target cells. This has been demonstrated by assessing the expression pattern and levels of fluorescent reporter genes by fluorescence microscopy and flow cytometry analysis, gene transcript levels by qRT-PCR, and expression levels of a therapeutic antibody. Cells transfected with RBCEVs co-loaded with two or more nucleic acids (co-loading) resulted in a much higher level of expression from all cargoes, as compared to cells transfected with RBCEVs loaded individually with the respective cargoes and mixed equally thereafter (co-transfecting) which showed a higher level of expression heterogeneity. The effects of co-loading on co-expression from both transgenes was more pronounced than what can be achieved through the use of bicistronic vectors. These findings suggest that co-loaded RBCEVs are promising vehicles for the delivery of multiple cargoes, with significant implications in the areas of vectorized antibodies and genome editing.

### Methods

### Materials and Reagents

Plasmids (CMV-copGFP, CMV-tdTomato, CAG-Cre, CAG-hFIX, CAG-Luciferase, CAG-LC, CAG-HC, CAG-LC-IRES-HC, CAG-LC-P2A-HC) were custom synthesized. The full-length heavy chain (HC) and light chain (LC) of trastuzumab were obtained from the drug bank database (Accession Number: DB00072). tdTomato mRNA was custom synthesized from TriLink Biotechnologies. 293T and HepG2 cells were purchased from ATCC and cultured in Dulbecco's Modified Eagle's Medium containing 10% fetal bovine serum, in a 37° C. CO₂ incubator.

Purification and Quantification of EVs from Human RBCs

Whole blood samples were obtained through Innovative Research, Inc from healthy donors with informed consents. RBCs were separated from plasma and white blood cells by using centrifugation and leukodepletion filters (Terumo Japan). Isolated RBCs were diluted in PBS and treated with 10 mM calcium ionophore (Sigma-Aldrich) overnight. To purify EVs, RBCs and cell debris were removed by centrifugation at 600×g for 60 min, 1,600×g for 30 min, and 4,000×g for 30 min at 4° C. EVs were pelleted at 15,000×g for 180 min at 4° C., resuspended in PBS and passed through a 0.45 μm filter. EVs were washed with 4 diavolumes of PBS and concentrated by tangential flow filtration (Pall Minimate). Purified EVs were stored at −80° C. EVs were quantified by assessing their hemoglobin content using the Hemoglobin Assay Kit (Abcam).

### Nucleic Acid Loading of RBCEVs and in Vitro Transfection

1 μg of mRNA or DNA was added to 7 μl of chemical-based transfection reagent in Opti-MEM (ThermoFisher) and incubated at room temperature for 10 minutes to facilitate complex formation. The mixture was added to 50 μg of washed RBCEVs and mixed gently. The reaction was incubated at room temperature for 1 hour with gentle rotation. Thereafter, loaded RBCEVs were pelleted at 15,000×g and washed with PBS.

For co-loading of multiple nucleic acids, plasmids and/or mRNA were first mixed at an equimolar ratio before the chemical-based transfection reagent was added. After the complexation, the mixture was added to RBCEVs and incubated at room temperature for 1 hour with gentle rotation. Loaded RBCEVs were pelleted at 15,000×g and washed with PBS.

For the co-transfection of RBCEVs, 2 or more plasmids or mRNA were loaded to RBCEVs in separate tubes, and after PBS washing the nucleic acids in these loaded RBCEVs were quantified. Loaded RBCEVs were mixed at an equimolar ratio of their respective nucleic acids before cell transfection.

50,000 293T or HepG2 cells were seeded in each well of a 24-well plate one day prior to transfection. On the day of transfection, loaded RBCEVs were treated to cells at an equimolar amount of the nucleic acid. The 24-well plate was swirled gently to distribute the particles evenly in the well.

### Assessment of Gene Expression

For fluorescent reporter genes, gene expression was assessed through microscopic imaging and flow cytometry. For microscopic imaging, Epi-fluorescent images of 293T or HepG2 cells were taken 48 hours after transfection using a Nikon Eclipse Ts2 inverted microscope. Images were analyzed using Fiji, an open-source platform for biological-image analysis (Nature methods 9(7): 676-682, PMID 22743772). Colocalization analysis of a 2-color channel image was performed using the Fiji plugin Coloc 2. The Pearson's correlation coefficient was selected to measure the degree of correlation of colocalizing signals, where the result is 1.0 for perfect correlation and 0.0 for no correlation.

For flow cytometry analysis, 293T and HepG2 cells were harvested with 0.05% trypsin 48 hours after transfection. Harvested cells were washed with PBS and resuspended in FACS buffer (PBS containing 1% FBS). Flow cytometry of cells was performed using the CytoFLEX S Flow Cytometer (Beckman Coulter) and analyzed using FlowJo V10 (FlowJo, USA). Cells were initially gated based on FSC-A vs. SSC-A to exclude the debris and dead cells (low FSC-A). Cells were further gated based on FSC-width vs. FSC-height, to exclude doublets and aggregates. Using untreated cells as controls, GFP-positive cells were gated in the FITC channel and tdTomato-positive cells were gated in the PE channel. Percentage of GFP and/or tdTomato positive cells were assessed.

For other plasmids (non-fluorescent), gene expression was measured by transcript levels determined by qRT-PCR. Total RNA from cells transfected with nucleic acid loaded RBCEVs was extracted using TRIzol (ThermoFisher) and were converted to cDNA using the LunaScript RT SuperMix Kit (New England Biolabs) following the manufacturer's protocol. qPCR was performed on cDNA samples to determine transcript levels of transgenes delivered to cells. Expression was normalized to GAPDH.

### Quantification of Trastuzumab

Levels of trastuzumab in cell culture supernatant was determined by the anti-HER2 ELISA kit (ab237645, Abcam) following the manufacturer's protocol. This detection method was chosen because a positive signal only occurs in the presence of an intact whole antibody that includes both heavy and light chains.

### Loading Efficiency

DNA or RNA was quantified by PicoGreen or RiboGreen assay. Loading efficiency was calculated based on starting amount of nucleic acid added to the loading reaction and final amount of nucleic acid recovered after the loading reaction.

### Labeling of DNA and Flow Cytometry Analysis of RBCEVs

DNA was labeled with a fluorophore (MFP488 or Cy5) using the Label IT Nucleic Acid Labeling Kit (Mirus Bio) following the manufacturer's protocol. RBCEVs were loaded with MFP488 or Cy5-labeled DNA and washed with PBS. Loaded RBCEVs were analyzed by flow cytometry using the CytoFLEX S Flow Cytometer (Beckman Coulter) and analyzed using FlowJo V10 (FlowJo, USA). RBCEVs were gated based on VSSC-A vs. FSC-A using 100 nm and 200 nm sizing latex beads as a sizing reference. Using RBCEVs loaded with unlabeled DNA as controls, MFP488-positive RBCEVs were gated in the FITC channel and Cy5-positive RBCEVs were gated in the APC channel.

### Statistical Analysis

The results are expresses as mean±standard deviation (SD). Data were analyzed by Student's t-test, one-way ANOVA, or Pearson's correlation. Difference between means was considered statistically significant at p<0.05.

### Results

RBCEVs can be Loaded with 2 or More Plasmids Simultaneously

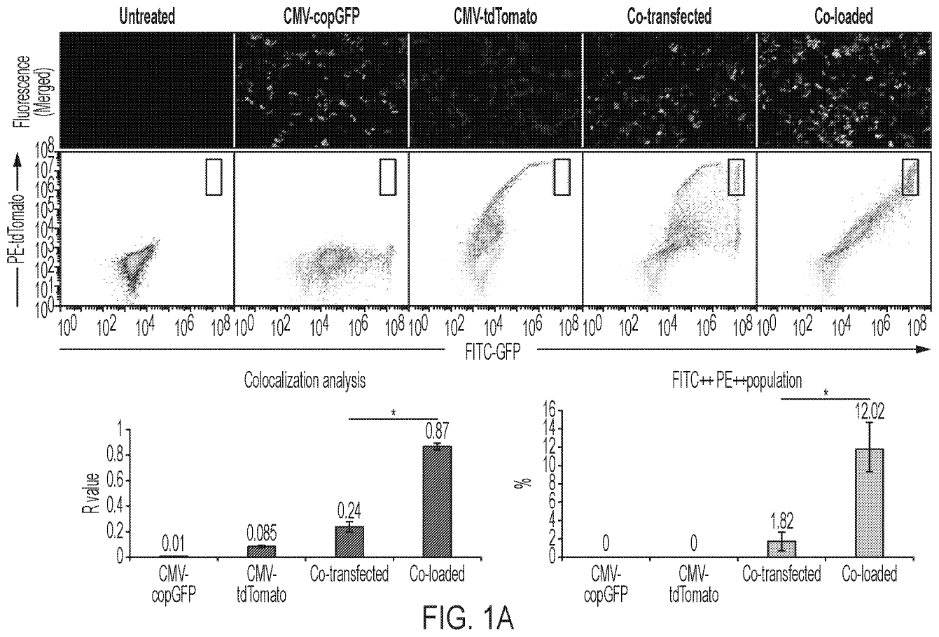

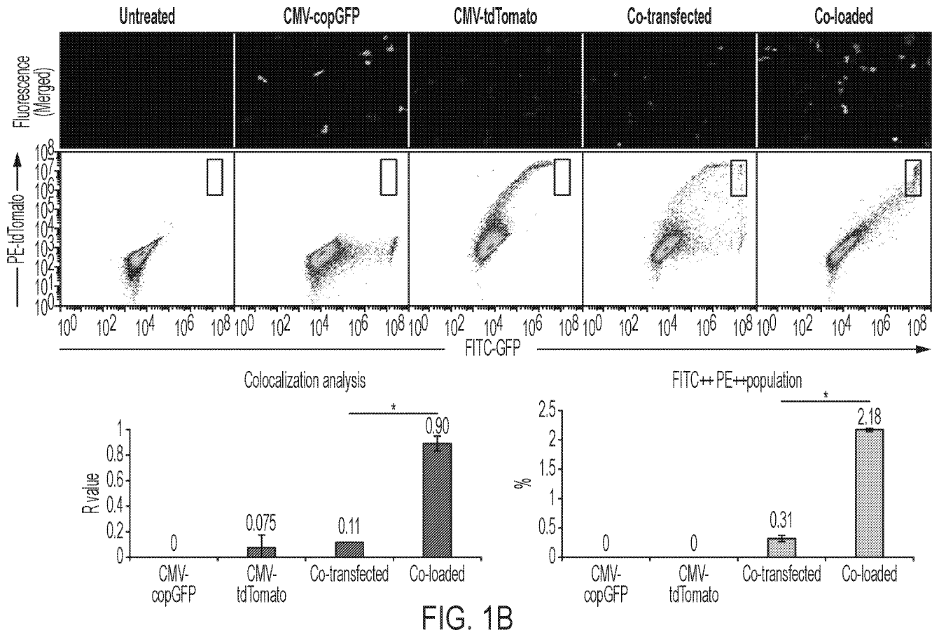

To assess the transfection efficiency of 2 plasmids by RBCEVs, plasmid constructs with different fluorescent reporter genes under the same CMV promoter were designed (CMV-copGFP and CMV-tdTomato). RBCEVs were loaded with single plasmids and co-transfected, or simultaneously loaded with both plasmids at an equimolar ratio. Loaded RBCEVs were transfected to 293T and HepG2 cells at equimolar DNA amount. 0.084 pmol of DNA was added to each well of a 24-well plate containing 50,000 cells. 48 hours after transfection, cells were image by fluorescence microscopy and analyzed by flow cytometry (FIG. 1A and 1B).

Colocalization analysis of 2-color channel images of 293T and HepG2 cells using the Pearson's correlation coefficient was performed to measure the degree of correlation of colocalizing signals. Cells that were treated with RBCEVs co-loaded with both plasmids showed a significantly higher correlation of colocalizing signals of the 2-color channels. The increased correlation coefficient in the co-loaded groups suggests the increased transfection efficiency and expression of both plasmids in cells.

Cells were harvested and analyzed by flow cytometry. Using untreated cells as controls, GFP-positive cells were gated in the FITC channel and tdTomato-positive cells were gated in the PE channel, and the percentage of GFP and/or tdTomato positive cells were assessed. Increased transgene co-expression of both GFP and tdTomato was observed in cells that were transfected with co-loaded EVs. Dot plots of cells in co-transfected groups showed a heterogenous population of cells expression of GFP or tdTomato, whereas a more linear pattern was observed in the co-loaded group. These results suggest a highly correlated delivery of both plasmids to cells by simultaneously loading RBCEVs with both plasmids.

Plasmids Co-Loaded in RBCEVs were More Efficiently Co-Expressed as Compared to Those Loaded Separately and Co-Transfected, or Bicistronic Vectors

We have assessed the RBCEV loading of plasmids encoding for therapeutic antibodies such as the DNA-derived humanized monoclonal antibody trastuzumab (Herceptin™, Genentech). A highly specific ELISA kit (ab237645, Abcam) designed for the determination of anti-HER2 (trastuzumab) was used for the detection and quantification of the expression of the whole antibody molecule. In this assay the ELISA plates are coated with the antigen, a recombinant human HER2 fragment. The anti-HER2 antibody (trastuzumab) binds to the antigen and is detected with an anti-human Fc antibody conjugated with HRP probe. Using this detection method, we are able to detect the whole antibody molecule that is assembled from the light chain and heavy chain components.

Plasmid constructs were designed to contain either the trastuzumab light chain (CAG-LC), the heavy chain (CAG-HC), or both light and heavy chain sequences in a bicistronic vector with either the IRES (CAG-LC-IRES-HC) or P2A (CAG-LC-P2A-HC) sequence. To assess the delivery and transfection efficiency of RBCEVs, RBCEVs were loaded with single plasmids encoding for the trastuzumab light chain, heavy chain, or single bicistronic vectors with the IRES or P2A sequence. In the co-loading group, RBCEVs were simultaneously loaded with plasmids encoding for the trastuzumab light chain and heavy chain at an equimolar ratio.

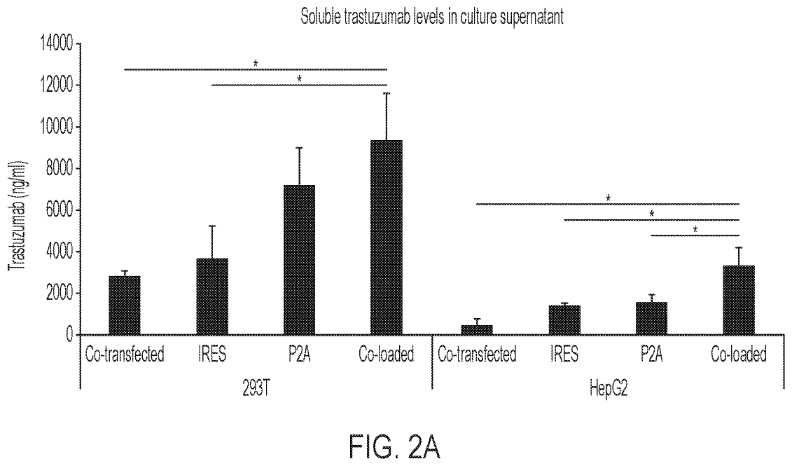

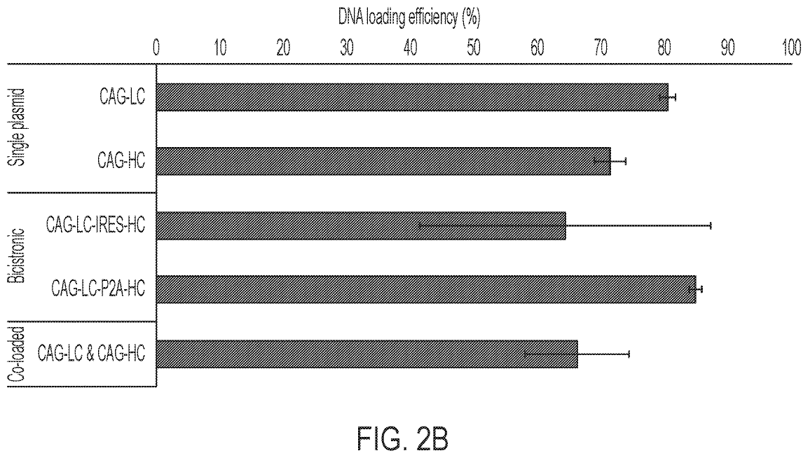

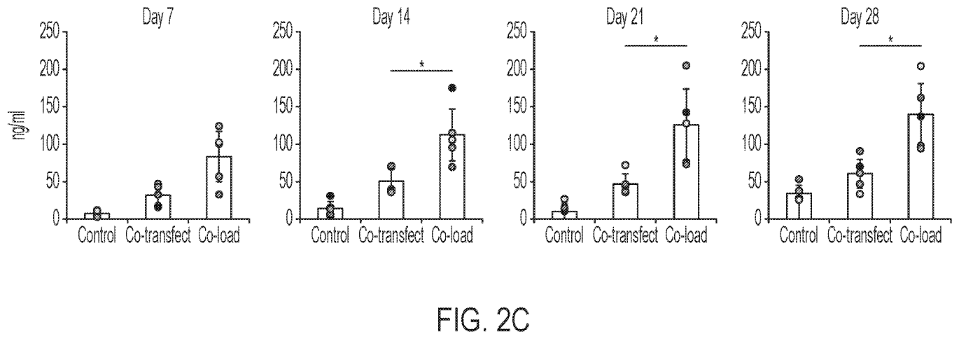

Loaded RBCEVs were transfected to 293T and HepG2 cells at equimolar DNA amount. 0.084 pmol of DNA was added to each well of a 24-well plate containing 50,000 cells. 48 hours after transfection, the expression of trastuzumab in the cell culture supernatant were quantified by anti-HER2 ELISA. Expression of trastuzumab was highest in cells treated with RBCEVs co-loaded with the trastuzumab light chain and heavy chain plasmids at an equimolar ratio, followed by RBCEVs loaded with the single bicistronic vectors (FIG. 2A). Cells that were co-transfected with RBCEVs that were loaded with single plasmids encoding for the trastuzumab light chain or heavy chain showed least expression of the antibody whole molecule. These results suggest that transfection efficiency of 2 plasmids is improved significantly when plasmids are co-loaded with RBCEVs. The difference in the expression of the antibody whole molecule demonstrates that the difference in expression of either plasmids, as assembly of both the light chain and heavy chain components are necessary to be detected by the anti-HER2 ELISA method. No significant difference was observed on loading efficiency of different plasmids involved (FIG. 2B).

In another experiment, RBCEVs simultaneously loaded with plasmids encoding for the trastuzumab light chain and heavy chain or separately loaded with single plasmids encoding for the trastuzumab light chain or heavy chain were administered to SCID mice via intravenous tail-vein injection at a plasmid dose of 4 mg/kg. Serum samples were collected weekly from day 7 post injection and the expression of the whole antibody molecule was quantified using the anti-HER2 (trastuzumab) ELISA kit (ab237465, Abcam). The expression of trastuzumab was highest in animals injected with the RBCEVs co-loaded with both the light chain and heavy chain plasmids compared to animals that were injected with RBCEVs that were separately loaded with the light chain and heavy chain plasmids (FIG. 2C). These results demonstrate that plasmids co-loaded in RBCEVs are more efficiently co-expressed in vivo, as compared to RBCEVs loaded separately and injected as a mixture.

Thus, among other things, the present Example demonstrates efficient co-loading with at least two different nucleic acid cargos (in this case at least two different plasmids), and furthermore documents surprising results attributable to such co-loading (e.g., as compared with co-administration of RBCEVs separately loaded with each of the same nucleic acid cargos).

RBCEVs can be Co-Loaded with 3 Plasmids

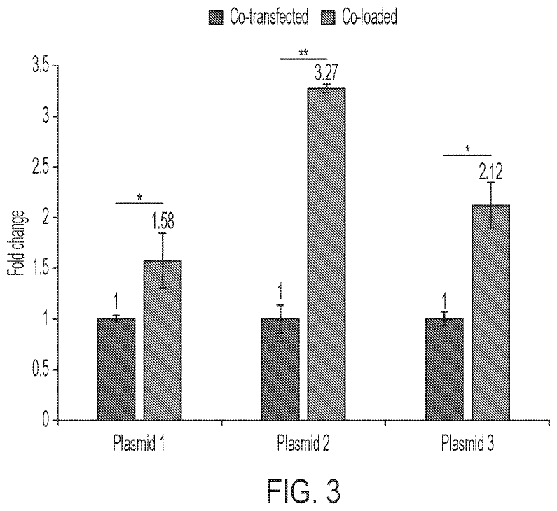

To assess the transfection efficiency of 3 plasmids by RBCEVs, plasmid constructs encoding unique transgenes namely Cre recombinase, human factor IX and firefly luciferase under the same CAG promoter were designed. RBCEVs were loaded with the 3 different plasmids either separately in different loading reactions, or by co-loading plasmids at an equimolar ratio in a mixture. In total 0.06 pmol of DNA was transfected to 293T cells. 48 hours after transfection, total RNA was extracted from cells and gene expression was measured by transcript levels determined by qRT-PCR. The results showed increased levels of transcripts from all 3 plasmids when cells were transfected with co-loaded RBCEVs when compared to co-transfection (FIG. 3).

RBCEVs can be Co-Loaded with Different Types of Nucleic Acids

To assess the ability of RBCEVs to be co-loaded with different nucleic acid types, RBCEVs were co-loaded with DNA encoding for copGFP (CMV-copGFP) and mRNA encoding for tdTomato at an equimolar ratio of 1:1 or 1:2 (DNA:RNA), or loaded separately using a chemical-based method. 0.114 pmol of nucleic acid was added to each well of a 24-well plate containing 50,000 293T cells. 48 hours after transfection, cells were imaged by fluorescence microscopy and flow cytometry analysis.

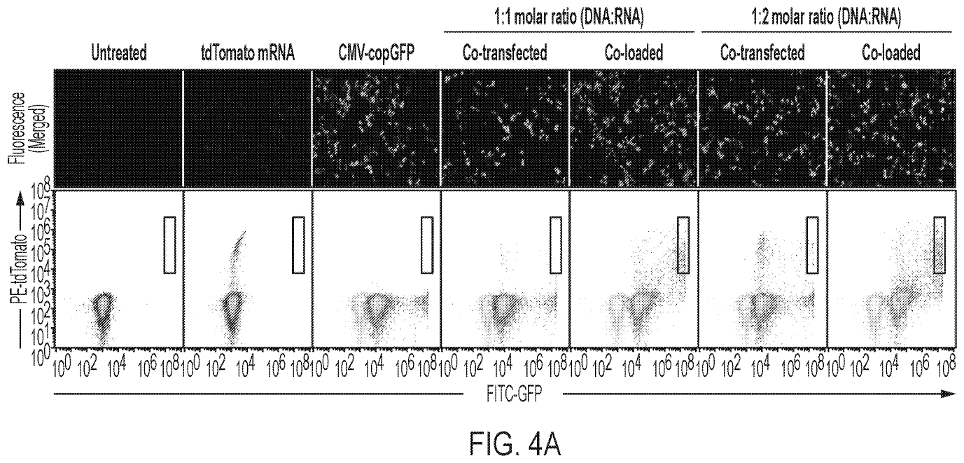

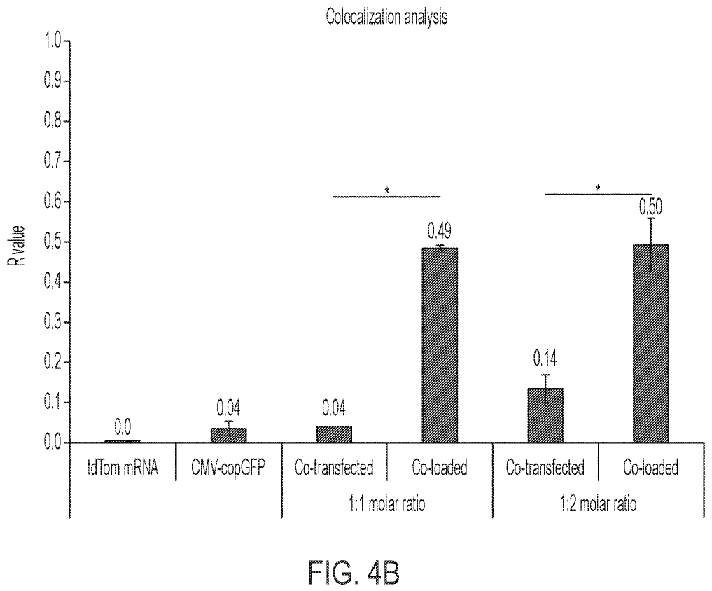

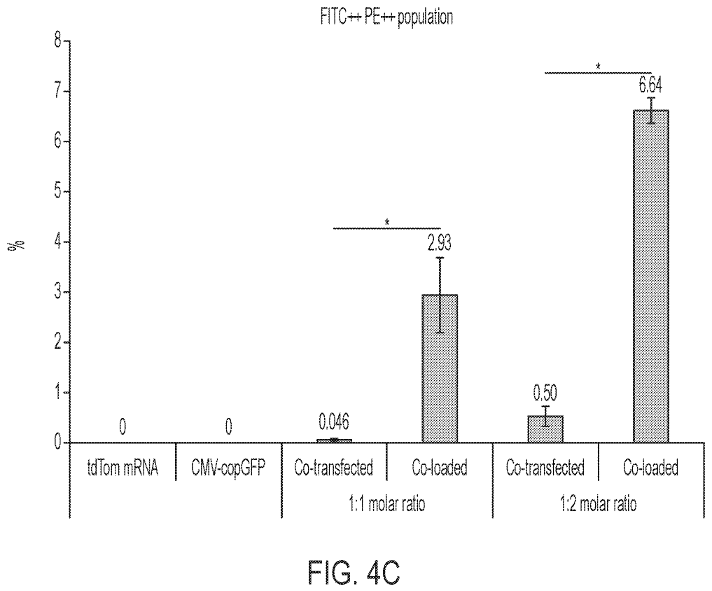

Cells that were treated with RBCEVs co-loaded with both copGFP DNA and tdTomato mRNA showed a significantly higher correlation of colocalizing signals of the 2-color channels in both 1:1 and 1:2 molar ratio groups (FIG. 4). The increased correlation coefficient in the co-loaded groups suggests the increased transfection efficiency and expression of both nucleic acids in single cells. Flow cytometry analysis showed a similar pattern, where an increase in percentage of GFP and tdTomato positive 293T cells was observed in co-loaded groups. The expression of DNA and mRNA co-loaded in RBCEVs was more efficient than the co-transfection of RBCEVs that were separately loaded with DNA or mRNA.

### RBCEV Loading of Fluorescently-Labelled DNA

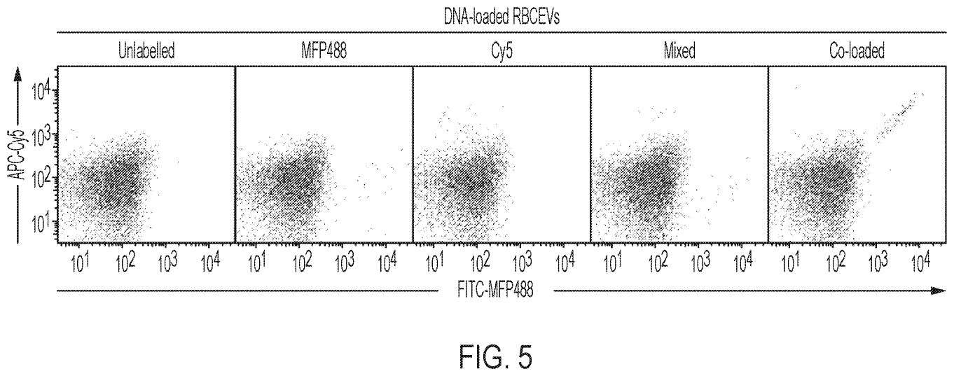

DNA was labeled with a fluorophore (MFP488 or Cy5) using the Label IT Nucleic Acid Labeling Kit (Mirus Bio) following the manufacturer's protocol. RBCEVs were loaded with MFP488 or Cy5-labeled DNA, or simultaneously loaded with equal amounts of MFP488 and Cy5-labeled DNA. Loaded RBCEVs were analyzed by flow cytometry using the CytoFLEX S Flow Cytometer (Beckman Coulter) and analyzed using FlowJo V10 (FlowJo, USA). RBCEVs were gated as a population with a size ranging between 100 nm and 200 nm using latex bead standards as a sizing reference. Using RBCEVs loaded with unlabeled DNA as controls, MFP488-positive RBCEVs were gated in the FITC channel and Cy5-positive RBCEVs were gated in the APC channel. Increased co-expression of both fluorescent labels was observed in RBCEVs that were simultaneously loaded with equal amounts of MFP488 and Cy5-labeled DNA compared to the mixed population of RBCEVs loaded separately with MFP488-labeled DNA or Cy5-labeled DNA (FIG. 5). These results demonstrate that a single RBCEV can be loaded with at least two different fluorescently-labelled DNA molecules concurrently using a co-loading method as described herein

For standard molecular biology techniques, see Sambrook, J., Russel, D.W. Molecular Cloning, A Laboratory Manual. 3 ed. 2001, Cold Spring Harbor, New York: Cold Spring Harbor Laboratory Press

### Example 2

The present Example demonstrates that co-loading of RBCEVs with double-stranded oligonucleotides can increase transgene expression when delivered in vitro or in vivo while also suppressing the immune response to the transgene. Specifically, transgene expression and inflammatory cytokines were assessed after co-loaded RBCEVs were delivered to multiple mouse strains and multiple human cell lines of different tissue origin.

Exogenous DNA found in the cytoplasm can activate the innate immune response by DNA-activated signaling pathways such as the cGAS-STING pathway, resulting in the production of pro-inflammatory cytokines. The present invention proposes that co-loading of red blood cell derived extracellular vesicles (RBCEVs) with oligonucleotides (and specifically with oligonucleotides that can mimic or “decoy” some aspect of cellular machinery associated with sensing foreign nucleic acids) can enhance activity of a co-loaded nucleic acid payload relative to the corresponding RBCEVs loaded with payload alone. The present Example documents such enhancement, both in vivo and in vitro. The present Example further demonstrates that, in addition to the enhancement of transgene expression, co-loading RBCEVs with such oligonucleotides (e.g., decoy oligonucleotides) can act to mitigate cellular innate immune response by reducing the induction of Type I interferons in vivo.

In the present disclosure, the term “decoy oligonucleotide” (abbreviated as “ODN”) represents a class of oligonucleotides that can block the transcriptional activity of immune response and signaling-related transcription factors such as NF-κB, as an effective strategy to counter the surveillance of the cGAS-STING pathway. For example, oligonucleotides containing the NF-κB consensus sequence can block or reduce (e.g., by competition) NF-κB binding to its site(s) in promoter region(s) of its target genes. Such blocking reduces NF-κB's ability to activate those target genes, so that expression of those genes (and/or of one or more other downstream genes is reduces, and so also is the release of inflammatory cytokines.

### Materials and Methods

Purification and Quantification of EVs from Human RBCs

Whole blood samples were obtained through Innovative Research, Inc from healthy donors with informed consent. RBCs were separated from plasma and white blood cells by using centrifugation and leukodepletion filters (Terumo Japan). Isolated RBCs were diluted in PBS and treated with 10 mM calcium ionophore (Sigma-Aldrich) overnight. To purify EVs, RBCs and cell debris were removed by centrifugation at 600×g for 60 min, 1,600×g for 30 min, and 4,000×g for 30 minutes at 4° C. EVs were pelleted at 15,000×g for 180 minutes at 4° C., resuspended in PBS and passed through a 0.45 μm filter. EVs were washed with 4 diavolumes of PBS and concentrated by tangential flow filtration (Pall Minimate). Purified EVs were stored at −80° C. EVs were quantified by assessing their hemoglobin content using the Hemoglobin Assay Kit (Abcam).

### Plasmids

DNA plasmids (CMV-copGFP, LSP-FIX-HiBit, CAG-LC, CAG-HC) were custom synthesized. The full-length heavy chain (HC) and light chain (LC) of trastuzumab were obtained from the drug bank database (Accession Number: DB00072).

### Oligonucleotides

All oligonucleotide designs were synthesized by IDT and purified by either standard desalting or by HPLC.

Decoy to the NF-κB consensus sequence (ODN)—a 22-bp synthetic oligonucleotide containing the 11-bp NF-κB binding sequence: GGGGACTTTTCC (De Stefano et al., “A decoy oligonucleotide to NF-κB delivered through inhalable particles prevents LPS-induced rat airway inflammation.” American Journal of Respiratory Cell and Molecular Biology 49.2 (2013)).

5′-GATCGAGGGGACTTTCCCTAGC-3′
3′-CTAGCTCCCCTGAAAGGGATCG-5′

Scrambled NF-κB consensus sequence (SCD)—a scrambled sequence based on the 22-bp ODN, generated using the program from Stothard P (2000) The Sequence Manipulation Suite: JavaScript programs for analyzing and formatting protein and DNA sequences. Biotechniques 28:1102-1104. A scrambled sequence, as described herein, contains the same residues but in a different order as compared to a reference sequence (e.g., NF-κB decoy oligonucleotide). In some cases, a scrambled sequences changes the function and/or structure of an oligonucleotide. In some cases, a scrambled sequences does not change the function and/or structure of an oligonucleotide.

5′-ATCTGGCGGTCCAGTTAGAGCC-3′
3′-TAGACCGCCAGGTCAATCTCGG-5′

### Nucleic Acid Loading of RBCEVs

1 μg of DNA was added to 20 μg of washed RBCEVs and 7 μl of a chemical-based transfection reagent in Opti-MEM (ThermoFisher) and mixed gently. The reaction was incubated at room temperature for 1 hour with gentle rotation. Thereafter, loaded RBCEVs were pelleted at 15,000×g and washed with PBS. For the co-loading of RBCEVs with DNA plasmids and oligonucleotides, plasmids and oligonucleotides were first mixed with RBCEVs before the chemical-based transfection reagent was added. After the addition of the chemical-based transfection reagent, the reaction was incubated at room temperature for 1 hour with gentle rotation. Loaded RBCEVs were pelleted at 15,000×g and washed with PBS. DNA was quantified by gel densitometry. Loading efficiency was calculated based on starting amount of nucleic acid added to the loading reaction and final amount of nucleic acid recovered after the loading reaction.

### Cell Lines and in Vitro Transfection

HepG2 cells were purchased from ATCC, and Huh-7 cells were purchased from Elabscience. Cells were cultured in DMEM containing 10% FBS and 1% Penicillin-Streptomycin. THP-1 cells were purchased from ATCC and cultured in RPMI 1640 media containing 10% FBS and 1% Penicillin-Streptomycin. All cell lines were maintained at 37° C.in a 5% CO₂ incubator.

For in vitro transfection, 50,000 cells were seeded in each well of 24-well plate and incubated overnight. On the day of transfection, loaded RBCEVs were delivered to cells based on the quantification of the DNA plasmid. Cells were incubated for 24 or 48 hours and analyzed for transgene expression.

### Assessment of Gene Expression

For fluorescent reporter genes, gene expression was assessed through microscopic imaging and flow cytometry. For microscopic imaging, Epi-fluorescent images of HepG2 cells were taken 24 or 48 hours after transfection using a Nikon Eclipse Ts2 inverted microscope. For flow cytometry analysis, HepG2 cells were harvested with 0.05% trypsin 24 or 48 hours after transfection. Harvested cells were washed with PBS and resuspended in FACS buffer (PBS containing 1% FBS). Flow cytometry of cells was performed using the CytoFLEX S Flow Cytometer (Beckman Coulter) and analyzed using FlowJo V10 (FlowJo, USA). Cells were initially gated based on FSC-A vs. SSC-A to exclude the debris and dead cells (low FSC-A). Cells were further gated based on FSC-width vs. FSC-height, to exclude doublets and aggregates. Using untreated cells as controls, GFP-positive cells were gated in the FITC channel and PI-stained cells were gated in the PE channel. Percentage of GFP and/or PI-stained cells were assessed.

Nano-Glo HiBiT Lytic Detection System to quantify HiBiT-tagged protein expression—For the quantification of HiBiT-tagged proteins, samples were diluted 100-fold using PBS. 100 μl of the diluted samples were mixed with an equal volume of Nano-Glo HiBiT Lytic Reagent (Promega), consisting of Nano-Glo HiBiT Lytic Buffer, Nano-Glo HiBiT Lytic Substrate and LgBiT protein. This mixture was incubated for 10 minutes at room temperature in the dark. The luminescence was measured using a Tecan M200 microplate reader with an integration time of 1000 ms.

### Quantification of Trastuzumab (Herceptin™, Genentech)

The level of trastuzumab in cell culture supernatant was determined by the anti-HER2 ELISA kit (ab237645, Abcam) following the manufacturer's protocol. This detection method was chosen because a positive signal only occurs in the presence of an intact whole antibody that includes both heavy and light chains.

### Statistical Analysis

The results are expressed as mean±standard deviation (SD). Data were analyzed by Student's t-test, one-way/two-way ANOVA, or Pearson's correlation. The difference between means was considered statistically significant at p<0.05.

### Results

RBCEVs Co-Loaded with a Double-Stranded Decoy Oligonucleotide Increases Transgene Expression in Huh-7, HepG2, THP-1 Cell Lines

RBCEVs were loaded with a DNA plasmid (CMV-copGFP), or simultaneously loaded with a DNA plasmid and NF-κB decoy (ODN), scrambled (SCD), phosphorothioate-modified NF-κB decoy (ODN-PS), or phosphorothioate-modified scrambled (SCD-PS) oligonucleotides at increasing dosages from 12.5 to 100 pmol. The loading efficiency of RBCEVs was measured post-loading. Loaded RBCEVs were transfected to 50,000 Huh-7, HepG2, or THP-1 cells on a 24-well plate format at an equimass amount of the DNA plasmid at 200 ng, 400 ng, and 500 ng respectively. 48 hours after transfection, cells were harvested and analyzed by flow cytometry for GFP expression and cell viability using propidium iodide (PI) staining. Using untreated cells as controls, GFP-positive cells were gated in the FITC channel and Pl-positive cells were gated in the PE channel.

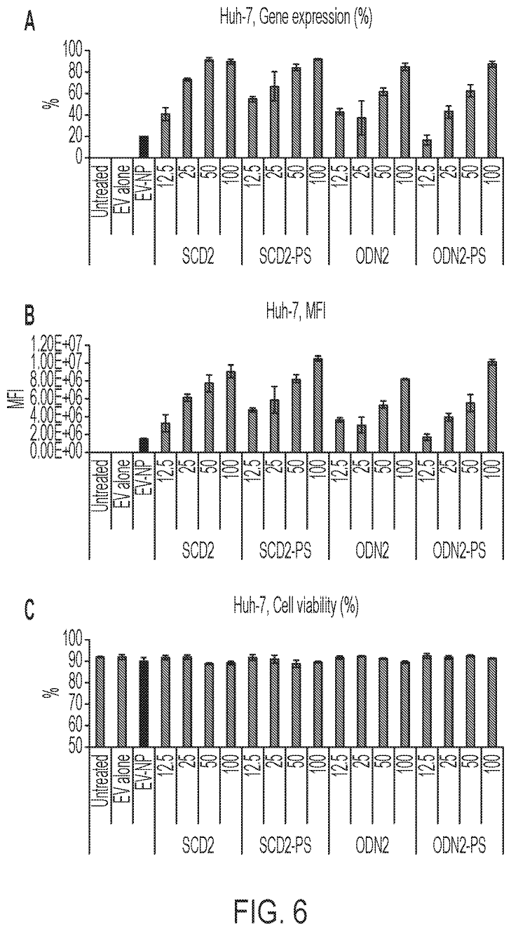

Results obtained are presented in FIG. 6. Increased expression of GFP was observed in cells that were transfected with RBCEVs that were co-loaded with DNA plasmid and ODN or SCD oligonucleotides. Co-loaded RBCEVs increased transgene expression in some cases by roughly a factor of 2, 3, 4, 5, or 6. Cells that were treated with RBCEVs co-loaded with a single DNA plasmid and ODN showed increased transfection efficiency and expression of the DNA payload in cells. The increase in transfection efficiency and transgene expression was observed to be dose-dependent. Specifically, in each case, transgene expression showed greater enhancement after administration of higher oligonucleotide dose. Pre-treatment with oligonucleotides did not negatively impact cell viability.

Administration of RBCEVs Co-Loaded with a Double-Stranded Decoy Oligonucleotide Enhances Transgene Expression in BL/6 and SCID Mice

RBCEVs were loaded with a DNA plasmid that expresses hFIX-HiBit under the LSP promoter (hFIX-HiBit) or co-loaded with DNA plasmid and NF-κB decoy (ODN) or scrambled (SCD) oligonucleotides. RBCEVs were co-loaded with two different dosages of oligonucleotides, at 25 pmol (low dose; ODN-25 and SCD-25) or 100 pmol (high dose; ODN-100 and SCD-100). In this particular experiment, loaded RBCEVs were administered to BL/6 mice via intravenous tail-vein injection at a fixed DNA plasmid dose of 4 mg/kg. Serum was collected on day 1, day 3, and weekly from day 7 after administration, and transgene expression was measured up to day 49 using the Nano-Glo HiBIT Lytic Detection System to quantify HiBiT-tagged protein expression in the animals.

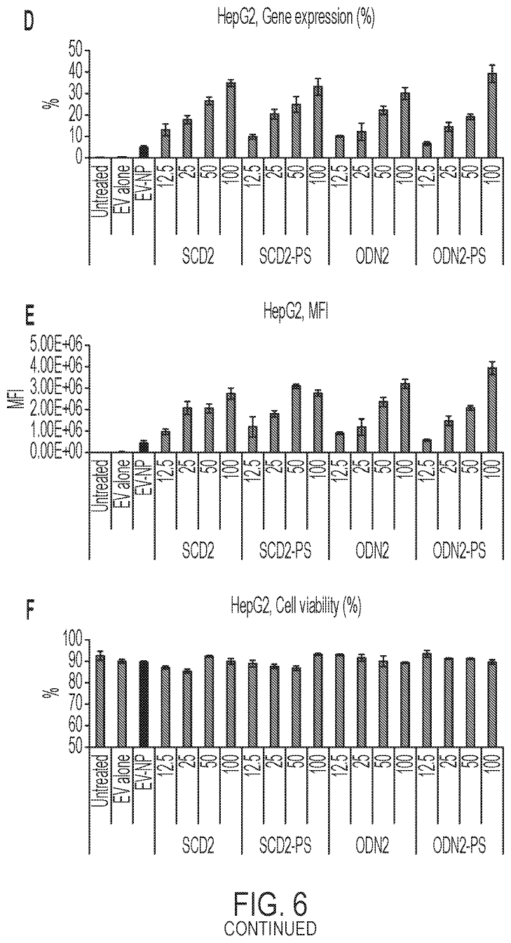

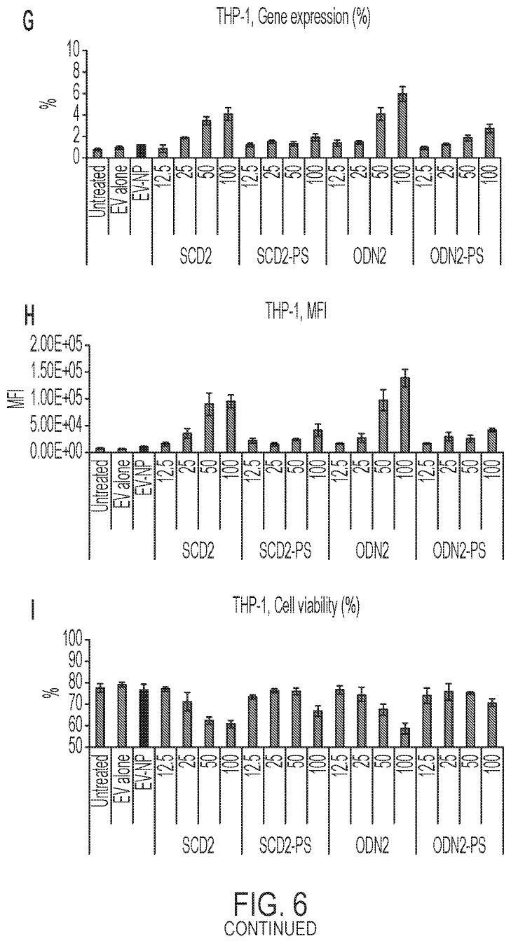

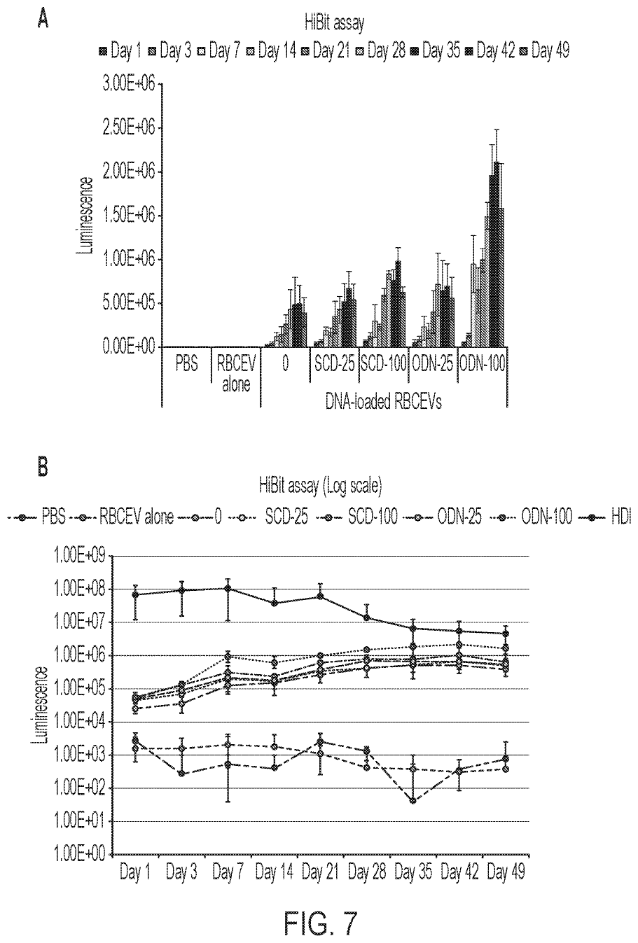

Results obtained are presented in FIG. 7. As can be seen, RBCEVs co-loaded with high dose NF-κB decoy oligonucleotide at 100 pmol (ODN-100) enhanced the HiBit expression as measured by luminescence. High dose NF-κB decoy oligonucleotide treatment resulted in increased HiBit expression at all time-points measured from day 1 up to day 49 as compared to RBCEVs loaded with DNA plasmid alone.

Furthermore, RBCEVs were co-loaded with 3 components: a DNA plasmid that expresses the light chain of monoclonal antibody trastuzumab (HERCEPTIN™, Genentech), a DNA plasmid that expresses the trastuzumab heavy chain, and NF-κB decoy oligonucleotides. In this particular experiment, loaded RBCEVs were administered to SCID mice via intravenous tail-vein injection at a DNA plasmid dose of 4 mg/kg and 6 mg/kg. Serum was collected weekly from day 7, and antibody expression was measured up to day 49 using the anti-HER2 (trastuzumab) ELISA kit (ab237645, Abcam).

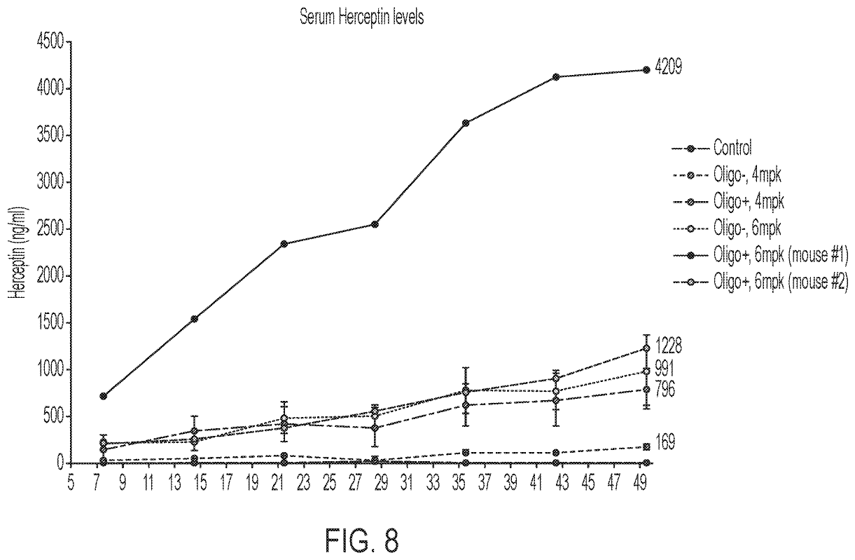

Results obtained are presented in FIG. 8. Expression of trastuzumab was increased in serum of mice that were treated with RBCEVs co-loaded with the trastuzumab light chain and heavy chain plasmids at an equimolar ratio as compared to RBCEVs loaded with the single, bicistronic vector. Co-loaded RBCEVs increased trastuzumab expression in some cases by roughly a factor of 2, 3, 4, 5, or 6.

RBCEVs Co-Loaded with a Double-Stranded Decoy Oligonucleotide Suppresses the Release of type I Interferons (IFN) After IV Injection

In this particular experiment, Type I IFN release was assayed as a measurement of immune response so as to assess the immunological effect of decoy oligonucleotides or scrambled oligonucleotides co-loaded into RBCEVs. The secretion of Type I IFN was quantified after the intravenous tail-vein injection of RBCEVs co-loaded with DNA plasmid and NF-κB decoy (ODN) or scrambled (SCD) oligonucleotides in BL/6 mice. Blood was drawn at 0, 6, and 24 hours post-injection of loaded RBCEVs. Type I IFN induction was quantified from mouse serum using the InvivoGen mouse IFN-beta bioluminescent ELISA kit and mouse IFN-alpha bioluminescent ELISA kit.

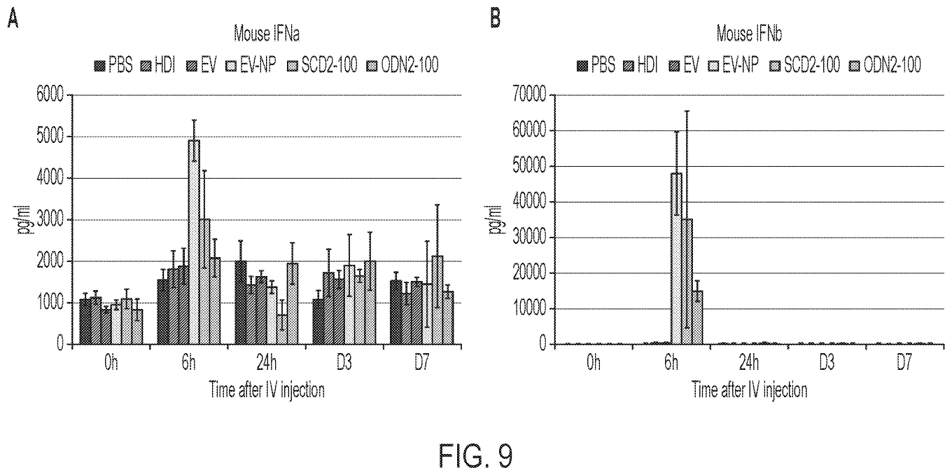

Results obtained are presented in FIG. 9. Administration of RBCEVs loaded with plasmid payload alone induced Type I IFN in vivo. A spike in IFN alpha and beta levels was detected 6 hours post injection in mice that received RBCEVs loaded with DNA plasmid alone (EV-NP). However, animals injected with RBCEVs co-loaded with the addition of NF-κB decoy (ODN) showed significantly reduced levels of IFN alpha and beta levels 6 hours post injection. In some cases, Type I IFN release was suppressed with co-loaded RBCEVs by roughly a factor of 2 or 3. Co-loading of NF-κB decoy oligonucleotides with RBCEVs reduced the induction of Type I IFN in vivo, compared to RBCEVs loaded without NF-κB decoy.

RBCEVs Co-Loaded with Different Double-Stranded Decoy Oligonucleotides Also Increases Transgene Expression in Huh-7 and HepG2 Cell Lines

Different double-stranded oligonucleotides were utilized to assess the effect on transgene expression in vitro with RBCEVs. Table 1 shows the sequences of double-stranded, NF-κB decoy oligonucleotides designed and tested with RBCEVs. RBCEVs were loaded with a DNA plasmid that expresses copGFP under the CMV promoter (CMV-copGFP) or co-loaded with DNA plasmid along with 100 pmol of annealed NF-κB decoy oligonucleotide. Loaded RBCEVs were transfected to 50,000 HepG2 and Huh-7 cells on a 24-well plate format at an equimass amount of the DNA plasmid at 400 ng and 200 ng respectively. 24 and 48 hours after transfection, cells were harvested and analyzed by flow cytometry for GFP expression and cell viability using propidium iodide (PI) staining.

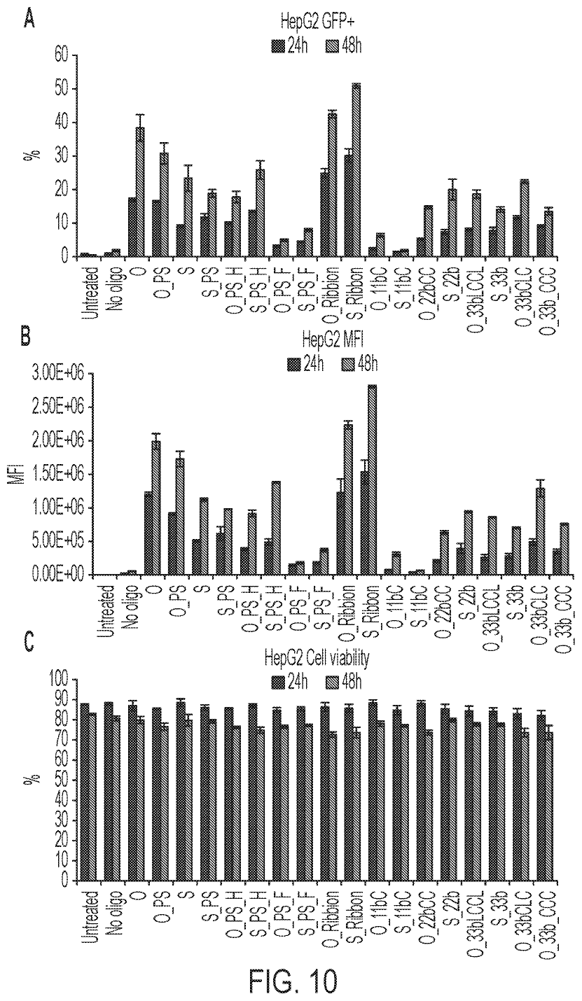

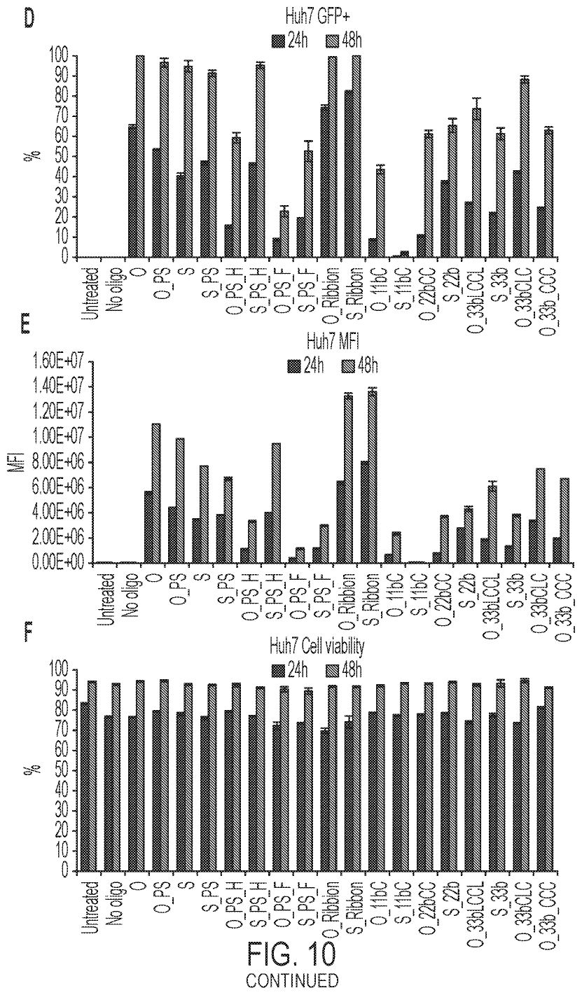

Results obtained are presented in FIG. 10. Cells that were treated with RBCEVs co-loaded with a DNA plasmid and an oligonucleotide design showed increased transfection efficiency and expression of the DNA payload in HepG2 and Huh7 cells as compared to RBCEVs loaded with DNA plasmid alone. All oligonucleotide designs achieved some level of increase of enhancement than its corresponding group lacking an oligonucleotide without reducing cell viability. Co-loaded RBCEVs increased transgene expression in some cases by roughly a factor of 2, 3, 4, 5, or 6. Transgene expression in cells transfected with co-loaded RBCEVs varied with each oligonucleotide design, depending on the composition, length, and structure of the design. For instance, increased phosphorothioate-bonds in the oligonucleotides from end-modifications to full length modifications decreased transfection efficiency. Reducing the length of dsDNA oligonucleotide design from 22 base pairs to 11 base pairs reduced the transfection efficiency of the DNA plasmid. Ribbon shaped oligonucleotide designs outperformed all other designs, which could be due to longer half-life from reduced nuclease degradation of open-ended oligonucleotides.

TABLE 1
Different decoy oligonucleotide designs co-loaded
with RBCEVs. Highlighted in bold is the 11 base
pair NF-KB binding sequence, asterisks represent
phosphorothioate-bond modifications.

| Name | Sequence |
|---|---|
| NFKB_O_For | GATCGAGGGGACTTTCCCTAGC |
| NFKB_O_Rev | GCTAGGGAAAGTCCCCTCGATC |
| NFKB_O_PS_For | G*A*T CGA GGG GAC TTT CCC TA*G *C |
| NFKB_O_PS_Rev | G*C*TAGGGAAAGTCCCCTCGA*T*C |
| NFKB_S_For | ATCTGGCGGTCCAGTTAGAGCC |
| NFKB_S_Rev | GGCTCTAACTGGACCGCCAGAT |
| NFKB_S_PS_For | A*T*CTGGCGGTCCAGTTAGAG*C*C |
| NFKB_S_PS_Rev | G*G*CTCTAACTGGACCGCCAG*A*T |
| NFKB_O_PS_H_For | G*A*T*C*G*AGGGGACTTTCCC *T*A*G*C |
| NFKB_O_PS_H_Rev | G*C*T*A*GGGAAAGTCCCCT*C*G*A*T*C |
| NFKB_S_PS_H_For | A*T*C*T*G*GCGGTCCAGTTAG*A*G*C*C |
| NFKB_S_PS_H_Rev | G*G*C*T*CTAACTGGACCGC*C*A*G*A*T |
| NFKB_O_PS_F_For | G*A*T*C*G*A*G*G*G*G*A*C*T*T*T*C*C*C*T*A*G*C |
| NFKB_O_PS_F_Rev | G*C*T*A*G*G*G*A*A*A*G*T*C*C*C*C*T*C*G*A*T*C |
| NFKB_S_PS_F_For | A*T*C*T*G*G*C*G*G*T*C*C*A*G*T*T*A*G*A*G*C*C |
| NFKB_S_PS_F_Rev | G*G*C*T*C*T*A*A*C*T*G*G*A*C*C*G*C*C*A*G*A*T |
| NFKB_O_Ribbon | /5Phos/CGATCGAAAACGATCGAGGGGACTTTCCCTAGCAAAAGC |
| | TAGGGAAAGTCCCCT |
| NFKB_S_Ribbon | /5Phos/CAGATGAAAACATCTGGCGGTCCAGTTAGAGCCAAAAGG |
| | CTCTAACTGGACCGC |
| NFKB_O_For_11bC | GGGACTTTCCC |
| NFKB_O_Rev_11bC | GGGAAAGTCCC |
| NFKB_S_For_11b | TGGACCCTCTG |
| NFKB_S_Rev_11b | CAGAGGGTCCA |
| NFKB_O_For_22bCC | GGGACTTTCCCGGGACTTTCCC |
| NFKB_O_Rev_22bCC | GGGAAAGTCCCGGGAAAGTCCC |
| NFKB_S_For_22b | TCCCGGCTCGGGCCTTTCATGA |
| NFKB_S_Rev_22b | TCATGAAAGGCCCGAGCCGGGA |
| NFKB_O_For_33bLCCL | GATCGAGGGGACTTTCCCGGGACTTTCCCTAGC |
| NFKB_O_Rev_33bLCCL | GCTAGGGAAAGTCCCGGGAAAGTCCCCTCGATC |
| NFKB_S_For_33b | CAAGGCGCAGATCGTTGCTCAGTTTGTGCCCGC |
| NFKB_S_Rev_33b | GCGGGCACAAACTGAGCAACGATCTGCGCCTTG |
| NFKB_O_For_33bCLC | GGGACTTTCCCTAGCGATCGAGGGGACTTTCCC |
| NFKB_O_Rev_33bCLC | GGGAAAGTCCCCTCGATCGCTAGGGAAAGTCCC |
| NFKB_O_For_33bCCC | GGGACTTTCCCGGGACTTTCCCGGGACTTTCCC |
| NFKB_O_Rev_33bCCC | GGGAAAGTCCCGGGAAAGTCCCGGGAAAGTCCC |

RBCEVs Co-Loaded with Double-Stranded Bait Oligonucleotides Increases Transgene Expression in Huh-7, HepG2, THP-1 Cell Lines

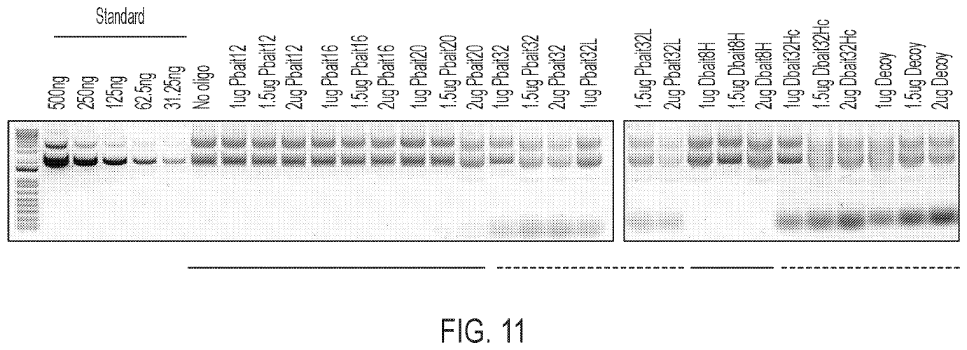

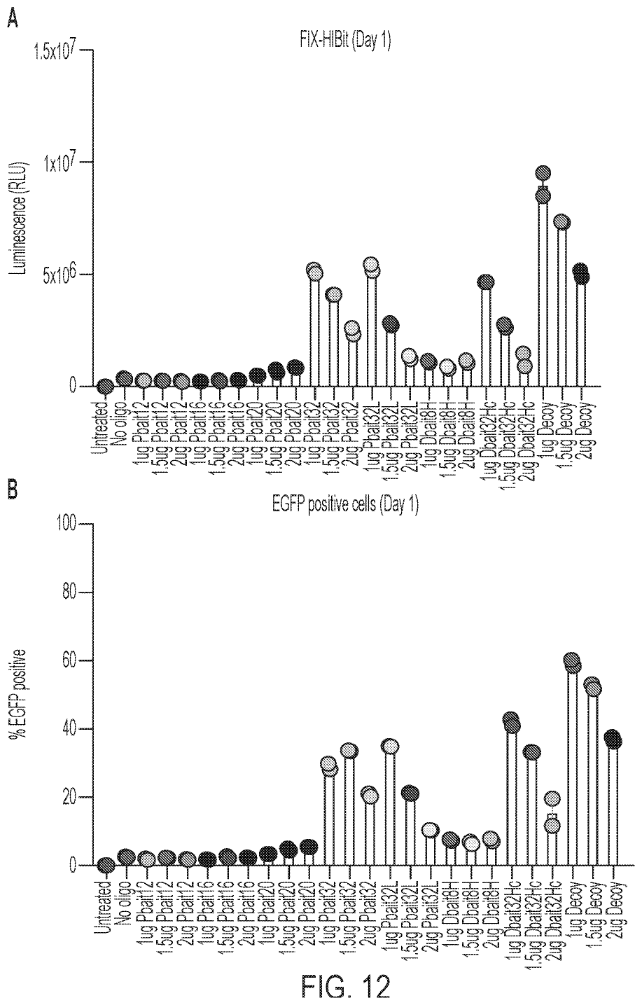

RBCEVs were loaded with two DNA plasmids, one which encodes eGFP and one which encodes FIX-HiBit luciferase reporter construct, or simultaneously loaded with the two plasmids and a self-complementary single-stranded (e.g., comprises one or more double-stranded portions and stem-loop structures) bait oligonucleotide as laid out in Table 2 at increasing dosages from 1 to 2 μg by chemical-based transfection reagent. The bait oligonucleotides in Table 2 are short interfering DNA molecules (siDNA) that mimic either double-strand breaks (Dbait) or single-strand breaks (Pbait). Such oligonucleotides are known to promote DNA-dependent protein kinase (DNA-PK) and/or poly (ADP-ribose) polymerase (PARP) activation (see, for example, Croset, et al., “Inhibition of DNA damage repair by artificial activation of PARP with siDNA.” Nucleic acids research 41.15 (2013)). The loading efficiency of RBCEVs was measured post-loading (FIG. 11). Loaded RBCEVs were transfected to 100,000 Huh-7 cells at an equimass amount of the loaded RBCEVs at 200ng. 24 hours after transfection, supernatant was harvested and Nano-Glo® HiBiT assay was performed to evaluate the transgene expression, and cells were harvested and analyzed by flow cytometry for GFP expression. Results obtained are presented in FIG. 12. As can be seen, several RBCEV compositions loaded with a bait oligonucleotide increased expression of both transgenes. Specifically, Pbait32, Pbait 32L, Dbait8H, and Dbait32Hc increased both eGFP and FIX-HiBit expression at various dosages. These results highlight the effect of double-stranded promoting oligonucleotides to increase the level, expression, and/or activity of a payload nucleic acid, especially when co-loaded into RBCEVs.

TABLE 2
Different bait oligonucleotide designs
co-loaded with RBCEVs. Asterisks represent
phosphorothioate-bond modifications. iSp18
represents hexaethylene glycol spacer.

| Name | Sequence |
|---|---|
| Pbait12 | AAGCACiSp18GTGCTTGGAGCAiSp18 |
| | TGCTCC |
| Pbait16 | AAGCACTGiSp18CAGTGCTTGGAGCAT |
| | GiSp18CATGCTCC |
| Pbait20 | AAGCACTGCAiSp18TGCAGTGCTTGGA |
| | GCATGACiSp18GTCATGCTCC |
| Pbait32 | AACCAGTAGCTCTGGTiSp18ACCAGAG |
| | CTACTGGTTAAGGGCTGACAGGCCA |
| | iSp18TGGCCTGTCAGCCCTT |
| Pbait32L | AAGCACTGCACACAGCiSp18GCTGTGT |
| | GCAGTGCTTGGAGCATG |
| | ACCCTAGAiSp18TCTAGGGTCATGCTCC |
| Dbait8H | G*C*T*AGGTCiSp18GACCT*A*G*C |
| Dbait32Hc | G*C*T*GTGCCCACAACCCAGCAAACAAG |
| | CCTAGAiSp18TCTAGG |
| | CTTGTTTGCTGGGTTGTGGGCAC*A*G*C |

### Example 3

The present Example demonstrates that co-loading of RBCEVs with single-stranded oligonucleotides can increase transgene expression when delivered in vitro while also suppressing the immune response to the transgene. Specifically, transgene expression and inflammatory cytokine expression were assessed after co-loaded RBCEVs were delivered to multiple human cell lines of different tissue origin.

RBCEVs were loaded with a DNA plasmid that expresses hFIX-HiBit luciferase reporter construct or co-loaded with a DNA plasmid that expresses hFIX-HiBit luciferase reporter construct and a double-stranded oligonucleotide (scrambled, NF-κB decoy) or a single-stranded oligonucleotide. Table 3 shows the sequences of single-stranded, NF-κB decoy oligonucleotides designed and tested with RBCEVs. 100,000 of Huh7 and HepG2 cells were transfected with the loaded RBCEVs containing 200 ng of DNA plasmid. Cell culture media was collected on day 1 post transfection and Nano-Glo® HiBiT assay was performed to evaluate the transgene expression.

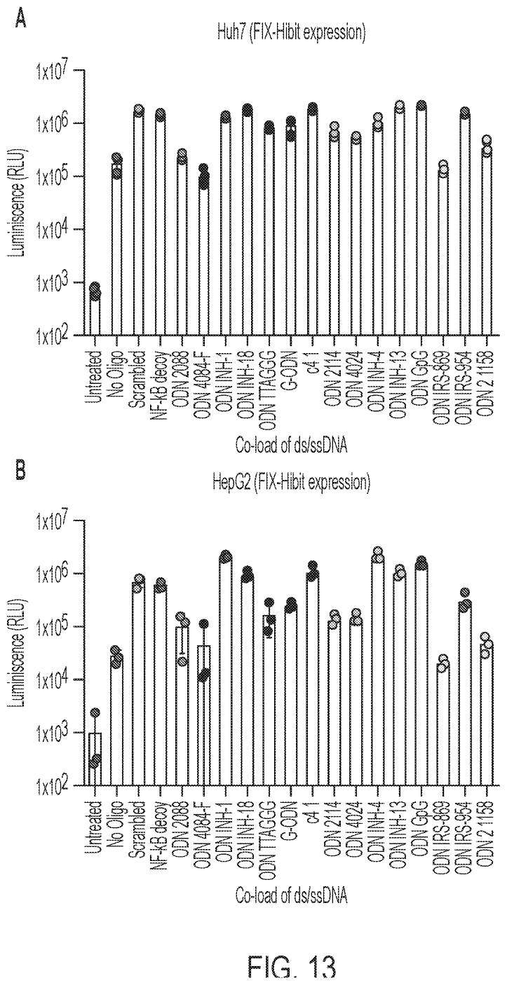

Results obtained are presented in FIG. 13. Multiple different designs of single-stranded oligonucleotide increased transgene expression as compared to RBCEVs loaded without oligonucleotide. Specifically, RBCEVs loaded with a single-stranded oligonucleotide increased FIX-Hibit expression and/or activity in multiple cell lines when co-loaded with FIX-HiBit construct.

Furthermore, RBCEVs were loaded with a DNA plasmid that expresses hFIX-HiBit luciferase reporter construct or co-loaded with a DNA plasmid that expresses hFIX-HiBit luciferase reporter and double-stranded oligonucleotide (scrambled, NF-κB decoy) or single-stranded oligonucleotide. Table 3 shows the sequences of single-stranded, NF-κB decoy oligonucleotides designed and tested with RBCEVs. 500,000 of THP-1 cell line was transfected with the loaded RBCEVs containing 2000 ng of DNA plasmid and cells were collected at 6 hours post transfection for gene expression analysis by quantitative PCR (qPCR).

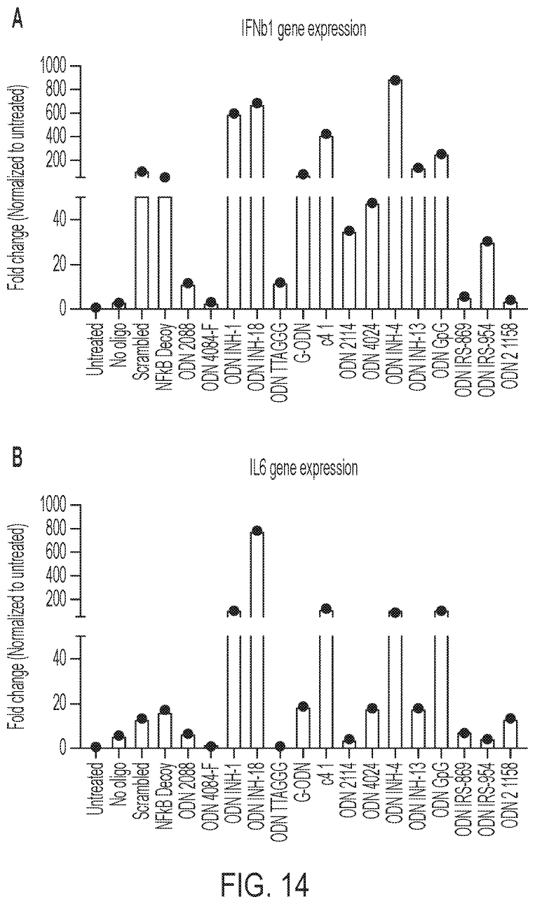

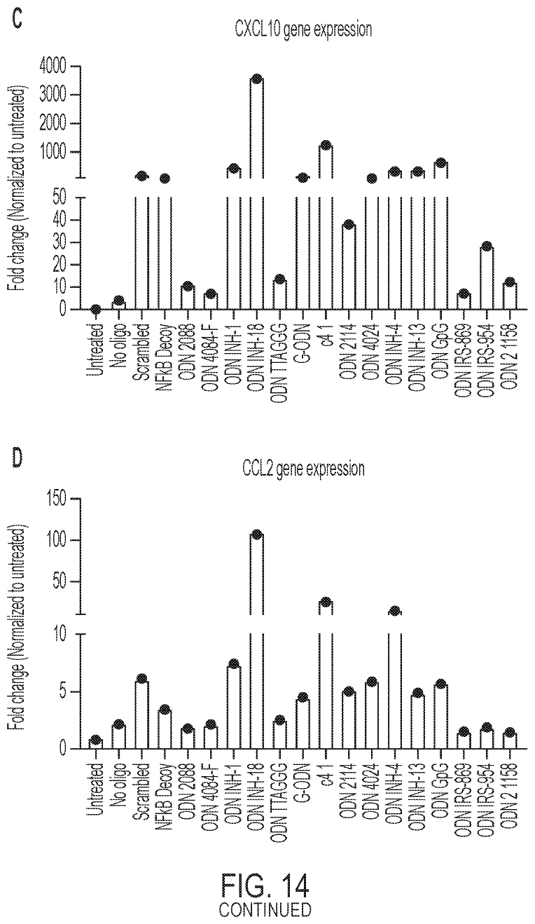

Results obtained are presented in FIG. 14. As can be seen, RBCEVs co-loaded with certain single-stranded oligonucleotides can suppress cytokine expression when delivered to cells. Specifically, multiple different designs of single-stranded oligonucleotide decreased expression of IFNb1, IL6, CXCL10, and/or CCL2 gene expression. These results showcase the effect of single-stranded promoting oligonucleotides to decrease inflammatory and/or otherwise undesirable effect or response (e.g., cytokine expression) associated with delivery of a payload nucleic acid.

TABLE 3
Single stranded oligodeoxynucleotide
designs co-loaded with RBCEVs.

| Name | Sequence |
|---|---|
| ODN 2088 | TCCTGGCGGGGAAGT |
| ODN 4084-F | CCTGGATGGGAA |
| ODN INH-1 | CCTGGATGGGAATTCCCATCCAGG |
| ODN INH-18 | CCTGGATGGGAACTTACCGCTGCA |
| ODN TTAGGG | TTAGGGTTAGGGTTAGGGTTAGGG |
| G-ODN | CTCCTATTGGGGGTTTCCTAT |
| c4 1 | TGGCGCGCACCCACGGCCTG |
| ODN 2114 | TCCTGGAGGGGAAGT |
| ODN 4024 | TCCTGGATGGGAAGT |
| ODN INH-4 | TTCCCATCCAGGCCTGGATGGGAA |
| ODN INH-13 | CTTACCGCTGCACCTGGATGGGAA |
| ODN GpG | TGACTGTGAAGGTTAGAGATGA |
| ODN IRS-869 | TCCTGGAGGGGTTGT |
| ODN IRS-954 | TGCTCCTGGAGGGGTTGT |
| ODN 2 1158 | CCTGGCGGGG |

1. A population of extracellular vesicles loaded with a cargo, wherein the cargo comprises at least two different nucleic acid molecules and the population is prepared by contacting the extracellular vesicles with the cargo in the presence of a transfection reagent.

2. The population of claim 1, wherein the transfection reagent is a cationic lipid reagent.

3. The population of claim 2, wherein the transfection reagent is selected from the group consisting of Lipofectamine™ 3000™ (ThermoFisher), Turbofect™ (ThermoFisher), Lipofectamine™ MessengerMAX™ (ThermoFisher), Exofect™ (System Biosciences), and Linear Polyethylenimine Hydrochlorides.

4. The population of claim 3, wherein the transfection reagent is a Linear Polyethylenimine Hydrochloride having an average molecular weight of 25,000 Da or 40,000 Da.

5. The population of claim 4, wherein the transfection reagent is PEIMax™ (Polysciences, Inc.) or jetPEI® (Polyplus transfection).

6. An extracellular vesicle loaded with a cargo, wherein the cargo comprises at least two different nucleic acid molecules.

7. The extracellular vesicle of claim 6, wherein the at least two different nucleic acid molecules have non-identical sequences.

8. The extracellular vesicle of claim 6 or claim 7, wherein the at least two different nucleic acid molecules are different types of nucleic acid molecule.

9. The extracellular vesicle of any one of claims 6-8 wherein the nucleic acid molecule is selected from the group consisting of: a DNA plasmid, an RNA plasmid, a DNA minicircle, a dumbbell-shaped DNA minimal vector, an RNA minicircle, a circular DNA, a linear double-stranded DNA, a small interfering RNA (siRNA), a messenger RNA (mRNA), a guide RNA (gRNA), a prime editing guide RNA (peg RNA), a CRISPR RNA (crRNA), a trans-activating CRISPR RNA (tracrRNA), a circular RNA, a microRNA (miRNA), a primary miRNA (pri-miRNA), a precursor miRNA (pre-miRNA), a piwi-interacting RNA (piRNA), a transfer RNA (tRNA), a long noncoding RNA (IncRNA), an antisense oligonucleotide (ASO), a short hairpin RNA (shRNA), a small activating RNA (saRNA), a small nucleolar RNAs (snoRNA), a gapmer, a locked nucleic acid (LNA), a peptide nucleic acid (PNA), and an expression vector.

10. The extracellular vesicle of any one of the preceding claims wherein the cargo comprises: a DNA and an RNA, at least two different mRNA molecules, a plasmid and a gRNA, an mRNA and a gRNA, a plasmid and an antisense oligonucleotide, a plasmid and a siRNA.

11. The extracellular vesicle of claim 6 wherein the cargo comprises components of a CRISPR/Cas gene editing system.

12. The extracellular vesicle of claim 11 wherein the cargo comprises a gRNA molecule and a nucleic acid molecule encoding a nuclease, wherein preferably the nuclease is a Cas9 nuclease or a Cas12 nuclease.

13. The extracellular vesicle of claim 12, wherein the gRNA is an sgRNA or pegRNA.

14. The extracellular vesicle of claim 12 or claim 13, wherein the cargo further comprises a DNA repair template.

15. An extracellular vesicle comprising an mRNA or plasmid encoding an antigen-binding molecule or fragment thereof.

16. The extracellular vesicle of claim 15 wherein the extracellular vesicle comprises a first nucleic acid encoding a first polypeptide of the antigen-binding molecule, and a second nucleic acid encoding a second polypeptide of the antigen-binding molecule.

17. The extracellular vesicle of claim 15 or claim 16 wherein the antigen-binding molecule is an antibody or an antigen-binding fragment thereof.

18. The extracellular vesicle of claim 17, wherein the antigen-binding molecule is an antibody or an scFv.

19. The extracellular vesicle of any one of the preceding claims wherein the extracellular vesicle is derived from a red blood cell.

20. A composition comprising extracellular vesicles, wherein at least one of the extracellular vesicles in the composition is an extracellular vesicle according to any one of claims 6 to 19.

21. A method for loading an extracellular vesicle with a cargo, the method comprising:
a. providing a mixture, the mixture comprising cargo molecules to be loaded into an extracellular vesicle; and
b. contacting with an extracellular vesicle under conditions sufficient for the extracellular vesicle to be loaded with the cargo molecules;
wherein the mixture of cargo molecules comprises at least two different nucleic acid molecules.

22. The method of claim 21 wherein the mixture further comprises a transfection reagent.

23. The method of claim 21 or claim 22 wherein the mixture comprises the at least two different nucleic acid molecules in a ratio of about 1:1 or between 2:1-1:2 or between 3:1-1:3.

24. The method of any one of claims 21-23 wherein the mixture is prepared by combining the at least two nucleic acid molecules to provide a mixture of nucleic acid molecules, and subsequently contacting the mixture of cargo molecules with the transfection reagent.

25. The method of claim any one of claims 21 to 24, wherein the at least two different nucleic acid molecules have non-identical sequences.

26. The method of any one of claims 21 to 25 wherein the at least two different nucleic acid molecules are different types of nucleic acid molecule.

27. The method of any one of claims 21 to 26 wherein the nucleic acid molecule is selected from the group consisting of: a DNA plasmid, a small interfering RNA (siRNA), a messenger RNA (mRNA), a guide RNA (gRNA), a prime editing guide RNA (peg RNA), a CRISPR RNA (crRNA), a trans-activating CRISPR RNA (tracrRNA), a circular RNA, a microRNA (miRNA), a primary miRNA (pri-miRNA), a precursor miRNA (pre-miRNA), a piwi-interacting RNA (piRNA), a transfer RNA (tRNA), a long noncoding RNA (IncRNA), an antisense oligonucleotide (ASO), a short hairpin RNA (shRNA), a small activating RNA (saRNA), a small nucleolar RNAS (snoRNA), a gapmer, a locked nucleic acid (LNA), a peptide nucleic acid (PNA), an expression vector, a circular DNA, a linear double stranded DNA, an RNA plasmid, a DNA minicircle, a dumbbell-shaped DNA minimal vector and an RNA minicircle.

28. The method of any one of claims 21 to 27 wherein the cargo comprises: a DNA and an RNA, at least two different mRNA molecules, a plasmid and a gRNA, or an mRNA and a gRNA, a plasmid and an antisense oligonucleotide, a plasmid and a siRNA.

29. The method of any one of claims 21 to 28 wherein the cargo comprises components of a CRISPR/Cas gene editing system.

30. The method of claim 29 wherein the cargo comprises a gRNA molecule and a nucleic acid molecule encoding a nuclease, wherein preferably the nuclease is a Cas9 nuclease or a Cas12 nuclease.

31. The method according to any one of claims 21 to 27 wherein the cargo comprises two or more nucleic acid molecules that encode an antigen binding molecule.

32. The method of claim 31 wherein the extracellular vesicle comprises a first nucleic acid encoding a first polypeptide of the antigen-binding molecule, and a second nucleic acid encoding a second polypeptide of the antigen-binding molecule.

33. The method of any one of claims 21 to 32 wherein the extracellular vesicle is derived from a red blood cell.

34. A method for delivering two or more nucleic acids to a cell, the method comprising contacting the cell with one or more extracellular vesicles according to any one of claims 6 to 20.

35. An extracellular vesicle according to any one of claims 6 to 20 for use in a method of treatment.

36. A method of treatment, the method comprising administering an extracellular vesicle according to claim 6 to a patient in need of treatment.

37. Use of an extracellular vesicle according to any one of claims 6 to 20 in the manufacture of a medicament for the treatment of a disease or a disorder.

38. The extracellular vesicle for use, method of treatment, or use according to any one of claims 35-37 wherein the method of treatment involves administration of a extracellular vesicle according to any one of claims 6 to 20 to a subject with a genetic disorder, inflammatory disease, cancer, autoimmune disease, cardiovascular disease or a gastrointestinal disease.

39. The extracellular vesicle for use, method of treatment, or use according to claim 38 wherein the subject has cancer, the cancer optionally selected from leukemia, lymphoma, myeloma, breast cancer, lung cancer, liver cancer, colorectal cancer, nasopharyngeal cancer, kidney cancer or glioma.

40. The extracellular vesicle for use, method of treatment, or use according to any one of claims 37 to 39 wherein the method involves treatment of a disease in the patient by expression of a protein or peptide encoded by the nucleic acid cargo.


SEQUENCE LISTING (66 sequences)

SEQ ID NO: 1 - protein, 5 aa, Artificial Sequence
  Description of Artificial Sequence Synthetic peptide
  MOD_RES (3)..(3): Any naturally occurring amino acid
LPXTG

SEQ ID NO: 2 - protein, 5 aa, Artificial Sequence
  Description of Artificial Sequence Synthetic peptide
LPETG

SEQ ID NO: 3 - protein, 6 aa, Artificial Sequence
  Description of Artificial Sequence Synthetic 6xHis tag
HHHHHH

SEQ ID NO: 4 - DNA, 12 bp, Artificial Sequence
  Description of Artificial Sequence Synthetic oligonucleotide
ggggacttttcc

SEQ ID NO: 5 - DNA, 22 bp, Artificial Sequence
  Description of Artificial Sequence Synthetic oligonucleotide
gatcgaggggactttccctagc

SEQ ID NO: 6 - DNA, 22 bp, Artificial Sequence
  Description of Artificial Sequence Synthetic oligonucleotide
gctagggaaagtcccctcgatc

SEQ ID NO: 7 - DNA, 22 bp, Artificial Sequence
  Description of Artificial Sequence Synthetic oligonucleotide
atctggcggtccagttagagcc

SEQ ID NO: 8 - DNA, 22 bp, Artificial Sequence
  Description of Artificial Sequence Synthetic oligonucleotide
ggctctaactggaccgccagat

SEQ ID NO: 9 - DNA, 22 bp, Artificial Sequence
  Description of Artificial Sequence Synthetic oligonucleotide
gatcgaggggactttccctagc

SEQ ID NO: 10 - DNA, 22 bp, Artificial Sequence
  Description of Artificial Sequence Synthetic oligonucleotide
gctagggaaagtcccctcgatc

SEQ ID NO: 11 - DNA, 22 bp, Artificial Sequence
  Description of Artificial Sequence Synthetic oligonucleotide
atctggcggtccagttagagcc

SEQ ID NO: 12 - DNA, 22 bp, Artificial Sequence
  Description of Artificial Sequence Synthetic oligonucleotide
ggctctaactggaccgccagat

SEQ ID NO: 13 - DNA, 22 bp, Artificial Sequence
  Description of Artificial Sequence Synthetic oligonucleotide
gatcgaggggactttccctagc

SEQ ID NO: 14 - DNA, 22 bp, Artificial Sequence
  Description of Artificial Sequence Synthetic oligonucleotide
gctagggaaagtcccctcgatc

SEQ ID NO: 15 - DNA, 22 bp, Artificial Sequence
  Description of Artificial Sequence Synthetic oligonucleotide
atctggcggtccagttagagcc

SEQ ID NO: 16 - DNA, 22 bp, Artificial Sequence
  Description of Artificial Sequence Synthetic oligonucleotide
ggctctaactggaccgccagat

SEQ ID NO: 17 - DNA, 22 bp, Artificial Sequence
  Description of Artificial Sequence Synthetic oligonucleotide
gatcgaggggactttccctagc

SEQ ID NO: 18 - DNA, 22 bp, Artificial Sequence
  Description of Artificial Sequence Synthetic oligonucleotide
gctagggaaagtcccctcgatc

SEQ ID NO: 19 - DNA, 22 bp, Artificial Sequence
  Description of Artificial Sequence Synthetic oligonucleotide
atctggcggtccagttagagcc

SEQ ID NO: 20 - DNA, 22 bp, Artificial Sequence
  Description of Artificial Sequence Synthetic oligonucleotide
ggctctaactggaccgccagat

SEQ ID NO: 21 - DNA, 54 bp, Artificial Sequence
  Description of Artificial Sequence Synthetic oligonucleotide
cgatcgaaaacgatcgaggggactttccctagcaaaagctagggaaagtcccct

SEQ ID NO: 22 - DNA, 54 bp, Artificial Sequence
  Description of Artificial Sequence Synthetic oligonucleotide
cagatgaaaacatctggcggtccagttagagccaaaaggctctaactggaccgc

SEQ ID NO: 23 - DNA, 11 bp, Artificial Sequence
  Description of Artificial Sequence Synthetic oligonucleotide
gggactttccc

SEQ ID NO: 24 - DNA, 11 bp, Artificial Sequence
  Description of Artificial Sequence Synthetic oligonucleotide
gggaaagtccc

SEQ ID NO: 25 - DNA, 11 bp, Artificial Sequence
  Description of Artificial Sequence Synthetic oligonucleotide
tggaccctctg

SEQ ID NO: 26 - DNA, 11 bp, Artificial Sequence
  Description of Artificial Sequence Synthetic oligonucleotide
cagagggtcca

SEQ ID NO: 27 - DNA, 22 bp, Artificial Sequence
  Description of Artificial Sequence Synthetic oligonucleotide
gggactttcccgggactttccc

SEQ ID NO: 28 - DNA, 22 bp, Artificial Sequence
  Description of Artificial Sequence Synthetic oligonucleotide
gggaaagtcccgggaaagtccc

SEQ ID NO: 29 - DNA, 22 bp, Artificial Sequence
  Description of Artificial Sequence Synthetic oligonucleotide
tcccggctcgggcctttcatga

SEQ ID NO: 30 - DNA, 22 bp, Artificial Sequence
  Description of Artificial Sequence Synthetic oligonucleotide
tcatgaaaggcccgagccggga

SEQ ID NO: 31 - DNA, 33 bp, Artificial Sequence
  Description of Artificial Sequence Synthetic oligonucleotide
gatcgaggggactttcccgggactttccctagc

SEQ ID NO: 32 - DNA, 33 bp, Artificial Sequence
  Description of Artificial Sequence Synthetic oligonucleotide
gctagggaaagtcccgggaaagtcccctcgatc

SEQ ID NO: 33 - DNA, 33 bp, Artificial Sequence
  Description of Artificial Sequence Synthetic oligonucleotide
caaggcgcagatcgttgctcagtttgtgcccgc

SEQ ID NO: 34 - DNA, 33 bp, Artificial Sequence
  Description of Artificial Sequence Synthetic oligonucleotide
gcgggcacaaactgagcaacgatctgcgccttg

SEQ ID NO: 35 - DNA, 33 bp, Artificial Sequence
  Description of Artificial Sequence Synthetic oligonucleotide
gggactttccctagcgatcgaggggactttccc

SEQ ID NO: 36 - DNA, 33 bp, Artificial Sequence
  Description of Artificial Sequence Synthetic oligonucleotide
gggaaagtcccctcgatcgctagggaaagtccc

SEQ ID NO: 37 - DNA, 33 bp, Artificial Sequence
  Description of Artificial Sequence Synthetic oligonucleotide
gggactttcccgggactttcccgggactttccc

SEQ ID NO: 38 - DNA, 33 bp, Artificial Sequence
  Description of Artificial Sequence Synthetic oligonucleotide
gggaaagtcccgggaaagtcccgggaaagtccc

SEQ ID NO: 39 - DNA, 12 bp, Artificial Sequence
  Description of Artificial Sequence Synthetic oligonucleotide
gtgcttggagca

SEQ ID NO: 40 - DNA, 16 bp, Artificial Sequence
  Description of Artificial Sequence Synthetic oligonucleotide
cagtgcttggagcatg

SEQ ID NO: 41 - DNA, 10 bp, Artificial Sequence
  Description of Artificial Sequence Synthetic oligonucleotide
aagcactgca

SEQ ID NO: 42 - DNA, 20 bp, Artificial Sequence
  Description of Artificial Sequence Synthetic oligonucleotide
tgcagtgcttggagcatgac

SEQ ID NO: 43 - DNA, 10 bp, Artificial Sequence
  Description of Artificial Sequence Synthetic oligonucleotide
gtcatgctcc

SEQ ID NO: 44 - DNA, 16 bp, Artificial Sequence
  Description of Artificial Sequence Synthetic oligonucleotide
aaccagtagctctggt

SEQ ID NO: 45 - DNA, 32 bp, Artificial Sequence
  Description of Artificial Sequence Synthetic oligonucleotide
accagagctactggttaagggctgacaggcca

SEQ ID NO: 46 - DNA, 16 bp, Artificial Sequence
  Description of Artificial Sequence Synthetic oligonucleotide
tggcctgtcagccctt

SEQ ID NO: 47 - DNA, 16 bp, Artificial Sequence
  Description of Artificial Sequence Synthetic oligonucleotide
aagcactgcacacagc

SEQ ID NO: 48 - DNA, 32 bp, Artificial Sequence
  Description of Artificial Sequence Synthetic oligonucleotide
gctgtgtgcagtgcttggagcatgaccctaga

SEQ ID NO: 49 - DNA, 16 bp, Artificial Sequence
  Description of Artificial Sequence Synthetic oligonucleotide
tctagggtcatgctcc

SEQ ID NO: 50 - DNA, 32 bp, Artificial Sequence
  Description of Artificial Sequence Synthetic oligonucleotide
gctgtgcccacaacccagcaaacaagcctaga

SEQ ID NO: 51 - DNA, 32 bp, Artificial Sequence
  Description of Artificial Sequence Synthetic oligonucleotide
tctaggcttgtttgctgggttgtgggcacagc

SEQ ID NO: 52 - DNA, 15 bp, Artificial Sequence
  Description of Artificial Sequence Synthetic oligonucleotide
tcctggcggggaagt

SEQ ID NO: 53 - DNA, 12 bp, Artificial Sequence
  Description of Artificial Sequence Synthetic oligonucleotide
cctggatgggaa

SEQ ID NO: 54 - DNA, 24 bp, Artificial Sequence
  Description of Artificial Sequence Synthetic oligonucleotide
cctggatgggaattcccatccagg

SEQ ID NO: 55 - DNA, 24 bp, Artificial Sequence
  Description of Artificial Sequence Synthetic oligonucleotide
cctggatgggaacttaccgctgca

SEQ ID NO: 56 - DNA, 24 bp, Artificial Sequence
  Description of Artificial Sequence Synthetic oligonucleotide
ttagggttagggttagggttaggg

SEQ ID NO: 57 - DNA, 21 bp, Artificial Sequence
  Description of Artificial Sequence Synthetic oligonucleotide
ctcctattgggggtttcctat

SEQ ID NO: 58 - DNA, 20 bp, Artificial Sequence
  Description of Artificial Sequence Synthetic oligonucleotide
tggcgcgcacccacggcctg

SEQ ID NO: 59 - DNA, 15 bp, Artificial Sequence
  Description of Artificial Sequence Synthetic oligonucleotide
tcctggaggggaagt

SEQ ID NO: 60 - DNA, 15 bp, Artificial Sequence
  Description of Artificial Sequence Synthetic oligonucleotide
tcctggatgggaagt

SEQ ID NO: 61 - DNA, 24 bp, Artificial Sequence
  Description of Artificial Sequence Synthetic oligonucleotide
ttcccatccaggcctggatgggaa

SEQ ID NO: 62 - DNA, 24 bp, Artificial Sequence
  Description of Artificial Sequence Synthetic oligonucleotide
cttaccgctgcacctggatgggaa

SEQ ID NO: 63 - DNA, 22 bp, Artificial Sequence
  Description of Artificial Sequence Synthetic oligonucleotide
tgactgtgaaggttagagatga

SEQ ID NO: 64 - DNA, 15 bp, Artificial Sequence
  Description of Artificial Sequence Synthetic oligonucleotide
tcctggaggggttgt

SEQ ID NO: 65 - DNA, 18 bp, Artificial Sequence
  Description of Artificial Sequence Synthetic oligonucleotide
tgctcctggaggggttgt

SEQ ID NO: 66 - DNA, 10 bp, Artificial Sequence
  Description of Artificial Sequence Synthetic oligonucleotide
cctggcgggg